(19) United States
(12) Patent Application Publication
(10) Pub. No.: US 2010/0087002 A1
(43) Pub. Date: Apr. 8, 2010

(54) Methods, Surface Modified Plates and Compositions for Cell Attachment, Cultivation and Detachment

(76) Inventor: Benjamin Fryer, Skillman, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/608,584

(22) Filed: Oct. 29, 2009

Related U.S. Application Data
(63) Continuation-in-part of application No. 12/388,930, filed on Feb. 19, 2009.
(60) Provisional application No. 61/030,544, filed on Feb. 21, 2008.

(51) Int. Cl. C12N 5/00

(52) U.S. Cl. 435/378; 435/395

(57) ABSTRACT

The present invention relates to the field of mammalian cell culture, and provides methods and compositions for cell attachment to, cultivation on and detachment from a solid substrate surface containing from at least about 0.5% N, a sum of O and N of greater than or equal to 17.2% and a contact angle of at least about 13.9 degrees, lacking a feeder cell layer and lacking an adlayer. In one embodiment of the present invention, the cells are treated with a compound capable of inhibiting Rho kinase activity. In another embodiment, the cells are treated with a compound capable of inhibiting Rho activity.

This application is a continuation in part of application Ser. No. 12/388,930, filed Feb. 19, 2009, which claims priority of U.S. provisional application No. 61/030,544 filed Feb. 21, 2008.

### FIELD OF THE INVENTION

The present invention relates to the field of mammalian cell culture, and provides methods and compositions for cell attachment to, cultivation on, and detachment from a solid substrate surface containing from at least about 0.5% N, a sum of O and N of greater than or equal to 17.2% and a contact angle of at least about 13.9 degrees, lacking a feeder cell layer and lacking an adlayer. In one embodiment of the present invention, the cells are treated with a compound capable of inhibiting Rho kinase activity. In another embodiment, the cells are treated with a compound capable of inhibiting Rho activity.

### BACKGROUND

Cultivation of mammalian cells is one of many processes in the life and health sciences. Vessels for mammalian cell culture and analysis involving anchorage-dependent cells are often made of glass or a polymer, such as, for example, polystyrene, that frequently requires additional surface treatment to allow the cells to attach to the surface of the vessel. Such treatments may include applying an adlayer on the surface, for example, by adsorption, grafting or plasma polymerization techniques. Alternatively, the surface treatment may be via chemical modification of the vessel surface itself, which can be achieved by, for example, atmospheric corona, radio frequency vacuum plasma, DC glow discharge, and microwave plasma treatments. These surface treatments change the composition of elements and chemical groups in the surface. The particular chemistry that results depends on the surface treatment method, energy, and time, as well as the composition of the gasses used.

For example, U.S. Pat. No. 5,449,383 discloses a substrate comprising a bulk polymeric material; and a thin polymeric layer which is suitable for supporting cell growth, comprising a reorientation resistant polymer comprising plasma-polymerized amide monomers presenting amide groups for the attachment of cells, wherein said amide monomers are selected from the group of dimethyl formamide and amides having the formula R¹—CO—N(R²)R³ wherein R¹ is an aliphatic, alicyclic, or aromatic group, each of which may be optionally substituted by halogen atoms or hydroxyl groups, and R² and R³ are each independently hydrogen or an alkyl group, and wherein said thin polymer layer promotes attachment and proliferation of said cells.

In another example, EP0348969A1 discloses a method for endothelialization of a polymeric surface comprising contacting a polymeric surface with a plasma generated from a gaseous material comprising nitrogen whereby said polymeric surface is modified to contain surface amino groups, and applying to said modified surface sufficient endothelial cells to form a confluent layer of cells on said amino group-containing surface without a requirement for cell proliferation.

In another example, EP0092302A2 discloses a method for influencing the growth of cell culture in a growth media on a substrate, characterized in that the surface chemistry of the substrate is modified by subjecting the surface of the substrate to a plasma, which is produced from carbon, hydrogen, oxygen, nitrogen, sulphur, phosphorus, a halogen, or a compound of any one of these elements.

In another example, U.S. Pat. No. 6,617,152 B2 discloses an apparatus for treating a polymeric substrate surface comprising: (a) a gas inlet, a microwave energy source and a plasma mixing chamber, the plasma mixing chamber in fluid communication with both the gas inlet and the microwave energy source; (b) a dual chambered treatment area having an inner treatment chamber contained within an outer treatment chamber, said inner treatment chamber having an opening in fluid communication with said outer chamber; (c) said plasma mixing chamber in fluid communication with said outer treatment chamber by means of an aperture; (d) a vacuum outlet line attached to said outer chamber; and (e) whereby said opening in said inner treatment chamber is aligned with said aperture, said opening being spaced from said aperture at predetermined distance.

In one example, US2003/0180903A1 discloses a polymeric substrate having a working surface upon which cells can be cultured wherein the surface oxygen content is at least 25 percent as measured by electron microscopy for chemical analysis at depth about 50 Angstroms.

In one example, WO2006114098 discloses a micro-structured biocompatible material for surgical implants and cell guiding tissue culture surfaces. The microstructure of the biomaterial surface is selected to promote growth of undifferentiated ES cells; promote neuronal differentiation of ES cells; or promote differentiation of ES cells.

In another example, Bigdeli et al. (J. Biotechnol. 133:146-153, 2008) describes a method of adaptation and/or selection of human embryonic stem cells to be cultivated without differentiation under feeder-cell free conditions and without prior treatment of the solid substrate surface with extracellular matrix protein, involving (i) changing media from medium conditioned by human diploid embryonic lung fibroblasts to medium conditioned by neonatal chondrocytes; (ii) then passaging the cells enzymatically from the mouse embryonic feeder cell layer to MATRIGEL®-treated plates, then to COSTAR™ plates, and, finally, to PRIMARIA™ plates; and (iii) changing back to the first used medium again. Very few of the human embryonic stem cells subjected to this method gave rise to established cell lines, suggesting that this method involves selection of human embryonic stem cells to the culture conditions.

Surface treatments that change the composition of elements and chemical groups in the surface itself have successfully been used for preparing polymer solid substrates for the culture of many types of mammalian cells. However, there are significant limitations in terms of poor attachment and/or cultivation using certain types of mammalian cells, for example, pluripotent stem cells and human embryonic kidney (HEK) 293 cells.

Graham et al., (J. Gen. Virol. 36:59-72, 1977) disclose the generation of the cell line HEK293.

HEK293 cell attachment may be enhanced by making an adlayer on the solid substrate surface, using, for example, extracellular matrix proteins, polylysine, polyornithine, or polyethyleneimine, before adding the HEK293 cells to the culture vessel. Preparing the adlayer is, however, time-consuming, and typically results in a non-sterile solid substrate with a shorter shelf life than the bare solid substrate. Therefore, there is a significant need for methods and materials for enhancing the attachment of HEK293 cells to solid substrates lacking an adlayer.

Current methods of culturing pluripotent stem cells, in particular, embryonic stem (ES) cells require complex culture conditions, such as, for example, culturing the embryonic stem cells on a solid substrate surface with a feeder cell layer, or on a solid substrate surface with an adlayer of extracellular matrix protein. Culture systems that employ these methods often use feeder cells or extracellular matrix proteins obtained from a different species than that of the stem cells being cultivated (xenogeneic material). Media obtained by exposure to feeder cells, that is, media conditioned by cells other than undifferentiated embryonic stem cells, may be used to culture the embryonic stem cells, and media may be supplemented with animal serum.

For example, Reubinoff et al. (Nature Biotechnol. 18:399-404, 2000) and Thompson et al. (Science 282:1145-1147, 1998) disclose the culture of ES cell lines from human blastocysts using a mouse embryonic fibroblast feeder cell layer.

In another example, Xu et al. (Nature Biotechnology 19:971-974, 2001) discloses the use of MATRIGEL® and laminin for treating solid substrate surfaces before feeder-cell free cultivation of human embryonic stem cells without differentiation.

In another example, Vallier et al. (J. Cell Sci. 118:4495-4509, 2005) discloses the use of fetal bovine serum for treating solid substrate surfaces before feeder-cell free cultivation of human embryonic stem cells without differentiation.

In another example, WO2005014799 discloses conditioned medium for the maintenance, proliferation and differentiation of mammalian cells. WO2005014799 state: “The culture medium produced in accordance with the present invention is conditioned by the cell secretion activity of murine cells, in particular, those differentiated and immortalized transgenic hepatocytes, named MMH (Met Murine Hepatocyte).”

In another example, Wanatabe et al. (Nature Biotechnol. 35:681-686, 2007) state “a ROCK inhibitor permits survival of dissociated human embryonic stem cells”, and demonstrate reduced dissociation-induced apoptosis, increases cloning efficiency (from approximately 1% to approximately 27%) and facilitation of subcloning after gene transfer, using mouse embryonic fibroblasts as feeder cells, collagen and MATRIGEL® as extracellular matrix protein, and Y-27632 or Fasudil for inhibition of ROCK. Furthermore, dissociated human embryonic stem cells treated with Y-27632 were protected from apoptosis in serum-free suspension culture.

In another example, Peerani et al. (EMBO Journal 26:4744-4755, 2007) state “Complexity in the spatial organization of human embryonic stem cell (hESC) cultures creates heterogeneous microenvironments (niches) that influence hESC fate. This study demonstrates that the rate and trajectory of hESC differentiation can be controlled by engineering hESC niche properties. Niche size and composition regulate the balance between differentiation-inducing and inhibiting factors. Mechanistically, a niche size-dependent spatial gradient of Smadl signaling is generated as a result of antagonistic interactions between hESCs and hESC-derived extra-embryonic endoderm (ExE). These interactions are mediated by the localized secretion of bone morphogenetic protein-2 (BMP2) by ExE and its antagonist, growth differentiation factor-3 (GDF3) by hESCs. Micropatterning of hESCs treated with small interfering (si) RNA against GDF3, BMP2 and Smad1, as well treatments with a Rho-associated kinase (ROCK) inhibitor demonstrate that independent control of Smad1 activation can rescue the colony size-dependent differentiation of hESCs. Our results illustrate, for the first time, a role for Smad1 in the integration of spatial information and in the niche-size dependent control of hESC self-renewal and differentiation.”

In another example, Koyanagi, M et al (J Neurosci Res. 2007 Sep. 7 [Epub ahead of print]) state “Rho-GTPase has been implicated in the apoptosis of many cell types, including neurons, but the mechanism by which it acts is not fully understood. Here, we investigate the roles of Rho and ROCK in apoptosis during transplantation of embryonic stem cell-derived neural precursor cells. We find that dissociation of neural precursors activates Rho and induces apoptosis. Treatment with the Rho inhibitor C3 exoenzyme and/or the ROCK inhibitor Y-27632 decreases the amount of dissociation-induced apoptosis (anoikis) by 20-30%. Membrane blebbing, which is an early morphological sign of apoptosis; cleavage of caspase-3; and release of cytochrome c from the mitochondria are also reduced by ROCK inhibition. These results suggest that dissociation of neural precursor cells elicits an intrinsic pathway of cell death that is at least partially mediated through the Rho/ROCK pathway. Moreover, in an animal transplantation model, inhibition of Rho and/or ROCK suppresses acute apoptosis of grafted cells. After transplantation, tumor necrosis factor-alpha and pro-nerve growth factor are strongly expressed around the graft. ROCK inhibition also suppresses apoptosis enhanced by these inflammatory cytokines. Taken together, these results indicate that inhibition of Rho/ROCK signaling may improve survival of grafted cells in cell replacement therapy.”

In another example, Yoneda et al (J. Cell Biol. 170: 443-453, Aug. 3, 2005) states “the homologous mammalian rho kinases (ROCK I and II) are assumed to be functionally redundant, based largely on kinase construct overexpression. As downstream effectors of Rho GTPases, their major substrates are myosin light chain and myosin phosphatase. Both kinases are implicated in microfilament bundle assembly and smooth muscle contractility. Here, analysis of fibroblast adhesion to fibronectin revealed that although ROCK II was more abundant, its activity was always lower than ROCK I. Specific reduction of ROCK I by siRNA resulted in loss of stress fibers and focal adhesions, despite persistent ROCK II and guanine triphosphate—bound RhoA. In contrast, the microfilament cytoskeleton was enhanced by ROCK II down-regulation. Phagocytic uptake of fibronectin-coated beads was strongly down-regulated in ROCK II—depleted cells but not those lacking ROCK I. These effects originated in part from distinct lipid-binding preferences of ROCK pleckstrin homology domains. ROCK II bound phosphatidylinositol 3,4,5P₃ and was sensitive to its levels, properties not shared by ROCK I. Therefore, endogenous ROCKs are distinctly regulated and in turn are involved with different myosin compartments.”

In another example, Harb et al (PloS ONE 3(8): e3001. oi:10.1371/journal.pone.0003001, August 2008) discloses an essential role of the Rho-Rock-Myosin signaling axis for the regulation of basic cell-cell communications in both mouse and human embryonic stem cells, and would contribute to advance [sic] in medically compatible xeno-free environments for human pluripotent stem cells.

The use of xenogeneic material may be unsuitable for certain applications utilizing pluripotent stem cells. Alternative materials may be used. For example, Stojkovic et al. (Stem Cells 23:895-902, 2005) discloses the use of human serum for treating solid substrate surfaces before feeder-cell free cultivation of human embryonic stem cells without differentiation.

An alternative culture system employs serum-free medium supplemented with growth factors capable of promoting the proliferation of embryonic stem cells.

For example, Cheon et al. (BioReprod DOI:10.1095/biolreprod.105.046870; 19 Oct. 2005) disclose a feeder-cell free, serum-free culture system in which ES cells are maintained in unconditioned serum replacement medium supplemented with different growth factors capable of triggering embryonic stem cell self-renewal.

In another example, Levenstein et al. (Stem Cells 24:568-574, 2006) disclose methods for the long-term culture of human embryonic stem cells in the absence of fibroblasts or conditioned medium, using media supplemented with basic fibroblast growth factor (FGF).

In another example, US20050148070 discloses a method of culturing human embryonic stem cells in defined media without serum and without fibroblast feeder cells, the method comprising: culturing the stem cells in a culture medium containing albumin, amino acids, vitamins, minerals, at least one transferrin or transferrin substitute, at least one insulin or insulin substitute, the culture medium essentially free of mammalian fetal serum and containing at least about 100 ng/ml of a FGF capable of activating a FGF signaling receptor, wherein the growth factor is supplied from a source other than just a fibroblast feeder layer, the medium supported the proliferation of stem cells in an undifferentiated state without feeder cells or conditioned medium.

In another example, US20050233446 discloses a defined media useful in culturing stem cells, including undifferentiated primate primordial stem cells. In solution, the media is substantially isotonic as compared to the stem cells being cultured. In a given culture, the particular medium comprises a base medium and an amount of each of basic FGF, insulin, and ascorbic acid necessary to support substantially undifferentiated growth of the primordial stem cells.

In another example, U.S. Pat. No. 6,800,480 states “In one embodiment, a cell culture medium for growing primate-derived primordial stem cells in a substantially undifferentiated state is provided which includes a low osmotic pressure, low endotoxin basic medium that is effective to support the growth of primate-derived primordial stem cells. The basic medium is combined with a nutrient serum effective to support the growth of primate-derived primordial stem cells and a substrate selected from the group consisting of feeder cells and an extracellular matrix component derived from feeder cells. The medium further includes nonessential amino acids, an anti-oxidant, and a first growth factor selected from the group consisting of nucleosides and a pyruvate salt.”

In another example, US20050244962 states: “In one aspect the invention provides a method of culturing primate embryonic stem cells. One cultures the stem cells in a culture essentially free of mammalian fetal serum (preferably also essentially free of any animal serum) and in the presence of fibroblast growth factor that is supplied from a source other than just a fibroblast feeder layer. In a preferred form, the fibroblast feeder layer, previously required to sustain a stem cell culture, is rendered unnecessary by the addition of sufficient fibroblast growth factor.”

In another example, WO2005065354 discloses a defined, isotonic culture medium that is essentially feeder-free and serum-free, comprising: a. a basal medium; b. an amount of basic fibroblast growth factor sufficient to support growth of substantially undifferentiated mammalian stem cells; c. an amount of insulin sufficient to support growth of substantially undifferentiated mammalian stem cells; and d. an amount of ascorbic acid sufficient to support growth of substantially undifferentiated mammalian stem cells.

In another example, WO2005086845 discloses a method for maintenance of an undifferentiated stem cell, said method comprising exposing a stem cell to a member of the transforming growth factor-beta (TGFβ) family of proteins, a member of the fibroblast growth factor (FGF) family of proteins, or nicotinamide (NIC) in an amount sufficient to maintain the cell in an undifferentiated state for a sufficient amount of time to achieve a desired result.

Pluripotent stem cells provide a potential resource for research and drug screening. At present, large-scale culturing of human embryonic stem cell lines is problematic and provides substantial challenges. A possible solution to these challenges is to passage and culture the human embryonic stem cells as single cells. Single cells are more amenable to standard tissue culture techniques, such as, for example, counting, transfection, and the like.

For example, Nicolas et al. provide a method for producing and expanding human embryonic stem cell lines from single cells that have been isolated by fluorescence-activated cell sorting following genetic modification by lentivirus vectors (Stem Cells Dev. 16:109-118, 2007).

In another example, US patent application US2005158852 discloses a method “for improving growth and survival of single human embryonic stem cells. The method includes the step of obtaining a single undifferentiated hES cell; mixing the single undifferentiated cell with an extracellular matrix to encompass the cell; and inoculating the mixture onto feeder cells with a nutrient medium in a growth environment.”

In another example, Sidhu et al. (Stem Cells Dev. 15:61-69, 2006) describe the first report of three human embryonic stem cell clones, hES 3.1, 3.2 and 3.3, derived from the parent line hES3 by sorting of single-cell preparations by flow cytometry.

However, passage and culture of human embryonic stem cells as single cells leads to genetic abnormalities and the loss of pluripotency. Culture conditions are important in the maintenance of pluripotency and genetic stability. Generally, passage of human embryonic stem cell lines is conducted manually or with enzymatic agents such as collagenase, LIBERSASE or DISPASE.

For example, Draper et al. note the presence of “karyotypic changes involving the gain of chromosome 17q in three independent human embryonic stem cell lines on five independent occasions.” (Nature Biotechnol. 22:53-54, 2004).

In another example, Buzzard et al. state, “we have only ever detected one karyotype change event . . . the culture methods used may have had some bearing on our results, given that our methods are distinctly different from those used by most other groups.

Typically we passage human ES cells after 7 days by first dissecting the colony with the edge of a broken pipette . . . . No enzymatic or chemical methods of cell dissociation are incorporated into this method. We speculate that this may explain the relative cytogenetic resilience of hES (human ES) cells in our hands.” (Nature Biotechnol. 22:381-382, 2004).

In another example, Mitalipova et al. state “bulk passage methods . . . can perpetuate aneuploid cell populations after extended passage in culture, but may be used for shorter periods (up to at least 15 passages) without compromising the karyotypes . . . it may be possible to maintain a normal karyotype in hES cells under long-term manual propagation conditions followed by limited bulk passaging in experiments requiring greater quantities of hES cells than manual passage methods, alone, can provide”. (Nature Biotechnol. 23:19-20, 2005).

In another example, Heng et al. state “the results demonstrated that the second protocol (trypsinization with gentle pipetting) is much less detrimental to cellular viability than is the first protocol (collagenase treatment with scratching). This in turn translated to higher freeze-thaw survival rates.” (Biotechnology and Applied Biochemistry 47:33-37, 2007).

In another example, Hasegawa et al. state “we have established hESC sublines tolerant of complete dissociation. These cells exhibit high replating efficiency and also high cloning efficiency and they maintain their ability to differentiate into the three germ layers.” (Stem Cells 24:2649-2660, 2006).

Therefore, there is a significant need for methods and compositions for the cultivation of mammalian cells, including cultivation of pluripotent stem cells in the absence of feeder cells and an adlayer, while maintaining the pluripotency of the cells.

### SUMMARY

In one embodiment, the present invention provides methods and compositions for the attachment, cultivation and detachment of cells to a solid substrate surface containing from at least about 0.5% N, a sum of O and N of greater than or equal to 17.2% and a contact angle of at least about 13.9 degrees, lacking a feeder cell layer and lacking an adlayer.

In one embodiment, the present invention provides a method to enhance the attachment of cells to a surface containing from at least about 0.5% N, a sum of O and N of greater than or equal to 17.2% and a contact angle of at least about 13.9 degrees, lacking a feeder cell layer and lacking an adlayer, comprising the steps of:
a. Obtaining a suspension of cells,
b. Treating the suspension of cells with at least one compound selected from the group consisting of: a compound capable of inhibiting Rho kinase activity, and a compound capable of inhibiting Rho activity, and
c. Adding the suspension of cells to the surface and allowing the cells to attach.

In one embodiment, the cells are maintained in culture after the cells attach to the surface. In an alternate embodiment the at least one compound is removed.

In one embodiment, the cells are detached from the surface by removing the at least one compound.

In one embodiment the suspension of cells is a suspension of clusters of cells. In an alternate embodiment, the suspension of cells is a suspension of single cells.

In one embodiment, the cells are pluripotent stem cells. In an alternate embodiment, the cells are stem cells.

In one embodiment, the present invention provides a method to enhance the attachment of cells to a surface containing from at least about 0.9% N, a sum of O and N of greater than or equal to 22.3% and a contact angle of at least about 13.9 degrees, lacking a feeder cell layer and lacking an adlayer, comprising the steps of:
a. Obtaining a suspension of cells, and
b. Adding the suspension of cells to the surface and allowing the cells to attach.

### BRIEF DESCRIPTION OF THE DRAWINGS

FIG. 1 shows phase contrast micrographs (4×) of cells of the human embryonic stem cell line H1 that were passaged twice as clusters with LIBERASE on either surface modified plates 2, 3 or 4. Images of cells of the human embryonic stem cell line H1, cultured on plates treated with a 1:30 dilution of MATRIGEL®, NUNCLON DELTA™ plates are also shown.

FIG. 2 shows the effect of 10 μM Y-27632 on the attachment of human embryonic stem cells to the surface modified plates of the present invention. The figure shows phase contrast micrographs (4×) of cells of the human embryonic stem cell line H1 that were passaged twice as clusters on either surface modified plates 3 or 4. Cells were then passaged onto either surface modified plates 2, 3 or 4, in MEF conditioned medium containing 10 μM Y-27632. Cells were cultured for four days prior to taking the photographs. Cells cultured in the absence of Y-27632 were included as controls.

FIG. 3 shows a schematic of the time-course of treatment of compounds on human embryonic stem cells cultured on the surface modified plates of the present invention. Cells of the human embryonic stem cell line H1 were passaged four times as clusters with LIBERASE treatment on either surface modified plates 3, or 4, and cultured in MEF conditioned medium. Cells were treated for the first two days after passage with either 10 μM of the Rho Kinase inhibitor, Y-27632, or with 0.5 ng/ml of the Rho inhibitor, a cell permeable form of exoenzyme C3 transferase. Cells that were treated with the Rho Kinase inhibitor, Y-27632 and were thereafter treated for the first two days after each passage with Y-27632 on surface modified plate 3 are referred to as “7s”. Cells that were treated with the Rho Kinase inhibitor, Y-27632 and were thereafter treated for the first two days after each passage with Y-27632 on surface modified plate 4 are referred to as “3s”. Cells that were treated with the Rho inhibitor for two days and were then treated with the Rho Kinase inhibitor, Y-27632 for two days after each passage and thereafter treated for the first two days after passage with Y-27632 on surface modified plate 3 are referred to as “5s”. Cells that were treated with the Rho inhibitor for two days and were then treated with the Rho kinase inhibitor, Y-27632 for two days after each passage and thereafter treated for the first two days after passage with Y-27632 on surface modified plate 4 are referred to as “1s”.

FIG. 4 shows the expression of markers associated with pluripotency and differentiation in human embryonic stem cells as determined by qRT-PCR.

FIG. 5 shows the expression of pluripotency markers in cells of the human embryonic stem cell line H1 as determined by flow cytometry at passage 4 (p4), passage 9 (p9), and again at passage 10, 11, or 12 (p10, p11, or p12).

FIG. 6 shows immuno-fluorescent images of cells of the human embryonic stem cell line H1 were passaged serially as clusters with LIBERASE treatment on surface modified plate 4, and cultured in MEF conditioned medium. Expression of proteins associated with markers of pluripotency was detected in cells cultured for 11 passages on surface modified plate 4. Cells were treated with 10 μM Y-27632 for two days after each passage.

FIG. 7 shows the ability for human embryonic stem cells to form definitive endoderm after culture on the surface modified plates of the present invention. Cells of the human embryonic stem cell line H1 were passaged 11 times as clusters with LIBERASE treatment on either surface modified plates 3, or 4 and cultured in MEF conditioned medium. At passage 8 (p8) and again at passage 10 or 11 (p10-11) cells were treated with DMEM:F12 media containing 0.5% FBS, 100 ng/ml Activin A, and 20 ng/ml Wnt3a for two days and then treated with DMEM:F12 media containing 2% FBS and 100 ng/ml Activin A for three more days. The y-axis on the graph shows the percent positive CXCR4 cells obtained by flow cytometry. See also Table 5.

FIG. 8 shows the ability for human embryonic stem cells to form pancreatic endoderm after culture on the surface modified plates of the present invention. Cells of the human embryonic stem cell line H1 were passaged eight times as clusters with LIBERASE treatment on either surface modified plates 3, or 4 and cultured in MEF conditioned medium. At passage 8 (p8) cells were subjected to differentiation to definitive endoderm by treatment with DMEM:F12 media containing 0.5% FBS, 100 ng/ml Activin A, and 20 ng/ml Wnt3a for two days and then treated with DMEM:F12 media containing 2% FBS and 100 ng/ml Activin A for three more days. The cells were then further differentiated to embryonic foregut with four days of treatment with DMEM:F12 media containing 2% FBS, 100 ng/ml FGF-10, and 1 μM cyclopamine-KAAD. The cells were then differentiated to pancreatic endoderm with four days of treatment with DMEM:F12 media containing 1% B-27, 100 ng/ml FGF-10, 1 μM cyclopamine-KAAD and 2 μM retinoic acid. Cells were stained by immunofluorescence for PDX-1 (green) and E-cadherin (red) and total cell number was identified by Hoechst dye (blue).

FIG. 9 shows the ability of human embryonic stem cells cultured on the surface modified plates of the present invention to form embryoid bodies.

FIG. 10 shows the karyotype of human embryonic stem cells cultured on surface modified plate 4.

FIG. 11 shows the effect of treatment with Rho kinase inhibitors (Y-27632 from EMD biosciences, Y-27632 from Sigma, Fasudil, and Hydroxyfasudil) on the attachment of human embryonic stem cells to the surface modified plates of the present invention. Cells were cultured in medium containing the indicated compounds, at the concentrations listed, for three days. Cells were stained with crystal violet and images taken.

FIG. 12 shows the dose-response of Y-27632 on the attachment of human embryonic stem cells to the surface modified plates of the present invention. Various concentrations of the Rho kinase inhibitor, Y-27632, was added to the cultures at a specified concentration (0, 1, 2, 4, or 10 μM Y-27632) for the first day. The cells were then maintained from day 2 onward in media containing 10 μM Y-27632 with daily media changes for five days. Media was removed from the plates on day five and the cells were stained with 0.5% crystal violet, and images taken.

FIG. 13 shows the formation of human embryonic stem cell colonies four days after passage onto either surface modified plates 2, 3, or 4 with or without 10 of μM Y-27632.

FIG. 14 shows the formation of human embryonic stem cell colonies four days after passage onto MATRIGEL® treated plates, either with or without 10 μM Y-27632.

FIG. 15 shows the difference between continual and intermittent treatment of human embryonic stem cells with Y-27632, on attachment of cells to the surface modified plates of the present invention.

FIG. 16 depicts images of cells from the human embryonic stem cell line H9, that were passaged as single cells, seeded on to surface modified plate 3 in MEF conditioned media containing (panel B) or without (panel A) 10 μM Y-27632. The images were taken 24 hours after seeding.

FIG. 17 depicts the expression of markers associated with pluripotency in cells from the human embryonic stem cell line H9, that were passaged as single cells for 5 passages, using TrypLE™ Express, and plated onto either surface modified plates 3 or 4, with or without 10 μM of Y-27632 (Y). The pluripotency markers are listed on the x-axis and the percentage of positive cells is shown on the y-axis.

FIG. 18 depicts the total cell number of cells from the human embryonic stem cell line H9, that were passaged as single cells, plated onto either surface modified plates 3 or 4. The effect of 10 μM of Y-27632 (Y) on cell number was examined on cells passaged on MATRIGEL® (naïve, N), and cells passaged 10 times on the surface modified plates (acclimated, A). The different cell conditions are listed on the x-axis and the number of cells divided by 10⁴ is shown in the y-axis.

FIG. 19 depicts the rate of growth of cells from the human embryonic stem cell line H9, that were passaged as single cells on MATRIGEL® treated plates prior to the study. Cells were seeded at 10⁴ cells/cm² and cultured in MEF conditioned media with or without 10 μM of Y-27632 on either surface modified plates 3 or 4. The y-axis shows the number of cells collected 2, 3 or 4 days after seeding (divided by 10⁴).

FIG. 20 depicts the rate of growth of cells from the human embryonic stem cell line H9, that were passaged as single cells for 10 passages on surface modified plates prior to the study. Cells were seeded at 10⁴ cells/cm² and cultured in MEF conditioned media with or without 10 μM of Y-27632 on either surface modified plates 3 or 4. The y-axis shows the number of cells collected 2, 3 or 4 days after seeding (divided by 10⁴).

FIG. 21 depicts images of cells from the human embryonic stem cell line H9, that were passaged as single cells, seeded on to either surface modified plates 2, 3, 4 or 13 in a 96-well format. The MEF conditioned media contained 10 μM of Y-27632. Images were taken 48 hours after seeding.

FIG. 22 shows the ability of cells from the human embryonic stem cell line H9, that were passaged as single cells, seeded on to either surface modified plates 3 or 4 to differentiate into definitive endoderm. The extent of formation of definitive endoderm was determined by measuring CXCR expression by flow cytometry. The effect of 10 μM Y-27632 on the formation of definitive endoderm was investigated. Cells were treated with Y-27632 during expansion. Cells expanded and differentiated on MATRIGEL® were included as a control. The y-axis shows percent positive CXCR4 cells obtained by flow cytometry.

FIG. 23 shows the ability of cells from the human embryonic stem cell line H9, that were passaged as single cells, seeded on to either surface modified plates 3 or 4 to differentiate into pancreatic endoderm. Cells were plated onto the surface modified plates and cultured in MEF conditioned medium containing 10 μM Y-27632, and passaged 8 times on the surface modified plates prior to differentiation. The y-axis shows the fold increase of pancreatic differentiation marker expression (Ngn3, Pdx1, Insulin) by q-PCR at the posterior foregut stage (PF) and the hormone expressing endocrine cell stage (EN).

FIG. 24 shows the attachment of human embryonic stem cells to surface modified plates. Passage 50 H9 human embryonic stem cells were plated at a 1:2 dilution on either Surfaces 3, 4, CELLBIND™, or PRIMARIA™. Media was removed from the plates 24 hours after plating and the cells were stained with 0.5% crystal violet, and images taken. Arrows indicate colonies.

FIG. 25 shows the attachment of human embryonic stem cells to surface modified plates. Passage 50 H9 human embryonic stem cells were plated at a 1:2 dilution on either Surfaces 3, 4, CELLBIND™, or PRIMARIA™ in the presence of various concentrations of Y-27632 (0, 1, 2, 4, 10 and 20 micromolar). Media was removed from the plates 24 hours after plating and the cells were stained with 0.5% crystal violet, and images taken. Colonies are dark spots on the well. Arrows are used to highlight colonies on the untreated wells.

FIG. 26 shows the attachment of human embryonic stem cells to surface modified plates. Passage 53 H9 human embryonic stem cells were plated at a 1:3 dilution on either Surfaces 2, 3, 4, 13, CELLBIND™, or PRIMARIA™ in the absence or presence of Y-27632 (0 or 20 micromolar). Media was removed from the plates 48 hours after plating and the cells were stained with 0.5% crystal violet, and images taken. Colonies are dark spots on the well. Arrows are used to highlight colonies on the untreated wells.

FIG. 27 shows the first attempt (October) and second attempt (December) to attach human H9 embryonic stem cells to either surface modified plates 14 or 15 and an attempt to attach human H1 embryonic stem cells to either surface modified plates 14 or 15. Passage 42 and passage 53 H9 human embryonic stem cells, and passage 57 H1 human embryonic stem cells were plated at a 1:2 or 1:3 dilution to the modified surfaces in the presence of 20 micromolar Y-27632. Media was removed from the plates 24-48 hours after plating and the cells were stained with 0.5% crystal violet, and images taken. Colonies are dark spots on the well. Arrows are used to highlight colonies on the plates.

FIG. 28 shows the attachment of human embryonic stem cells to surface modified plate 4 in defined media, mTeSR™. Passage 50 H9 human embryonic stem cells were plated at a 1:2 dilution to the modified surfaces in the absence or presence of Y-27632 (0 or 20 micromolar) in wells that were untreated or treated with proteins (0.1% gelatin, 2% BSA, 0.34 mg/ml rat Collagen 1, 1:1000 diluted MATRIGEL®, or 1:5000 diluted MATRIGEL®). Media was removed from the plates 48 hours after plating and the cells were stained with 0.5% crystal violet, and images taken. Colonies are dark spots on the well.

FIG. 29 shows the water contact angles of the surface modified plates of the present invention, measured over 11 weeks using the static sessile drop method. The first measurement was done one week after surface treatment and sterilization. Each data point represents the mean contact angle (one measurement on each of 7 drops). The contact angles on NUNCLON DELTA™ and CELLBND™ plates were measured under the same experimental conditions as Surfaces 1-4 and 13, but the surface treatment and sterilization was done more than 12 weeks before the first measurement (NUNCLON DELTA™* was sterilized one week before the first measurement).

FIG. 30 shows the density of negative charges on the surface modified plates of the present invention measured as reactivity of surfaces with positively charged crystal violet. Three samples of each surface were tested, and absorbance measurements on desorbed crystal violet from each sample were performed in triplicate. Mean and standard deviation of nine measurements are given.

FIG. 31 shows the effect of solid substrate surfaces and Y-27632 on attachment and growth of HEK293 cells in chemically defined, serum-free Pro293a-CDM™ medium (A) or EMEM medium supplemented with 10% fetal bovine serum (B). HEK293 cells were seeded in 96-well plates with either CELLBIND™ surfaces, NUNCLON DELTA™ surfaces, or Surface 4. The number of HEK293 cells attached to these surfaces is shown as a function of culture conditions and concentration of Y-27632. Cells received either: (i) 96 hours of constant treatment in culture with Y-27632 (Y-27632 96 h on); or (ii) 48 hours of constant treatment in culture with Y-27632 followed by a change of medium and then 48 hours in culture without Y-27632 (Y-27632 48 h on/48 h off). HEK293 cells cultured without Y-27632 in the medium (No Y-27632) were handled the same way as cells cultured with Y-27632, that is, for either 96-hours without a change of medium, or with a change of medium after 48 hours. Y-27632 enhanced attachment of HEK293 cells on Surface 4 and the CELLBIND™ surface when applied at concentrations of 2.0 and 5.0 μM. Removing Y-27632 after 48 hours of incubation resulted in detachment of a significant number of cells from Surface 4 and the CELLBIND™ surface. Mean and standard deviation of three measurements are shown.

FIG. 32 shows the effect of solid substrate surfaces and Rho kinase inhibitors Y-27632 and H-1152 on growth of HEK293 cells in EMEM medium supplemented with 10% fetal bovine serum. HEK293 cells were seeded in Multidish 24-well plates with either Surface 4 (A) or a non-treated (but gamma irradiated; 25 kGy) polystyrene surface (B).

FIG. 33 shows the effect of H-1152 and Surface 4 on HEK293-cell attachment and morphology. HEK293 cells were seeded in Multidish 12-well plates in EMEM medium supplemented with 10% fetal bovine serum and H-1152, and incubated for 67 hours in an automated, in-incubator microscope. Growth curves in A and photomicrographs in B show the general effect of H-1152 on HEK293-cell attachment and growth on surface 4, and the effect of a change of medium on HEK293-cell attachment and morphology on surface 4 in the presence or absence of H-1152.

FIG. 34 shows growth curves over 3 passages for HEK293 cells grown on surface 4 and NUNCLON DELTA™ surface in the absence or presence of 2.5 μM Y-27632. HEK293 cells in EMEM medium supplemented with 10% fetal bovine serum were passaged 3 times by trypsinization.

FIG. 35 shows the effect of Rho kinase inhibition on the attachment of cells of the human embryonic stem cell line H1 to either surface modified plates 4, 18 ,19, or PRIMARIA™. Wells A & B were control wells on all plates. Wells C&D contained 10 μM Y-27632. Wells E & F contained 3 μM H1152-glycyl. Wells G & H contained 10 μM H1152-glycyl.

FIG. 36 shows the effect of Rho kinase inhibition on the attachment of cells of the human embryonic stem cell line H1 to surface modified plate 30. (--)=no treatment. (RI)=3 μM H1152-glycyl. (MG)=adlayer of 1:30 dilution of MATRIGEL®. (MG+RI)=adlayer of 1:30 dilution of MATRIGEL®+3 μM H1152-glycyl.

FIG. 37 shows the effect of Rho kinase inhibition on the attachment of cells of the human embryonic stem cell line H1 to surface modified plate 31. (--)=no treatment. (RI)=3 μM H1152-glycyl. (MG)=adlayer of 1:30 dilution of MATRIGEL®. (MG+RI)=adlayer of 1:30 dilution of MATRIGEL®+3 μM H1152-glycyl.

FIG. 38 shows the effect of Rho kinase inhibition on the attachment of cells of the human embryonic stem cell line H1 to surface modified plate 32. (--)=no treatment. (RI)=3 μM H1152-glycyl. (MG)=adlayer of 1:30 dilution of MATRIGEL®. (MG+RI)=adlayer of 1:30 dilution of MATRIGEL®+3 μM H1152-glycyl.

FIG. 39 shows the effect of Rho kinase inhibition on the attachment of cells of the human embryonic stem cell line H1 to surface modified plate 33. (--)=no treatment. (RI)=3 μM H1152-glycyl. (MG)=adlayer of 1:30 dilution of MATRIGEL®. (MG+RI)=adlayer of 1:30 dilution of MATRIGEL®+3 μM H1152-glycyl.

FIG. 40 shows the effect of Rho kinase inhibition on the attachment of cells of the human embryonic stem cell line H1 to surface modified plate 34. (--)=no treatment. (RI)=3 μM H1152-glycyl. (MG)=adlayer of 1:30 dilution of MATRIGEL®. (MG+RI)=adlayer of 1:30 dilution of MATRIGEL®+3 μM H1152-glycyl.

FIG. 41 shows the water contact angles of the surface modified plates of the present invention, measured over 40 weeks using the static sessile drop method.

FIG. 42 shows the water contact angles of the surface modified plates of the present invention, measured using the static sessile drop method.

FIG. 43 shows the density of negative charges on the surface modified plates of the present invention, measured as reactivity of surfaces with positively charged crystal violet.

FIG. 44 shows the density of negative charges on surface modified plates 4, 22-24 and 29 measured as reactivity of surfaces with positively charged crystal violet. Three samples of each surface were tested, and absorbance measurements on desorbed crystal violet from each sample were performed in triplicate. The negative charge density for surfaces 4, 22-24 and 29 was normalized to the negative charge density of the NUNCLON DELTA™ surface. Mean and standard deviation of nine measurements are given.

FIG. 45 shows the growth and attachment of pluripotent stem cells derived from amniotic fluid-derived cells to a surface that lacks a feeder-cell layer and an adlayer.

FIG. 46 shows the expression of genes associated with pluripotency in pluripotent stem cells derived from amniotic fluid-derived cells that have been cultured on a surface that lacks a feeder-cell layer and an adlayer.

FIG. 47 shows the expression of markers characteristic of the definitive endoderm lineage in a populations of pluripotent stem cells derived from amniotic fluid-derived cells that have been cultured on a surface that lacks a feeder-cell layer and an adlayer.

FIG. 48 panel a is an H&E stained micrograph of a graft of cells of the human embryonic stem cell line H1 implanted under the kidney capsule of a SCID mouse. Panel b is another H&E stained micrograph of a graft of cells of the human embryonic stem cell line H1 implanted under the kidney capsule of a SCID mouse. The arrowheads denote cells of the germ layer identified in each of the panels.

FIG. 49 shows the expression of ectodermal markers (nestin and MAP-2) and the mesodermal marker (cardiac tropinin) in cells of the human embryonic stem cell line H1 implanted under the kidney capsule of a SCID mouse.

FIG. 50 panel A shows the shows the expression of markers associated with pluripotency in cells of the human embryonic stem cell line H1 cultured on surface modified plate 4 in the conditions indicated. Panel B shows micrographs of the cells of the human embryonic stem cell line H1 cultured on surface modified plate 4.

### DETAILED DESCRIPTION

For clarity of disclosure, and not by way of limitation, the detailed description of the invention is divided into the following subsections that describe or illustrate certain features, embodiments or applications of the present invention.

### DEFINITIONS

“Adlayer” as used herein refers to a layer that is formed on a surface of a solid substrate, by attaching molecules to the surface by either covalent (also known as grafting) or non-covalent (also known as adsorption) bonds. Molecules used in making an adlayer can, for example, be proteinaceous molecules, which may include, for example, extracellular matrix proteins, amino acids and the like, and non-biological molecules, such as, for example, polyethyleneimine.

“β-cell lineage” refers to cells with positive gene expression for the transcription factor PDX-1 and at least one of the following transcription factors: NGN3, NKX2.2, NKX6.1, NEUROD, ISL-1, HNF3 beta, MAFA, PAX4, or PAX6. Cells expressing markers characteristic of the 13 cell lineage include 13 cells.

“Cells expressing markers characteristic of the definitive endoderm lineage” as used herein refers to cells expressing at least one of the following markers: SOX17, GATA4, HNF3 beta, GSC, CER™, Nodal, FGF8, Brachyury, Mix-like homeobox protein, FGF4 CD48, eomesodermin (EOMES), DKK4, FGF17, GATA6, CXCR4, C-Kit, CD99, or OTX2. Cells expressing markers characteristic of the definitive endoderm lineage include primitive streak precursor cells, primitive streak cells, mesendoderm cells and definitive endoderm cells.

“Cells expressing markers characteristic of the pancreatic endoderm lineage” as used herein refers to cells expressing at least one of the following markers: PDX1, HNF1 beta, PTF1 alpha, HNF-6, or HB9. Cells expressing markers characteristic of the pancreatic endoderm lineage include pancreatic endoderm cells.

“Cells expressing markers characteristic of the pancreatic endocrine lineage” as used herein refers to cells expressing at least one of the following markers: NGN3, NEUROD, ISL-1, PDX1, NKX6.1, PAX4, NGN3, or PTF1 alpha. Cells expressing markers characteristic of the pancreatic endocrine lineage include pancreatic endocrine cells, pancreatic hormone expressing cells, and pancreatic hormone secreting cells, and cells of the β-cell lineage.

“Definitive endoderm” as used herein refers to cells which bear the characteristics of cells arising from the epiblast during gastrulation and which form the gastrointestinal tract and its derivatives. Definitive endoderm cells express the following markers: CXCR4, HNF3 beta, GATA4, SOX-17, Cerberus, OTX2, goosecoid, c-Kit, CD99, and MIXL1.

“Extracellular matrix proteins” refers to proteinaceous molecules normally found between cells in the body or in the placenta. Extracellular matrix proteins can be derived from tissue, body fluids, such as, for example, blood, or media conditioned by non-recombinant cells or recombinant cells or bacteria.

“Extraembryonic endoderm” as used herein refers to a population of cells expressing at least one of the following markers: SOX7, AFP, or SPARC.

“HEK293 cells” refers to a cell line generated by transformation of a culture of normal human embryonic kidney cells as described by Graham et al. (J. Gen. Virol. 36:59-72, 1977), and any cells derived from this parent cell line.

“Markers” as used herein, are nucleic acid or polypeptide molecules that are differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

“Matrix” as used herein refers to a 3-dimensional support to which cells may attach.

“Mesendoderm cell” as used herein refers to a cell expressing at least one of the following markers: CD48, eomesodermin (EOMES), SOX17, DKK4, HNF3 beta, GSC, FGF17, or GATA6.

“Pancreatic endocrine cell” or “pancreatic hormone expressing cell” as used herein refers to a cell capable of expressing at least one of the following hormones: insulin, glucagon, somatostatin, or pancreatic polypeptide.

“Pancreatic hormone secreting cell” as used herein refers to a cell capable of secreting at least one of the following hormones: insulin, glucagon, somatostatin, or pancreatic polypeptide.

“Pre-primitive streak cell” as used herein refers to a cell expressing at least one of the following markers: Nodal, or FGF-8.

“Primitive streak cell” as used herein refers to a cell expressing at least one of the following markers: Brachyury, Mix-like homeobox protein, or FGF4.

“Surface” as used herein refers to the outermost layer of molecules of a solid substrate vessel or matrix intended for use in cell culture or analysis. The elemental composition, the roughness, and the wettability of the surface can be analyzed by X-Ray Photoelectron Spectroscopy (XPS), Atomic Force Microscopy (AFM), and contact angle measurement, respectively.

“Surface modified plate” refers to a vessel containing any one of surfaces 1-34, described in Examples 16, 17 and 26, or plates containing surfaces that are sold under the trade names NUNCLON DELTA™, COSTAR™, FALCON™, CELLBIND™, and PRIMARIA™. The vessel can, for example, be made of a polymer, such as polystyrene (PS), cyclic olefin copolymer (COC), polycarbonate (PC), polymethyl methacrylate (PMMA), or styrene acrylonitrile copolymer (SAN).

Stem cells are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (i) totipotent, meaning able to give rise to all embryonic and extraembryonic cell types; (ii) pluripotent, meaning able to give rise to all embryonic cell types; (iii) multipotent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (iv) oligopotent, meaning able to give rise to a more restricted subset of cell lineages than multipotent stem cells; and (v) unipotent, meaning able to give rise to a single cell lineage (e.g., spermatogenic stem cells).

Differentiation is the process by which an unspecialized (“uncommitted”) or less specialized cell acquires the features of a specialized cell such as, for example, a nerve cell or a muscle cell. A differentiated or differentiation-induced cell is one that has taken on a more specialized (“committed”) position within the lineage of a cell. The term committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. Dedifferentiation refers to the process by which a cell reverts to a less specialized (or committed) position within the lineage of a cell. As used herein, the lineage of a cell defines the heredity of the cell, that is, which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

Various terms are used to describe cells in culture. “Maintenance” refers generally to cells placed in a growth medium under conditions that facilitate cell growth and/or division that may or may not result in a larger population of the cells. “Passaging” refers to the process of removing the cells from one culture vessel and placing them in a second culture vessel under conditions that facilitate cell growth and/or division.

A specific population of cells, or a cell line, is sometimes referred to or characterized by the number of times it has been passaged. For example, a cultured cell population that has been passaged ten times may be referred to as a P10 culture. The primary culture, that is, the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (that is, the number of population doublings) during the period between passaging depends on many factors, including but not limited to the seeding density, substrate, medium, growth conditions, and time between passaging.

In one embodiment, the present invention provides a method to enhance the attachment of cells to a surface containing from at least about 0.9% N, a sum of O and N of greater than or equal to 22.3% and a contact angle of at least about 13.9 degrees, lacking a feeder cell layer and lacking an adlayer, comprising the steps of:
a. Obtaining a suspension of cells, and
b. Adding the suspension of cells to the surface and allowing the cells to attach.

In one embodiment, the present invention provides a method to enhance the attachment of cells to a surface containing from at least about 0.5% N, a sum of O and N of greater than or equal to 17.2% and a contact angle of at least about 13.9 degrees, lacking a feeder cell layer and lacking an adlayer, comprising the steps of:
a. Obtaining a suspension of cells,
b. Treating the suspension of cells with at least one compound selected from the group consisting of: a compound capable of inhibiting Rho kinase activity, and a compound capable of inhibiting Rho activity, and
c. Adding the suspension of cells to the surface and allowing the cells to attach.

In one embodiment the suspension of cells is a suspension of clusters of cells. In an alternate embodiment, the suspension of cells is a suspension of single cells.

In one embodiment, the cells are pluripotent stem cells. In an alternate embodiment, the cells are stem cells.

In one embodiment, the surface has an adlayer. In one embodiment, the adlayer is an extracellular matrix component, such as, for example, those derived from basement membrane or that may form part of adhesion molecule receptor-ligand couplings. In one embodiment, the adlayer is made from MATRIGEL® (Becton Dickenson). MATRIGEL® is a soluble preparation from Engelbreth-Holm Swarm tumor cells that gels at room temperature to form a reconstituted basement membrane. The proteinaceous adlayer may also be formed from laminin, fibronectin, proteoglycan, entactin, heparan sulfate, and the like, alone or in various combinations.

In one embodiment, the cells are maintained in culture after the cells attach to the surface. In an alternate embodiment the at least one compound is removed after the cells attach to the surface. In one embodiment, the cells are detached from the surface by removing the at least one compound.

In one embodiment, the suspension of cells is treated with at least one compound capable of inhibiting Rho kinase activity. In an alternate embodiment, the suspension of cells is treated with at least one compound capable of inhibiting Rho activity. In an alternate embodiment, the suspension of cells is treated with at least one compound capable of inhibiting Rho kinase activity and at least one compound capable of inhibiting Rho activity.

The at least one compound capable of inhibiting Rho kinase activity is selected from the group consisting of: Y-27632, Fasudil, and Hydroxyfasudil.

In one embodiment, the at least compound capable of inhibiting Rho kinase activity is Y-27632.

The at least one compound capable of inhibiting Rho kinase activity may be used at a concentration from about 0.1 μM to about 100 μM. In one embodiment, the at least one compound capable of inhibiting Rho kinase activity is used at a concentration of about 10 μM.

In one embodiment, the at least one compound capable of inhibiting Rho activity is a Rho GTPase inhibitor.

In one embodiment, the at least one compound capable of inhibiting Rho activity is exoenzyme C3 Transferase.

The at least one compound capable of inhibiting Rho activity may be used at a concentration from about 0.01 μg/ml to about 5 μg/ml. In one embodiment, the at least one compound capable of inhibiting Rho activity is used at a concentration of about 0.5 μg/ml.

### Surface Modified Plates

Surface modified plates suitable for use in the present invention may be vessels whose surfaces have been modified to contain from at least about 0.5% N, a sum of O and N of greater than or equal to 17.2% and a contact angle of at least about 13.9 degrees. Alternatively, the surface may be a 3-dimensional matrix, such as, for example, a porous scaffold, to which cells can attach.

In one embodiment, the surface modified plate comprises a plate whose surface contains from at least about 0.5% N, a sum of O and N of greater than or equal to 17.2% and a contact angle of at least about 13.9 degrees. In an alternate embodiment, the surface modified plate comprises a plate whose surface contains from at least about 0.5% N, a sum of O and N of greater than or equal to 19.5% and a contact angle of at least about 13.9 degrees.

In one embodiment, the surface modified plate comprises a plate whose surface contains from at least about 1.3% N, a sum of O and N of at least about 24.9% and a contact angle of at least about 20.7 degrees, which is referred herein as surface modified plate 1.

In one embodiment, the surface modified plate comprises a plate whose surface contains from at least about 1.7% N, a sum of O and N of at least about 29.6% and a contact angle of at least about 14.3 degrees, which is referred herein as surface modified plate 2.

In one embodiment, the surface modified plate comprises a plate whose surface contains from at least about 2.0% N, a sum of O and N of at least about 30.7% and a contact angle of at least about 18.4 degrees, which is referred herein as surface modified plate 3.

In one embodiment, the surface modified plate comprises a plate whose surface contains from at least about 2.1% N, a sum of O and N of at least about 30.2% and a contact angle of at least about 17.4 degrees, which is referred herein as surface modified plate 4.

In one embodiment, the surface modified plate comprises a plate whose surface contains from at least about 1.8% N, a sum of O and N of at least about 28.2% and a contact angle of at least about 18.8 degrees, which is referred herein as surface modified plate 13.

In one embodiment, the surface modified plate comprises a plate whose surface contains from at least about 1.0% N, a sum of O and N of at least about 27.8% and a contact angle of at least about 44.3 degrees, which is sold under the trade name CELLBIND.

In one embodiment, the surface modified plate comprises a plate whose surface contains from at least about 10.2% N, a sum of O and N of at least about 23.0% and a contact angle of at least about 39.5 degrees, which is sold under the trade name PRIMARIA.

### Characterization of the Surface Modified Plates

In one embodiment, the elemental composition of the surface of the surface modified plates may be analyzed by X-Ray Photoelectron Spectroscopy (XPS). XPS, also known as Electron Spectroscopy for Chemical Analysis (ESCA), is used as a method to determine what elements or atoms are present in the surface of a solid substrate (all elements in concentrations less than 0.1 atomic percent can be detected, except hydrogen and helium), and to determine the bonding environment of such elements or atoms. As an example, an XPS analysis of a polystyrene (contains only carbon and hydrogen) solid sample would typically give greater than 97% carbon, less than 3% oxygen, and 0% nitrogen (hydrogen is not detected; different levels of oxygen may be detected due to oxidation of the polystyrene chains at the surface, for example, as a result of sterilization by irradiation) (Brevig et al., Biomaterials 26:3039-3053, 2005; Shen and Horbett, J. Biomed. Mater. Res. 57:336-345, 2001).

In one embodiment, the roughness of the surface of the surface modified plates may be analyzed by Atomic Force Microscopy (AFM). Surface atoms or molecules with a lateral resolution down to 1 Å and a vertical resolution down to 0.1 Å can be imaged by AFM.

In one embodiment, the wettability of the surface of the surface modified plates may be analyzed by measuring the contact angle. For example, contact angle measurement by the static sessile drop method provides information on the interaction between the surface of a solid substrate and a liquid. The contact angle describes the shape of a liquid drop resting on the surface of the solid substrate, and is the angle of contact of the liquid on the surface of the solid substrate, measured within the liquid at the contact line where liquid, solid, and gas meet. A surface with a water contact angle larger than 90° is termed hydrophobic, and a surface with water contact angle less than 90° is termed hydrophilic. On extremely hydrophilic surfaces, that is, surfaces that have a high affinity for water, a water droplet will completely spread (an effective contact angle of 0°.

In one embodiment, the negative charge density of the surface of the surface modified plates may be analyzed by measuring the reactivity of the surface with crystal violet. Crystal violet carries a positive charge, which enables it to bind to negatively charged molecules and parts of molecules, for example, negatively charged functional groups present on a polymer surface. A surface with a high crystal violet reactivity has a higher density of negative charges than a surface with a low crystal violet reactivity, given that the surfaces have the same roughness and thus area.

### Pluripotent Stem Cells

### Characterization of Pluripotent Stem Cells

Pluripotent stem cells may express one or more of the stage-specific embryonic antigens (SSEA) 3 and 4, and markers detectable using antibodies designated Tra-1-60 and Tra-1-81 (Thomson et al., Science 282:1145 1998). Differentiation of pluripotent stem cells in vitro results in the loss of SSEA-4, Tra-1-60, and Tra-1-81 expression (if present) and increased expression of SSEA-1. Undifferentiated pluripotent stem cells typically have alkaline phosphatase activity, which can be detected by fixing the cells with 4% paraformaldehyde and then developing with Vector Red as a substrate, as described by the manufacturer (Vector Laboratories, Burlingame Calif.). Undifferentiated pluripotent stem cells also typically express Oct-4 and TERT, as detected by RT-PCR.

Another desirable phenotype of propagated pluripotent stem cells is a potential to differentiate into cells of all three germinal layers: endoderm, mesoderm, and ectoderm tissues. Pluripotency of stem cells can be confirmed, for example, by injecting cells into severe combined immunodeficient (SCID) mice, fixing the teratomas that form using 4% paraformaldehyde, and then examining them histologically for evidence of cell types from the three germ layers. Alternatively, pluripotency may be determined by the creation of embryoid bodies and assessing the embryoid bodies for the presence of markers associated with the three germinal layers.

Propagated pluripotent stem cell lines may be karyotyped using a standard G-banding technique and compared to published karyotypes of the corresponding primate species. It is desirable to obtain cells that have a “normal karyotype,” which means that the cells are euploid, wherein all human chromosomes are present and not noticeably altered.

### Sources of Pluripotent Stem Cells

The types of pluripotent stem cells that may be used include established lines of pluripotent cells derived from tissue formed after gestation, including pre-embryonic tissue (such as, for example, a blastocyst), embryonic tissue, or fetal tissue taken any time during gestation, typically but not necessarily before approximately 10-12 weeks gestation. Non-limiting examples are established lines of human embryonic stem cells or human embryonic germ cells, such as, for example the human ES cell lines H1, H7, and H9 (WiCell). Also contemplated is use of the compositions of this disclosure during the initial establishment or stabilization of such cells, in which case the source cells would be primary pluripotent cells taken directly from the source tissues. Also suitable are cells taken from a pluripotent stem cell population already cultured in the absence of feeder cells, as well as a pluripotent stem cell population already cultured in the presence of feeder cells. Also suitable are mutant human ES cell lines, such as, for example, BG01v (BresaGen, Athens, Ga.). Also suitable are cells derived from adult human somatic cells, such as, for examples, cells disclosed in Takahashi et al, Cell 131: 1-12 (2007).

In one embodiment, human embryonic stem cells are prepared as described by Thomson et al. (U.S. Pat. No. 5,843,780; Science 282:1145, 1998; Curr. Top. Dev. Biol. 38:133 ff., 1998; Proc. Natl. Acad. Sci. U.S.A. 92:7844, 1995).

### Culture of Pluripotent Stem Cells

In one embodiment, pluripotent stem cells are cultured on a layer of feeder cells or extracellular matrix protein that support the pluripotent stem cells in various ways, prior to culturing according to the methods of the present invention. For example, pluripotent stem cells are cultured on a feeder cell layer that supports proliferation of pluripotent stem cells without undergoing substantial differentiation. The growth of pluripotent stem cells on a feeder cell layer without differentiation is supported using (i) Obtaining a culture vessel containing a feeder cell layer; and (ii) a medium conditioned by culturing previously with another cell type, or a non-conditioned medium, for example, free of serum or even chemically defined.

In another example, pluripotent stem cells are cultured in a culture system that is essentially free of feeder cells, but nonetheless supports proliferation of pluripotent stem cells without undergoing substantial differentiation. The growth of pluripotent stem cells in feeder-cell free culture without differentiation is supported using (i) an adlayer on a solid substrate surface with one or more extracellular matrix proteins; and (ii) a medium conditioned by culturing previously with another cell type, or a non-conditioned medium, for example, free of serum or even chemically defined.

In an alternate embodiment, pluripotent stem cells are cultured on a surface modified plate containing from at least about 0.5% N, a sum of O and N of greater than or equal to 17.2% and a contact angle of at least about 13.9 degrees in a medium conditioned by culturing previously with another cell type, or a non-conditioned medium, for example, free of serum or even chemically defined.

Culture medium: An example of cell culture medium suitable for use in the present invention may be found in US20020072117. Another example of cell culture medium suitable for use in the present invention may be found in U.S. Pat. No. 6,642,048. Another example of cell culture medium suitable for use in the present invention may be found in WO2005014799. Another example of cell culture medium suitable for use in the present invention may be found in Xu et al (Stem Cells 22: 972-980, 2004). Another example of cell culture medium suitable for use in the present invention may be found in US20070010011. Another example of cell culture medium suitable for use in the present invention may be found in Cheon et al. (BioReprod DOI:10.1095/biolreprod.105.046870; 19 Oct. 2005). Another example of cell culture medium suitable for use in the present invention may be found in Levenstein et al. (Stem Cells 24: 568-574, 2006). Another example of cell culture medium suitable for use in the present invention may be found in US20050148070. Another example of cell culture medium suitable for use in the present invention may be found in US20050233446. Another example of cell culture medium suitable for use in the present invention may be found in U.S. Pat. No. 6,800,480. Another example of cell culture medium suitable for use in the present invention may be found in US20050244962. Another example of cell culture medium suitable for use in the present invention may be found in WO2005065354. Another example of cell culture medium suitable for use in the present invention may be found in WO2005086845.

Suitable culture media may also be made from the following components, such as, for example, Dulbecco's modified Eagle's medium (DMEM), Gibco # 11965-092; Knockout Dulbecco's modified Eagle's medium (KO DMEM), Gibco # 10829-018; Ham's F12/50% DMEM basal medium; 200 mM L-glutamine, Gibco # 15039-027; non-essential amino acid solution, Gibco 11140-050; β-mercaptoethanol, Sigma # M7522; human recombinant basic fibroblast growth factor (bFGF), Gibco # 13256-029.

### Differentiation of Pluripotent Stem Cells

In one embodiment of the present invention, pluripotent stem cells are propagated in culture, while maintaining their pluripotency. Changes in pluripotency of the cells with time can be determined by detecting changes in the levels of expression of markers associated with pluripotency. Alternatively, changes in pluripotency can be monitored by detecting changes in the levels of expression of markers associated with differentiation or markers associated with another cell type.

In an alternate embodiment, pluripotent stem cells are propagated in culture and then treated in a manner that promotes their differentiation into another cell type. The other cell type may be a cell expressing markers characteristic of the definitive endoderm lineage. Alternatively, the cell type may be a cell expressing markers characteristic of the pancreatic endoderm lineage. Alternatively, the cell type may be a cell expressing markers characteristic of the pancreatic endocrine lineage. Alternatively, the cell type may be a cell expressing markers characteristic of the β-cell lineage.

Pluripotent stem cells treated in accordance with the methods of the present invention may be differentiated into a variety of other cell types by any suitable method in the art.

For example, pluripotent stem cells treated in accordance with the methods of the present invention may be differentiated into neural cells, cardiac cells, hepatocytes, and the like.

For example, pluripotent stem cells treated in accordance with the methods of the present invention may be differentiated into neural progenitors and cardiomyocytes according to the methods disclosed in WO2007030870.

In another example, pluripotent stem cells treated in accordance with the methods of the present invention may be differentiated into hepatocytes according to the methods disclosed in U.S. Pat. No. 6,458,589.

For example, pluripotent stem cells may be differentiated into cells expressing markers characteristic of the definitive endoderm lineage according to the methods disclosed in D'Amour et al., Nature Biotechnol. 23:1534-1541, 2005.

For example, pluripotent stem cells may be differentiated into cells expressing markers characteristic of the definitive endoderm lineage according to the methods disclosed in Shinozaki et al., Development 131:1651-1662, 2004.

For example, pluripotent stem cells may be differentiated into cells expressing markers characteristic of the definitive endoderm lineage according to the methods disclosed in McLean et al., Stem Cells 25:29-38, 2007.

For example, pluripotent stem cells may be differentiated into cells expressing markers characteristic of the definitive endoderm lineage according to the methods disclosed in D'Amour et al., Nature Biotechnol. 24:1392-1401, 2006.

Markers characteristic of the definitive endoderm lineage are selected from the group consisting of SOX17, GATA4, Hnf-3beta, GSC, Cerl, Nodal, FGF-8, Brachyury, Mix-like homeobox protein, FGF-4 CD48, eomesodermin (EOMES), DKK4, FGF-17, GATA6, CXCR4, C-Kit, CD99, and OTX2. Suitable for use in the present invention is a cell that expresses at least one of the markers characteristic of the definitive endoderm lineage. In one aspect of the present invention, a cell expressing markers characteristic of the definitive endoderm lineage is a primitive streak precursor cell. In an alternate aspect, a cell expressing markers characteristic of the definitive endoderm lineage is a mesendoderm cell. In an alternate aspect, a cell expressing markers characteristic of the definitive endoderm lineage is a definitive endoderm cell.

For example, pluripotent stem cells may be differentiated into cells expressing markers characteristic of the pancreatic endoderm lineage according to the methods disclosed in D'Amour et al., Nature Biotechnol. 24:1392-1401, 2006.

Markers characteristic of the pancreatic endoderm lineage are selected from the group consisting of Pdx1, HNF-1beta, PTF1a, HNF-6, HB9 and PROX1. Suitable for use in the present invention is a cell that expresses at least one of the markers characteristic of the pancreatic endoderm lineage. In one aspect of the present invention, a cell expressing markers characteristic of the pancreatic endoderm lineage is a pancreatic endoderm cell.

Pluripotent stem cells may be differentiated into cells expressing markers characteristic of the pancreatic endocrine lineage by any method in the art.

For example, pluripotent stem cells may be differentiated into cells expressing markers characteristic of the pancreatic endocrine lineage according to the methods disclosed in D'Amour et al., Nature Biotechnol. 24:1392-1401, 2006.

For example, pluripotent stem cells may be differentiated into cells expressing markers characteristic of the pancreatic endocrine lineage, by the methods disclosed in D'Amour et al., Nature Biotechnol. 24:1392-1401, 2006.

Markers characteristic of the pancreatic endocrine lineage are selected from the group consisting of NGN-3, NeuroD, Islet-1, Pdx-1, NKX6.1, Pax-4, Ngn-3, and PTF-1 alpha. In one embodiment, a pancreatic endocrine cell is capable of expressing at least one of the following hormones: insulin, glucagon, somatostatin, and pancreatic polypeptide. Suitable for use in the present invention is a cell that expresses at least one of the markers characteristic of the pancreatic endocrine lineage. In one aspect of the present invention, a cell expressing markers characteristic of the pancreatic endocrine lineage is a pancreatic endocrine cell. The pancreatic endocrine cell may be a pancreatic hormone-expressing cell. Alternatively, the pancreatic endocrine cell may be a pancreatic hormone-secreting cell.

In one aspect of the present invention, the pancreatic endocrine cell is a cell expressing markers characteristic of the β cell lineage. A cell expressing markers characteristic of the β cell lineage expresses Pdx1 and at least one of the following transcription factors: NGN-3, NRx2.2, NRx6.1, NeuroD, Is1-1, HNF-3 beta, MAFA, Pax4, and Pax6. In one aspect of the present invention, a cell expressing markers characteristic of the β cell lineage is a β cell.

The present invention is further illustrated, but not limited by, the following examples.

### EXAMPLES

### Example 1

### Passage and Maintenance of Human Embryonic Stem Cells as Cell Clusters

The human ES cell lines H1 and H9 were initially maintained on mitomycin C inactivated primary mouse embryonic fibroblasts (MEF). The human embryonic stem cells were switched from MEF feeders to MATRIGEL® (Becton-Dickinson, Bedford, Mass.) over repeated passages.

Treatment of surfaces with MATRIGEL®: Growth Factor Reduced MATRIGEL® was thawed at 4° C. and then diluted 1:30 in cold DMEM/F12 (Invitrogen, Carlsbad, Calif.). Volumes sufficient to cover the surface were added to each 6-cm dish (2 ml) or each well of a 6-well plate (1 ml), and incubated 1 hr at room temp. Treated surfaces were used within a few hours or stored at 4° C. up to two weeks.

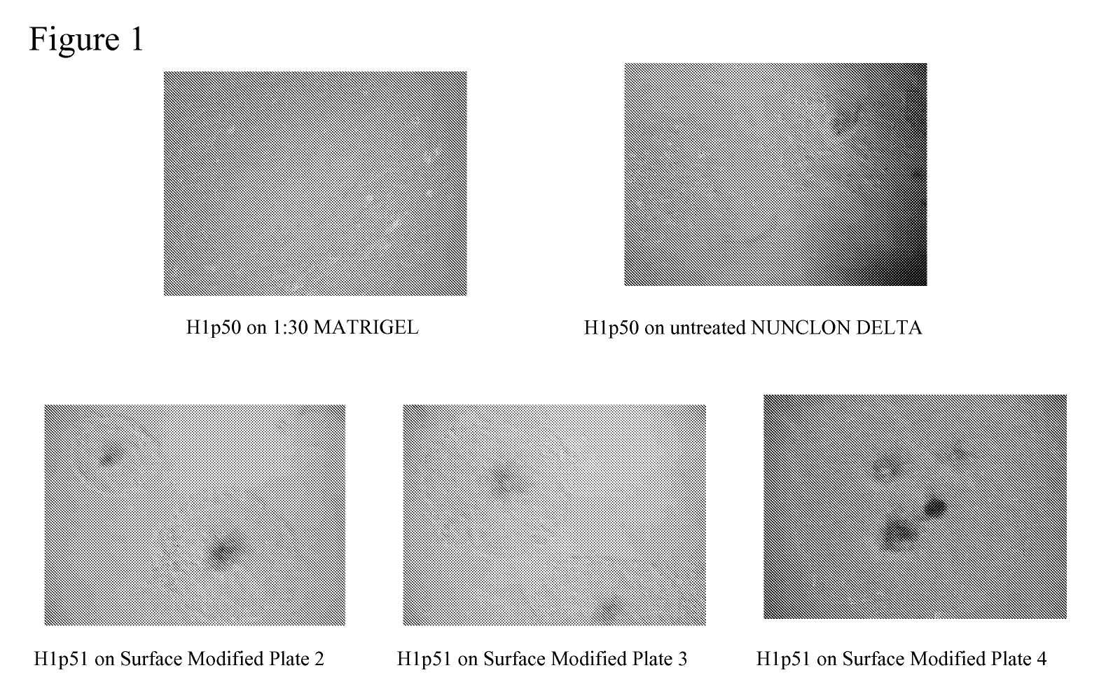

Human embryonic stem cell culture: Undifferentiated human embryonic stem cell colonies (from either the H9 or H1 lines) were harvested from feeder layers by incubation in 1 mg/ml collagenase IV (Sigma-Aldrich, St. Louis, Mo.) in DMEM/F12 for 10 minutes, followed by scraping with a pipette. Cell clumps were pelleted by centrifugation at 600×g for four minutes and the pellet dispersed gently with a 2-ml pipette to break colonies into small clusters of cells. These cell clusters were seeded onto MATRIGEL®-treated dishes in media conditioned with mouse embryonic fibroblasts (MEF-CM), further supplemented with bFGF (8 ng/ml; R&D Systems, Minneapolis, Minn.), at 50-150 colonies per 6-cm dish in 5 ml growth medium. Medium was changed daily. Colonies on MATRIGEL® in MEF-CM became large and were passed when they occupied 70-80% of the surface area, approximately every 3-4 days. The human embryonic stem cells in the colonies had a high nucleus to cytoplasm ratio and had prominent nucleoli, similar to human embryonic stem cells maintained on feeders (FIG. 1). Differentiated cells represented less than 5% of total cells in culture.

For routine passage of cells in MEF-CM on MATRIGEL®, cells were incubated in 1 mg/ml collagenase IV in DMEM/F12 for up to 60 minutes and removed from the dishes by forceful streams of DMEM/F12 with scraping. Cells were pelleted, dispersed, and seeded at a 1:3 or 1:4 ratio.

### Example 2

### Passage of Human Embryonic Stem Cells as Single Cells

Human embryonic stem cells of the cell line H9 were grown as single cells according to the methods disclosed in U.S. patent application Ser. No. 12/164,795, assigned to LifeScan Inc. Cells were passaged by treatment with TrypLE™ Express for five minutes at 37° C., and seeded at 10,000 cells/cm² substrate surface.

### Example 3

### Attachment, Cultivation and Maintenance of Pluripotency of Human Embryonic Stem Cells Using Surface Modified Plates Lacking Extracellular Matrix Protein/Components and Feeder Cells

Human embryonic stem cells of the line H1, at passage 49 were maintained in MEF conditioned media on NUNCLON DELTA™ plates treated with a 1:30 dilution of growth factor reduced MATRIGEL®, prior to study. Cells were dissociated from the surface for passage by 1 mg/ml collagenase dissociation or by manual scraping.

These cells were then seeded onto two untreated wells of the surface modified plates (6-well format). Additionally, one well of each plate was treated with 0.1% xeno-free human gelatin as a control. Cells were also plated directly onto untreated and gelatin-treated wells of COSTAR™ (cat. no. 3516; Corning, Corning, N.Y.), FALCON™ (cat. no. 351146; Becton Dickinson, Franklin Lakes, N.J.) and NUNCLON DELTA™ (cat. no. 140675; Thermo Fisher Scientific, Roskilde, Denmark) 6-well plates for negative controls, and plated onto wells treated with 1:30 dilution of growth factor reduced MATRIGEL® to provide as positive controls. In all treatments cells were maintained in MEF conditioned media.

We observed that after two passages, surface modified plates 2, 3, and 4 had attached embryonic stem cell colonies, which re-attached to the plates and grew following enzymatic dissociation. There was no apparent difference in rate of attachment or growth in gelatin or untreated wells from surface modified plates 2, 3, or 4.

Cells mechanically dissociated from plates treated with 1:30 dilution of growth factor reduced MATRIGEL® were poorly attached to surface modified plates 2, 3, and 4, while cells enzymatically dissociated with 1 mg/ml collagenase were well attached in gelatin or untreated wells from surface modified plates 2, 3, or 4.

H1p49 embryonic stem cells added to surface modified plates 1 and 5-12 and to untreated or gelatin treated NUNCLON DELTA™ plates, FALCON™ plates, and COSTAR™ plates did not attach. The same cells did attach to plates treated with 1:30 dilution of growth factor reduced MATRIGEL®, indicating that the cells were competent to attach to a substrate surface.

Normal passage time for embryonic stem cells of the H1 line plated on 1:30 dilution of growth factor reduced MATRIGEL® was 3-4 days, however cells plated on surface modified plates 2, 3 and 4 took 7 days of culturing before they were ready for passage. This was probably due to the reduced rate of attachment on the treated surfaces, since more starting colonies were apparent on MATRIGEL™-treated surfaces immediately after plating than on Surfaces 2, 3 and 4.

The passage (p) 50 Cells were split at a 1 to 2 ratio and half of the sample was collected for RNA purification and tested for expression of pluripotency markers (Table 1). The other half of each sample was replated to surface modified plates. Colonies that formed at this passage (p51) also required 7 days of culturing before they were ready to be passaged, and the small colonies that developed after only 4 days of culturing are shown in FIG. 1. These colonies maintained classical embryonic stem cell colony morphology.

Cultures were stopped at passage 4 on surface modified plates 2, 3 and 4 and samples were assayed for pluripotency markers by qRT-PCR (Table 2) and differentiated to a definitive endoderm fate (DE). Cells at passage 4 maintained expression of the classical pluripotency markers: OCT4, NANOG, SOX2, and TERT. Furthermore, the cells were able to differentiate to a definitive endoderm fate upon exposure to a media containing DMEM/F12, 100 ng/ml Activin A, 20 ng/ml Wnt3a, and 0.5-2.0% FBS (Table 3) indicating that pluripotency was maintained in the cells through passage 4.

### Example 4

### Attachment, Cultivation and Maintenance of Pluripotency of Human Embryonic Stem Cells on Surface Modified Plates Lacking Extracellular Matrix Protein/Components and Feeder Cells: Effects of Rho Inhibition and Rho Kinase Inhibition

Human embryonic stem cells of the line H1, at passage 49 were maintained in MEF conditioned media on NUNCLON DELTA™ plates treated with a 1:30 dilution of growth factor reduced MATRIGEL®, prior to study. Cells were dissociated from the surface for passage by 1 mg/ml collagenase dissociation.

These cells were then seeded onto untreated wells of surface modified plates (6-well format). Cells were also plated directly onto untreated and gelatin-treated wells of COSTAR™, FALCON™, and NUNCLON DELTA™ 6-well plates for negative controls and plated onto wells treated with 1:30 dilution of growth factor reduced MATRIGEL® to provide as positive controls. In all treatments cells were maintained in MEF conditioned media.

Human embryonic stem cells of the line H1, at passage 49 added to surface modified plates 1 and 5-12 and to untreated or gelatin treated NUNCLON DELTA™ plates and COSTAR™ plates did not attach, however, they did attach to surface modified plates 2, 3, and 4. The same cells did attach to plates treated with 1:30 dilution of growth factor reduced MATRIGEL®, indicating that the cells were competent to attach to a substrate surface.

Normal passage time for H1 embryonic stem cells plated on 1:30 dilution of growth factor reduced MATRIGEL® was 3-4 days, however cells plated on surface modified plates 2, 3 and 4 took 7 days of culturing before they were ready for passage. This was probably due to the reduced rate of attachment on the surface modified plates, since more starting colonies were apparent on MATRIGEL®-treated surfaces immediately after plating than on surface modified plates 2, 3 and 4.

The passage (p) 50 Cells were split at a 1 to 2 ratio and half of the sample was collected for RNA purification and tested for expression of pluripotency markers (Table 1). The other half of each sample was replated to surface modified plates. Colonies that formed at this passage (p51) also required 7 days of culturing before they were ready to be passaged, and the small colonies that developed after only 4 days of culturing are shown in FIG. 1. These colonies maintained classical embryonic stem cell colony morphology.

Due to the delay in passage, the cells were split at a 1 to 2 ratio and half of the passage 4 samples were plated in MEF conditioned media or MEF conditioned media supplemented with the Rho kinase (ROCK) inhibitor, Y-27632, at a 10 μM concentration in an attempt to improve cell growth kinetics. Cells were kept in the plating media for 48 hours after passage at which time the media was changed to fresh unsupplemented MEF conditioned media.

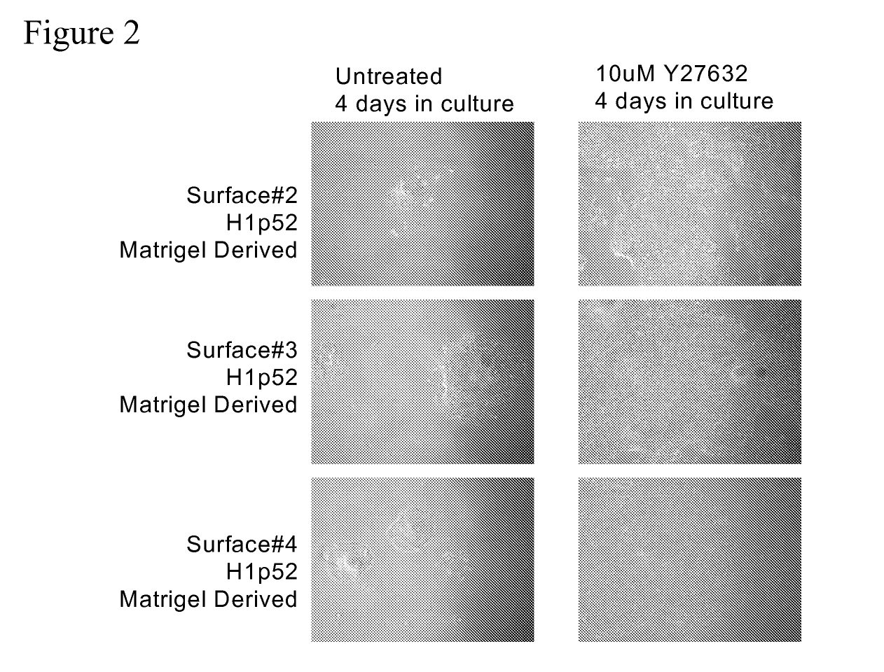

The addition of Y-27632 at a 10 μM concentration significantly increased plating efficiency of the cells (p52) and the improvement in colony growth was apparent after 4 days post-plating (FIG. 2). Alternatively, prior to collagenase dissociation, human embryonic stem cells of the line H1 were also treated with 0.5 ng/ml of a cell permeable form of the Rho inhibitor, C3 exotransferase, which also increased the plating efficiency of the cells.

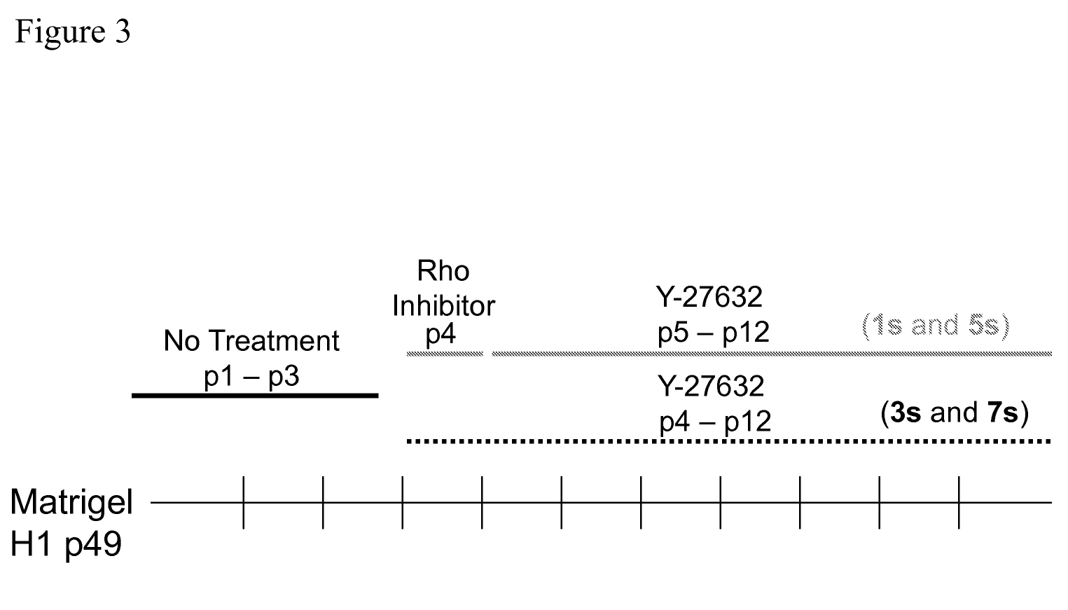

While cells plated in 10 μM Y-27632 could be passaged 4 days after plating, cells plated without the ROCK inhibitor were not ready to be split 4 days after plating. Cells treated with Rho inhibitor, C3 exotransferase were also not ready for passage 4 days after plating and cells exhibited increased differentiation to a fibroblast-like morphology. Consequently, cells treated with Rho inhibitor at passage 4 were treated with Y-27632 at all subsequent passages (FIG. 3).

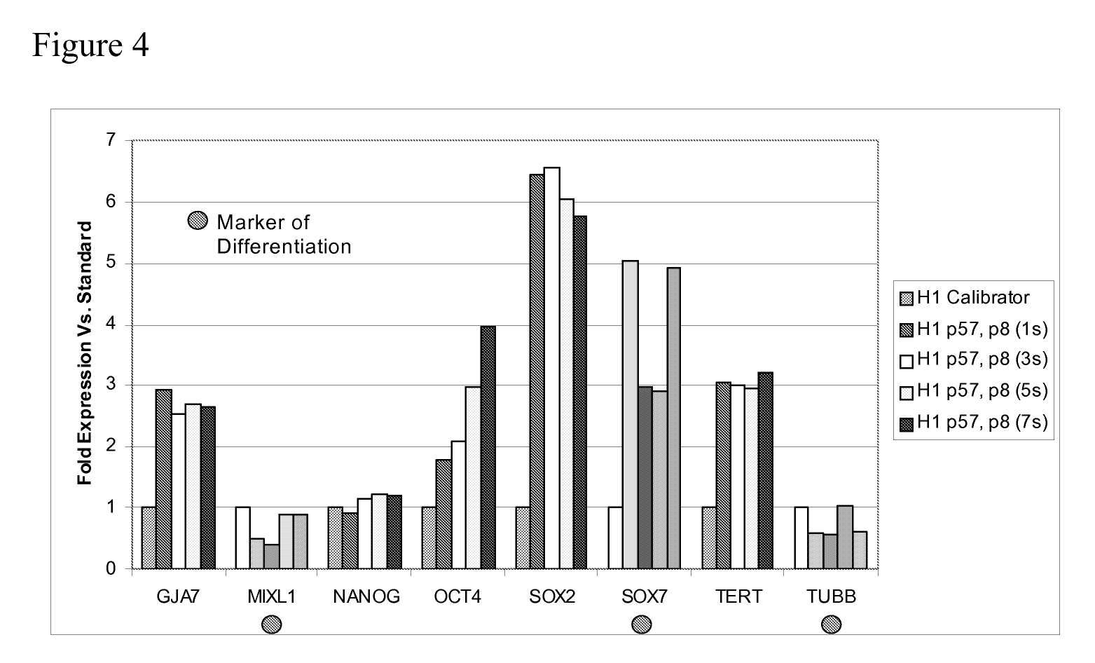

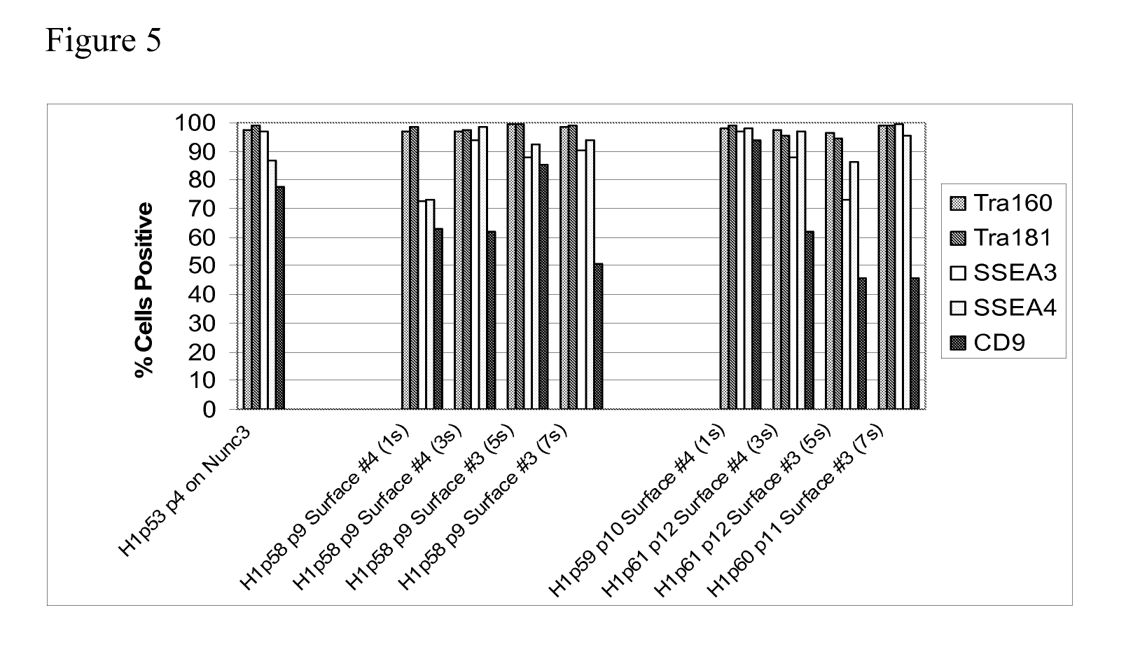

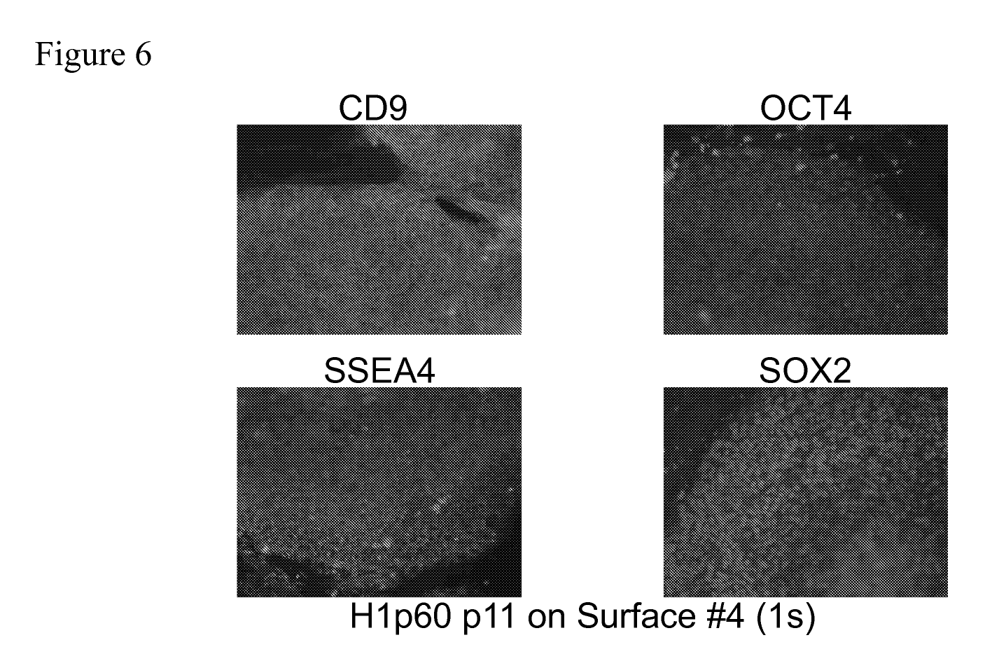

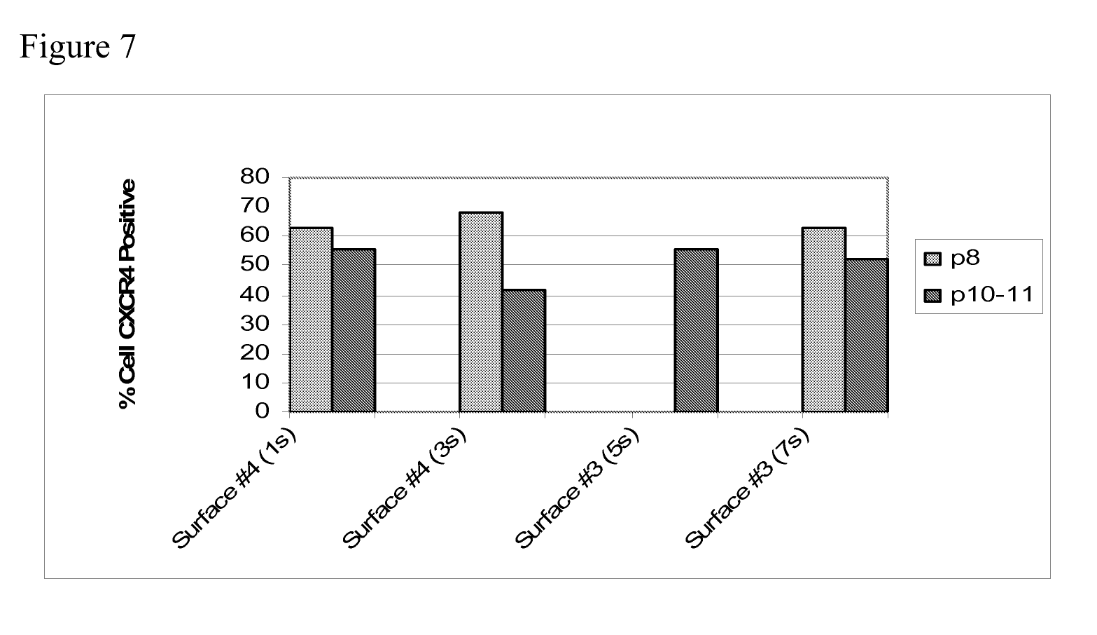

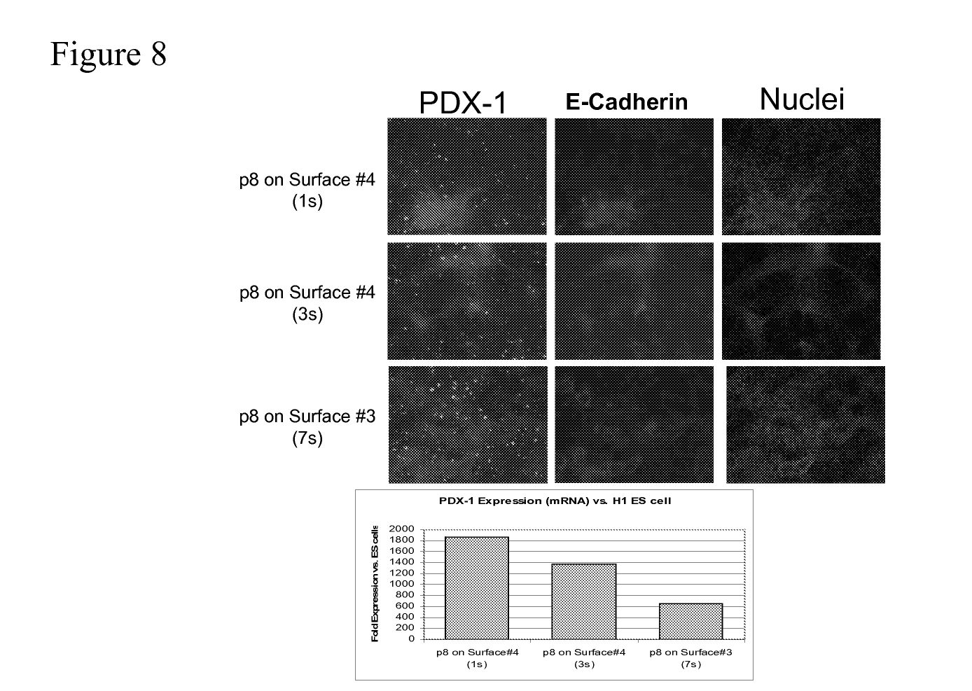

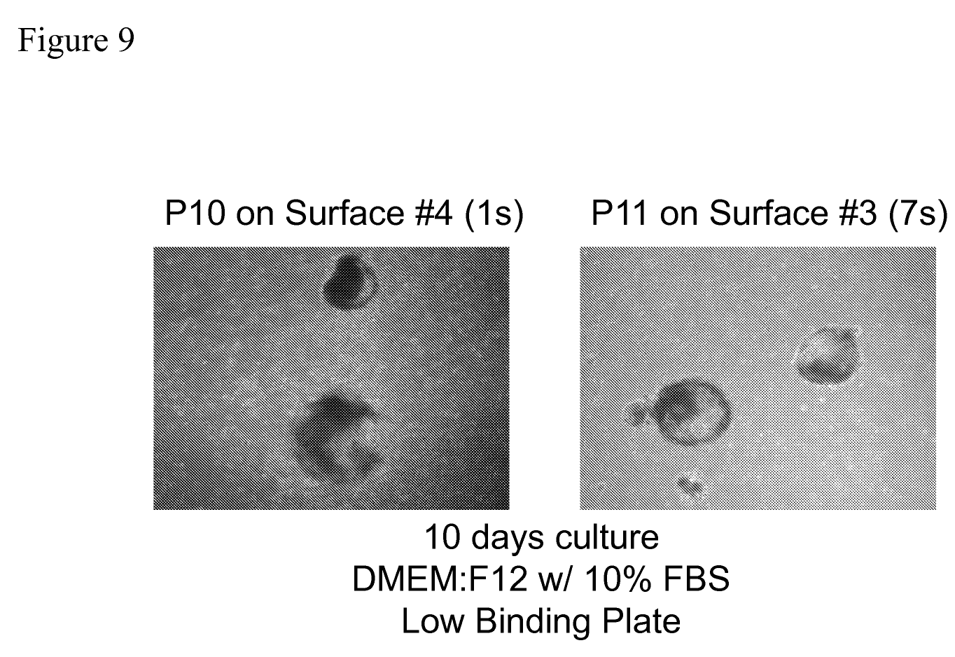

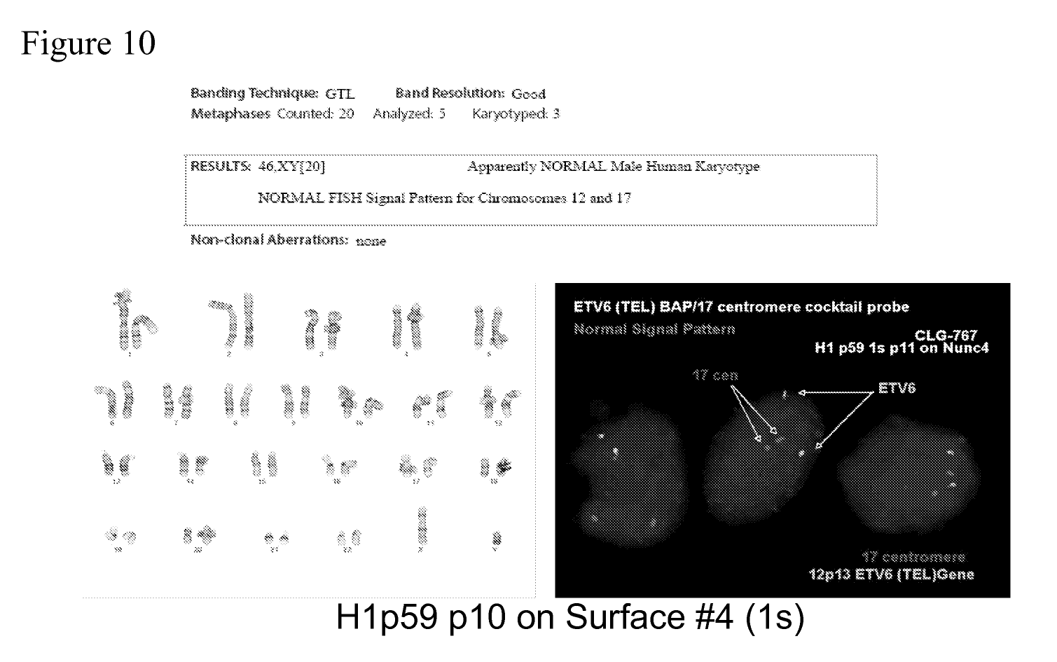

Cells were further passaged to at least 10 passages on surface modified plates 3 and 4 and were tested for the presence of markers associated with pluripotency: genes by qRT-PCR; cell surface marker expression by flow cytometry; and immunofluorescence of cell surface and nuclear proteins (FIGS. 4-6). Cellular pluripotency was also confirmed by testing their capacity to differentiate to definitive endoderm, pancreatic endoderm, and form embyroid bodies composed of the three germ layers (FIGS. 7-9). Cells were also tested for karyotypic stability, and we observed that cells could maintain a normal karyotype (FIG. 10).

### Example 5

### Attachment and Detachment of Human Embryonic Stem Cells Through Rho Kinase Inhibition

Human embryonic stem cells of the line H9, at passage 40 were maintained in MEF conditioned media on Nunclon Delta™ plates treated with a 1:30 dilution of growth factor reduced MATRIGEL® prior to study. Cells were dissociated from the surface for passage by 1 mg/ml collagenase dissociation or by manual scraping.

These cells were then seeded onto either surface modified plates 2, 3, 4 or 13 (12-well format) in the presence of increasing amounts one of the following Rho Kinase inhibitors: Y-27632 (from Sigma, St. Louis, Mo. or EMD, San Diego, Calif.), Fasudil (Sigma), or Hydroxyfasudil (Sigma), and maintained for 3 days, each day replacing the media and compound. At the end of day three, media was removed and the plates were stained with Crystal Violet (0.5% in water) to visualize colonies.

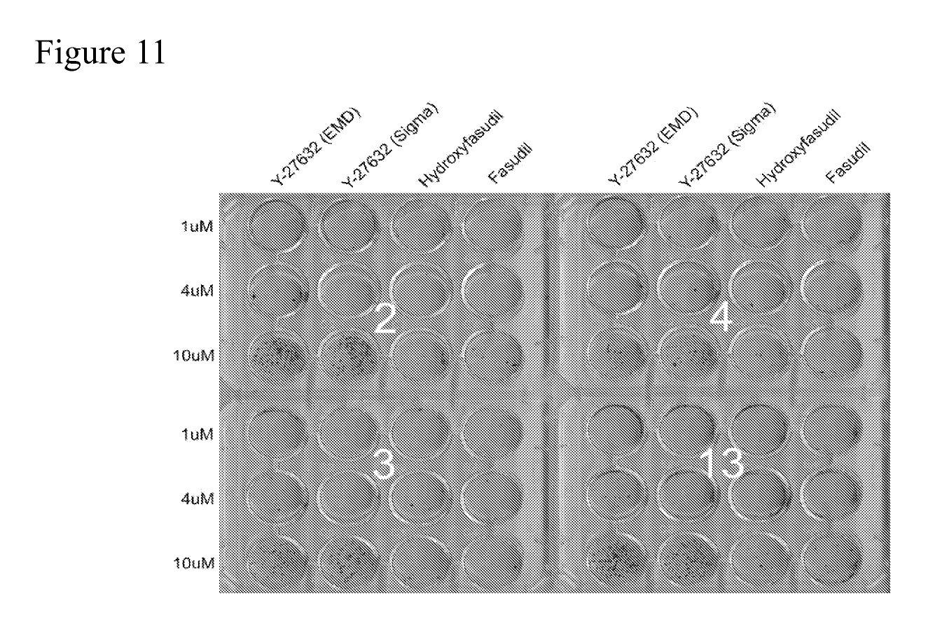

We observed that by day three, surface modified plates 2, 3, 4 and 13 had attached embryonic stem cell colonies in the presence of increasing amounts of Rho kinase inhibitor. Best results were obtained through the use of Y-27632 (10 μM), although some colonies could be observed to attach and grow with the Rho kinase inhibitors, Fasudil and Hydroxyfasudil (FIG. 11).

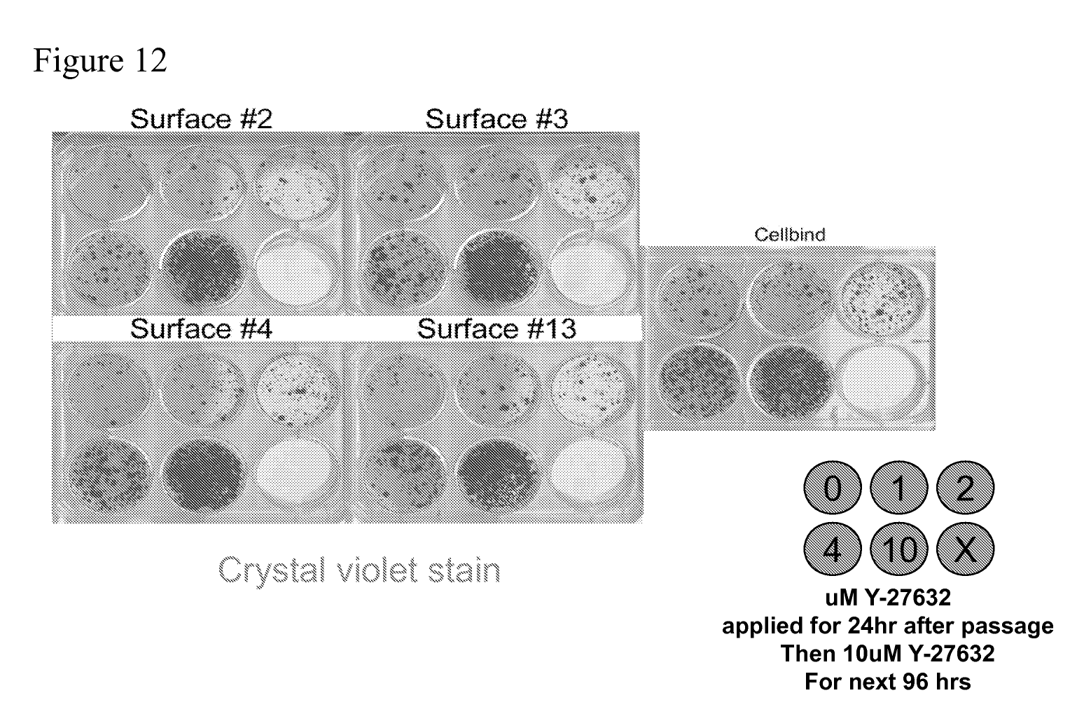

We attempted to determine the optimal dose of Y-27632 to promote cell binding, by treating cells with a range of plating concentrations of Y-27632 for the first day of culture. After the first day in culture cells were treated on subsequent days with a 10 μM concentration of Y-27632. We observed that the maximal concentration to stimulate attachment and growth of embryonic stem cells was 10 μM (FIG. 12) and that this occurred on surface modified plates 2, 3, 4, 13 and CELLBIND™ (Corning, Corning, N.Y.).

The effect of treating the cells continuously with a single dose of Y-27632 on attachment and growth was also tested. The cells were dosed with 0, 1, 4, or 10 μM Y-27632 for 4 days. Some binding was observed on surface modified plates without treatment (0 μM), however the optimal concentration to stimulate attachment and growth of ES cells was 10 μM Y-27632 (Table 4) on surface modified plates 2, 3, 4, 13.

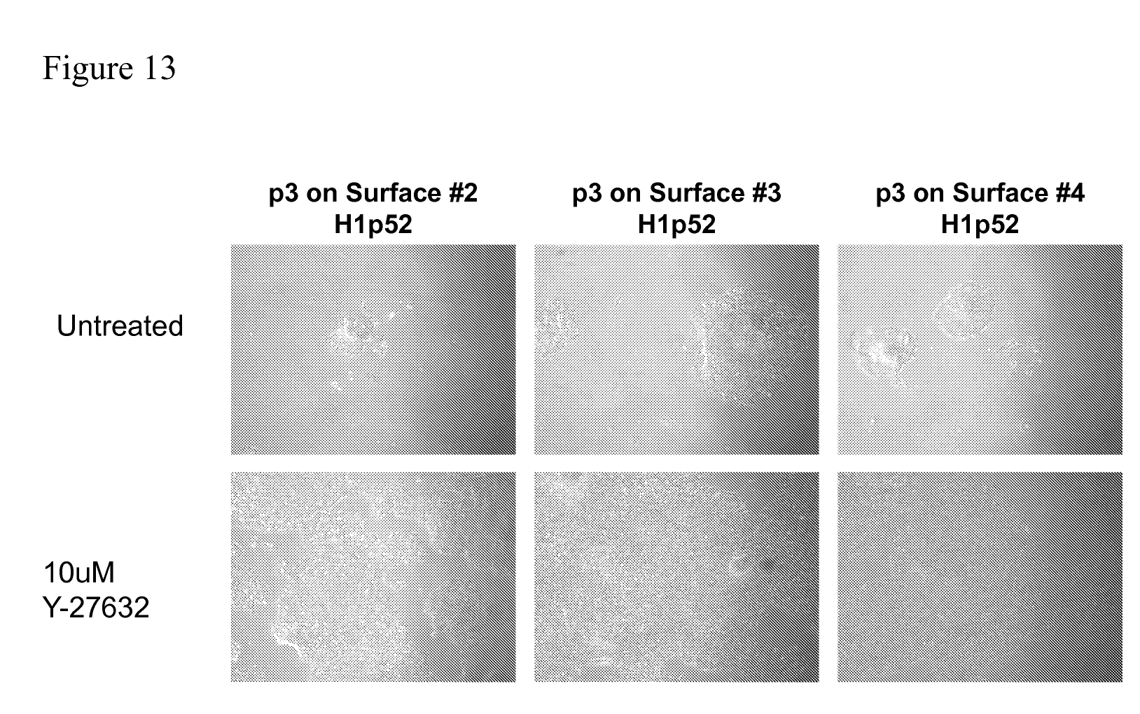

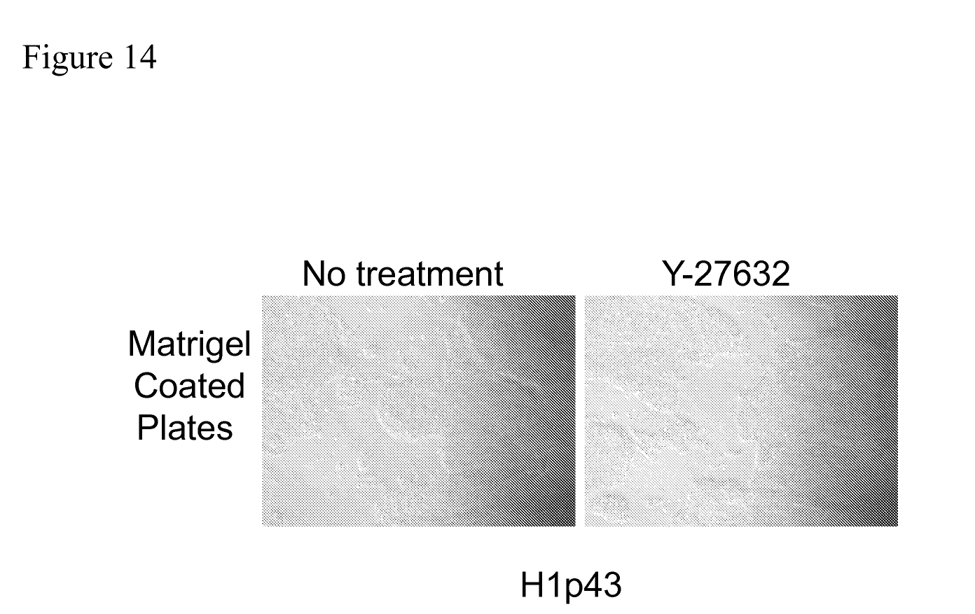

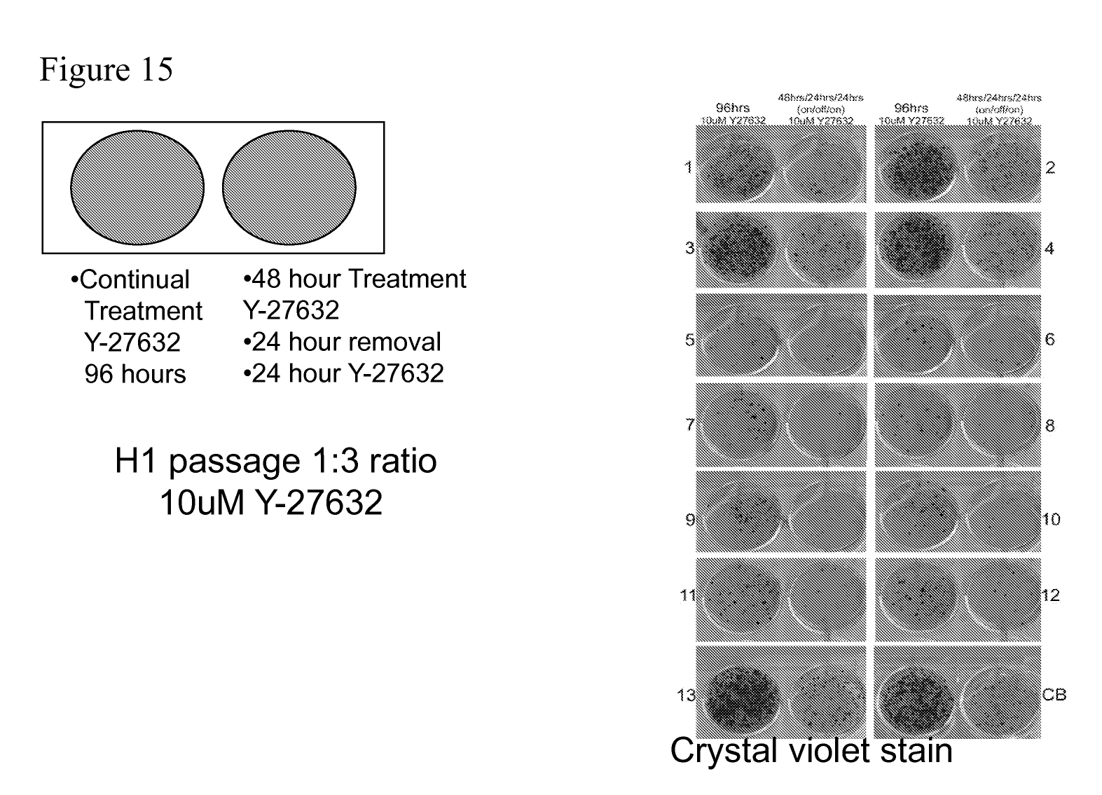

Since the addition of ROCK inhibitor significantly enhances the plating and growth kinetics on surface modified plates 2, 3, and 4 versus untreated cells (FIG. 13), we wished to determine if this was due to maintenance of proper cell attachment or due to increased cell proliferation. We observed that Rho Kinase inhibition does not increase cell proliferation, because cells treated with Y-27632 grow at a similar density as untreated cells (FIG. 14). Instead, Y-27632 treatment maintains the attachment of cells to the surface and allows them to grow with normal proliferation kinetics (FIG. 15). Removal of a Rho Kinase inhibitor from the growth media of embryonic stem cells plated in the presence of Rho kinase inhibitor results in detachment of the cells from the surface. The formation of embryoid bodies with differentiation to the 3 germ lineages is accomplished by culturing embryonic stem cells in a suspension. Consequently, although later reapplication of a Rho kinase inhibitor restored attachment of cells (FIG. 15), as expected, substantial differentiation of the embryonic stem cell culture was observed in samples where Rho kinase inhibitor was withdrawn for 24 hours of culture and cells were allowed to detach, grow in suspension for 24 hours and Rho kinase inhibitor was then reapplied.

### Example 6

### H9 Human Embryonic Stem Cells passaged with TrypLE™ Express on Surface Modified Plates Show Improved Adhesion with Y-27632

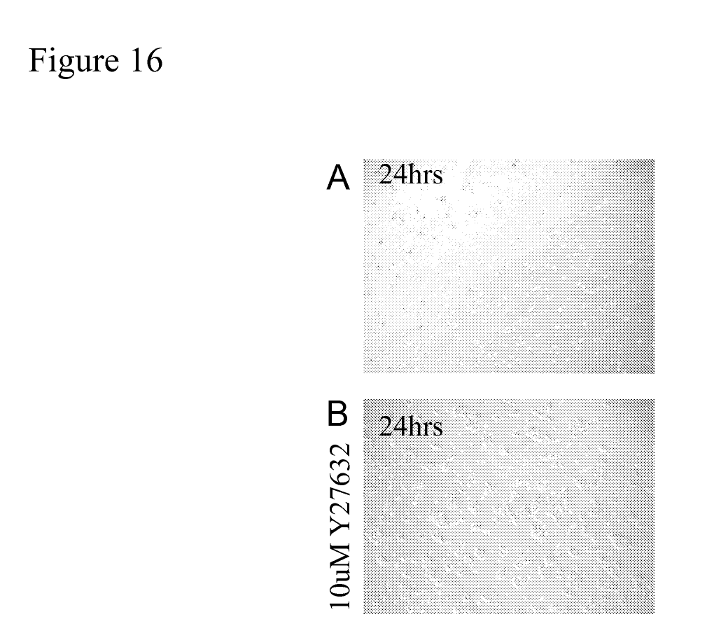

Initial passaging of H9 human embryonic stem cells onto surface modified plates. Adhesion is improved with continuous 10 μM of Y-27632. This is true for the four surface modified plates tested: 2, 3, 4 and 13. Images of H9 cells 24 hours after seeding on Surface 3 are shown in FIG. 16.

### Example 7

### H9 single Human Embryonic Stem Cells Passaged with TrypLE™ Express on Surface Modified Plates Remain Pluripotent

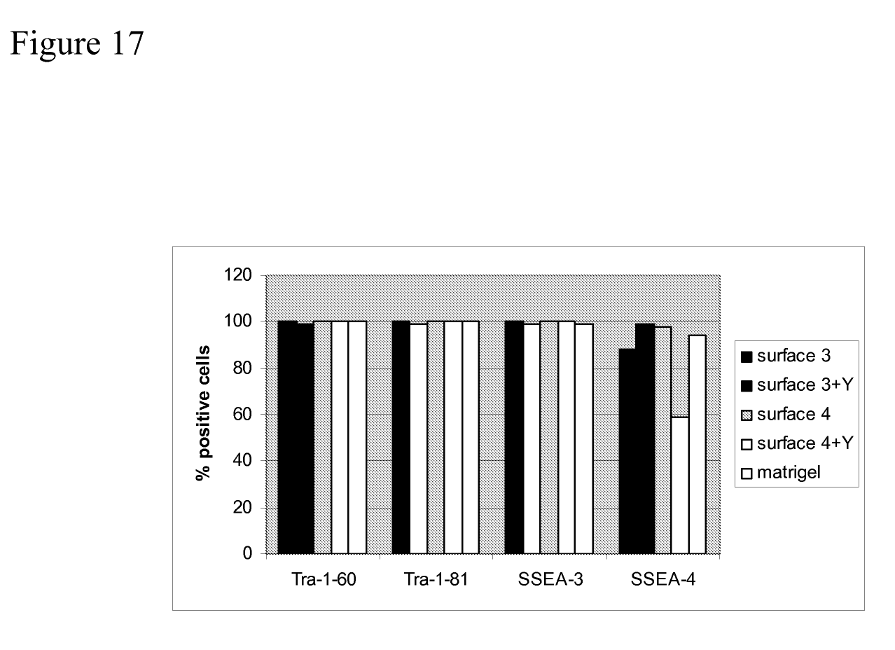

Human embryonic stem cells are pluripotent and have the ability to differentiate into all cell lineages. The pluripotent state of the cells must be maintained by the surface on which they grow. To determine if the surface modified plates can maintain human embryonic stem cell pluripotency, the human embryonic stem cells were passaged 38 times with collagenase and 38 times with TrypLE™ Express followed by 5 passages on surface modified plate 3 (Surface 3), surface modified plate 4 (Surface 4) or MATRIGEL® at 1:30 dilution. 10 μM of Y-27632 was added to the media of indicated samples. The expression, of pluripotency markers Tra-1-60, Tra-1-81, SSEA-3 and SSEA-4, was evaluated by flow cytometry. Results are shown in FIG. 17. The percentage of positive cells is indicated on the y-axis. Single human embryonic stem cells grown on surface modified plates 3 and 4 can maintain their pluripotency.

### Example 8

### Rho Kinase Inhibition Promotes Adhesion and growth of Cells from the Human Embryonic Stem Cell line H9, Grown as Single Cells on Surface Modified Plates upon Transfer from MATRIGEL®

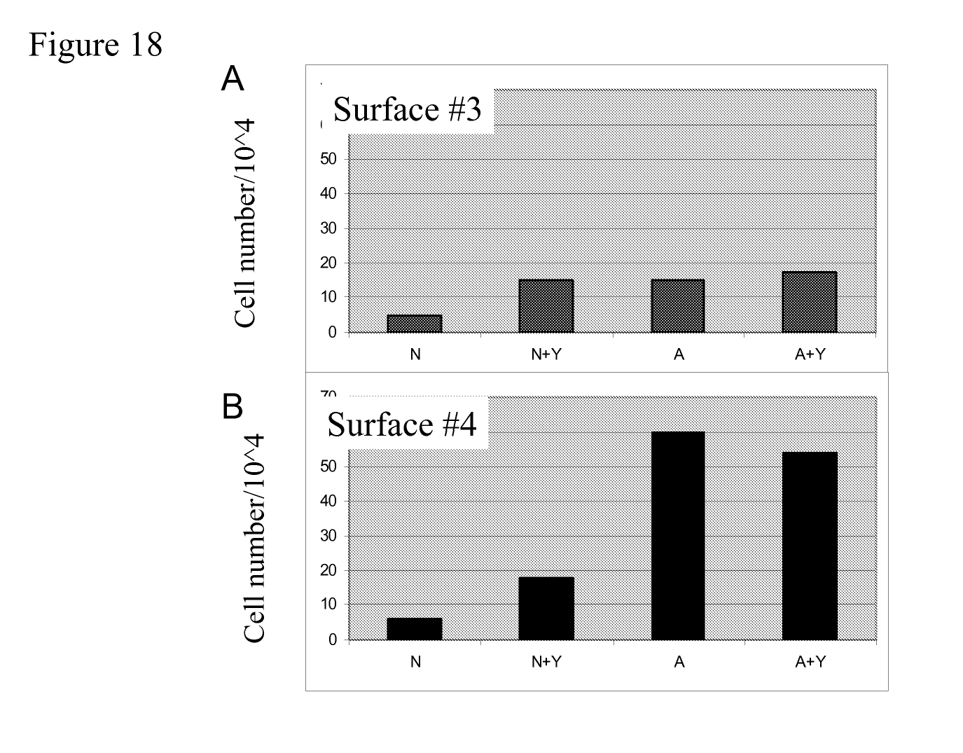

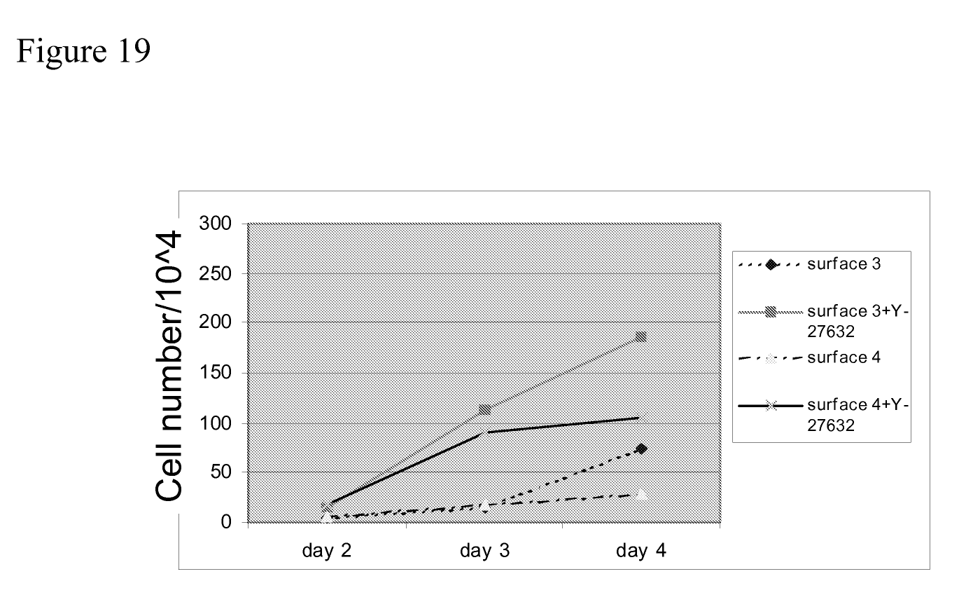

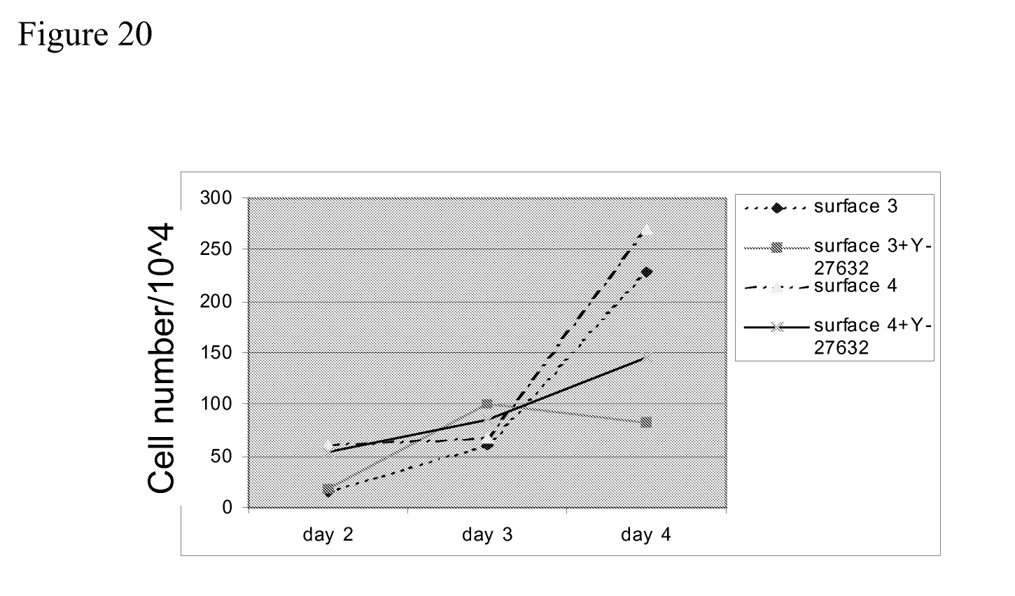

The role of Y-27632 in human embryonic stem cell adhesion and cell growth was studied in relation to the surface modified plates. H9 human embryonic stem cells were passaged 38 times with collagenase and 50 times with TrypLE™ Express followed by seeding onto Surfaces modified plates 3 or 4 (naïve cells). Alternatively H9 human embryonic stem cells passaged 38 times with collagenase and 38 times with Triple Express followed by 9 passages on surface modified plate 3 (Surface 3, acclimated cells), or surface modified plate 4 (Surface 4, acclimated cells). Cells were seeded at a density of 10⁴ cells/cm² in MEF conditioned media and grown for two days with or without the presence of 10 μM of Y-27632. Results are shown in FIG. 18. Y-27632 improves attachment of naïve cells to surface modified plates 3 and 4. Y-27632 did not improve attachment of acclimated cells to surface modified plates 3 or 4. Surface modified plate 3 improved attachment and/or growth of naïve cells. Surface modified plate 4 improved attachment and/or growth of acclimated human embryonic stem cells. The cells were followed for a total of 4 days (FIGS. 19 and 20). The naïve single cells exhibited an increase growth rate when cultured with 10 μM Y-27632 with surface modified plate 3 showing a slight advantage (FIG. 19). The acclimated single cells exhibited improved growth rates without the 10 μM of Y-27632 (FIG. 20).

### Example 9

### Surface Modified Plates can be used to Screen Compounds

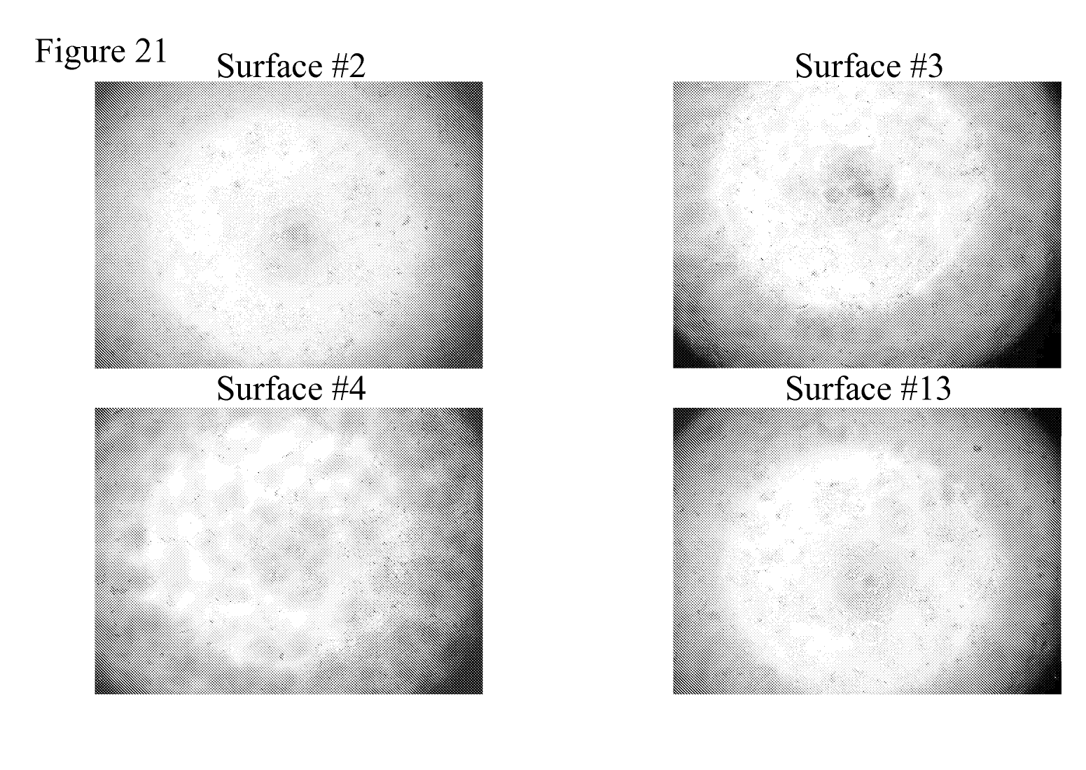

Surface modified plates in 96-well configuration and in the Society for Biomolecular Screening (SBS) standard format can be used for growing single human embryonic stem cells in the presence of 10 μM Y-27632. Images of H9 single cells plated in 96-well plate wells are shown in FIG. 21. This would allow for the screening of compounds directly in 96-well plates with no interfering cells or adlayers, such as mouse embryonic fibroblasts or MATRIGEL®, respectively.

### Example 10

### Single Embryonic Stem Cells Cultured on Surface Modified Plates are able to Differentiate into Definitive Endoderm

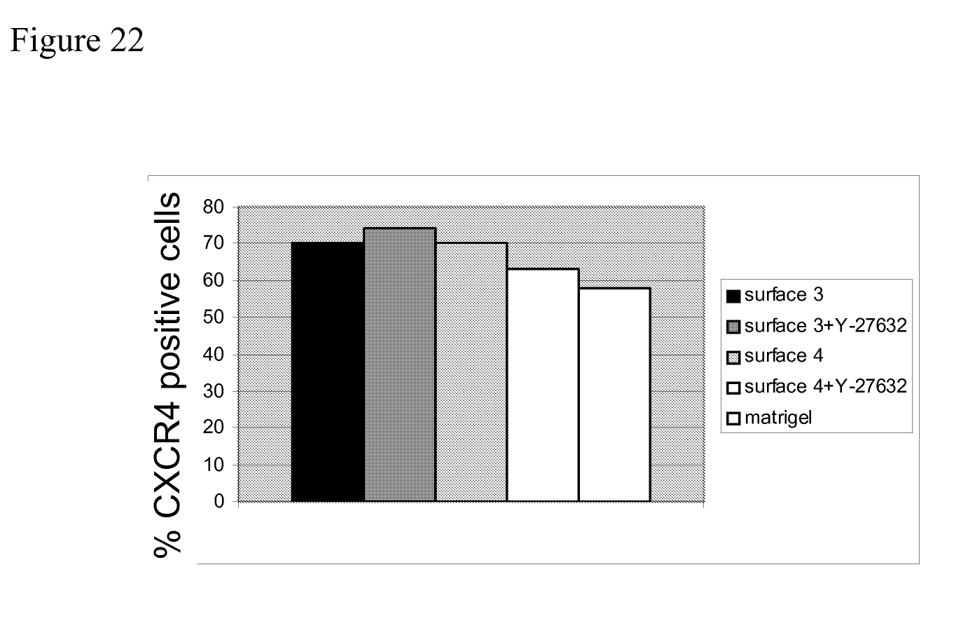

One goal is to differentiate human embryonic stem cells into different cell lineages. To determine if surface modified plates can support differentiation, human embryonic stem cells were passaged 38 times with collagenase and 38 times with TrypLE™ Express followed by 9 passages on surface modified plate 3 (Surface 3), or surface modified plate 4 (Surface 4). As a positive control, human embryonic stem cells were grown on MATRIGEL® at 1:30 dilution. 10 μM of Y-27632 was added to the media during expansion of indicated cell samples. After cell expansion, the ability of the cultured cells to form definitive endoderm was evaluated. Briefly, 70% confluent cultures were treated with 100 ng/ml Activin A, 10 ng/ml Wnt3a and 0.5% FBS in DMEM-F12 media for two days. The treatment was followed by 3 days with 100 ng/ml Activin A and 2% FBS in DMEM/F12. Cells differentiated into definitive endoderm are identified by CXCR4 protein expression, via flow cytometry (FIG. 22). The percentage of positive cells is indicated on the y-axis. Human embryonic stem cells cultured as single cells can differentiate into definitive endoderm in the presence or absence of Y-27632 on surface modified plates 3 and 4.

### Example 11

### Single Embryonic Stem Cells Cultured on Surface Modified Plates are able to Differentiate into Pancreatic Endoderm

After completion of the definitive endoderm protocol, the cells were incubated for 3 days with FGF-7 (50 ng/ml; R&D Systems), the sonic hedgehog inhibitor, KAAD cyclopamine (2.5 μM; Sigma-Aldrich) and 2% FBS in DMEM-F12 medium. At this point, cells not treated with Y-27632 during expansion detached from the surface modified plates 3 and 4. The cells treated with Y-27632 during expansion, were incubated an additional four days with FGF-7 (50 ng/ml), KAAD cyclopamine (2.5 μM), Retinoic Acid (1 μM; Sigma-Aldrich) and 1% B27 (Invitrogen) in DMEM-F12 (posterior foregut stage, PF). After this time, cells were incubated an additional four days in Exendin 4 (50 ng/ml; Sigma-Aldrich), DAPT (1 μM; Calbiochem), and 1% B27 in DMEM-F12. Differentiation was continued to the pancreatic endoderm stage (EN). This entailed a three-day treatment with CMRL medium (Invitrogen) containing 50 ng/ml, HGF, IGF (R&D Systems), and Exendin 4 (50 ng/ml), and 1% B27. RNA samples were taken at stages PF and EN from one well of the surface modified plates 3 and 4. These samples were then analyzed by real-time PCR at this step for pancreatic markers PDX1, NKX6.1, NKX2.2, PAX4, NEUROD, HNF3-beta, PTF1-alpha, Insulin and AFP. Evaluation of the same pancreatic endoderm markers was repeated at this stage. RNA samples from untreated human embryonic stem cells of the same line were subjected to real-time PCR in parallel to treated samples. Treated samples were normalized to untreated controls set to a fold change of 1. PDX1 and insulin expression was monitored and compared between surface modified plates.

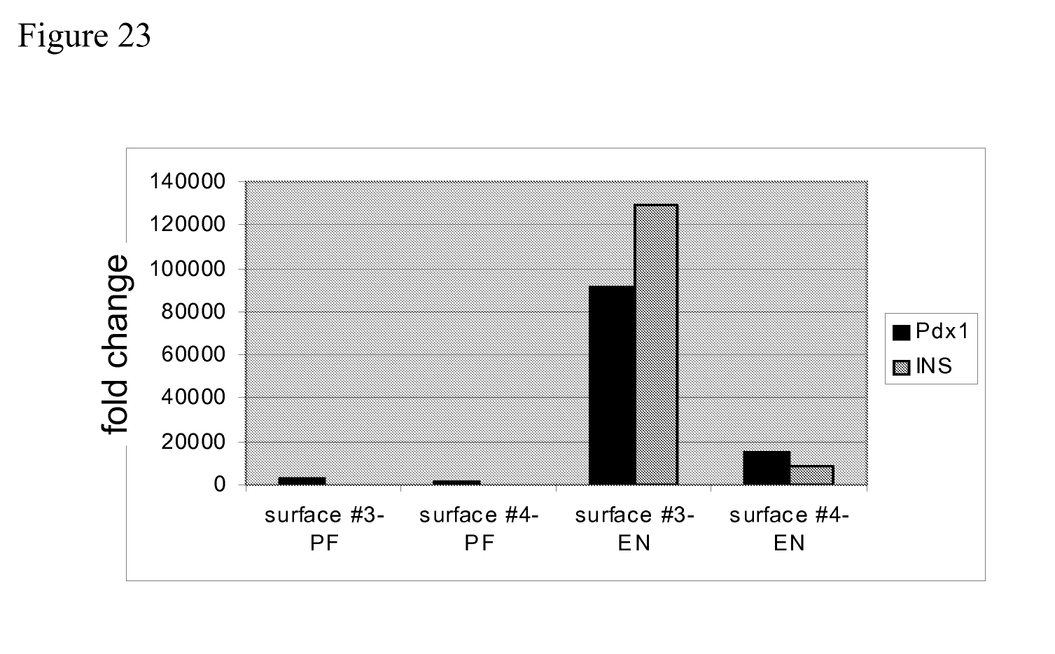

Induction of pancreatic endoderm markers was observed from cells treated on surface modified plates 3 and 4, although expression was higher with cells treated on surface modified plate 3 (FIG. 23). Both surface modified plates in the presence of Y-27632 during expansion can support the differentiation of single human embryonic stem cells to posterior foregut and pancreatic endoderm whereas single cells not treated with Y-27632 during expansion detached prior to posterior foregut differentiation.

### Example 12

### H1 and H9 Human embryonic stem cells Adhere to Surface Modified Plates and the Adherence is Enhanced by Treating Cells with Y-27632

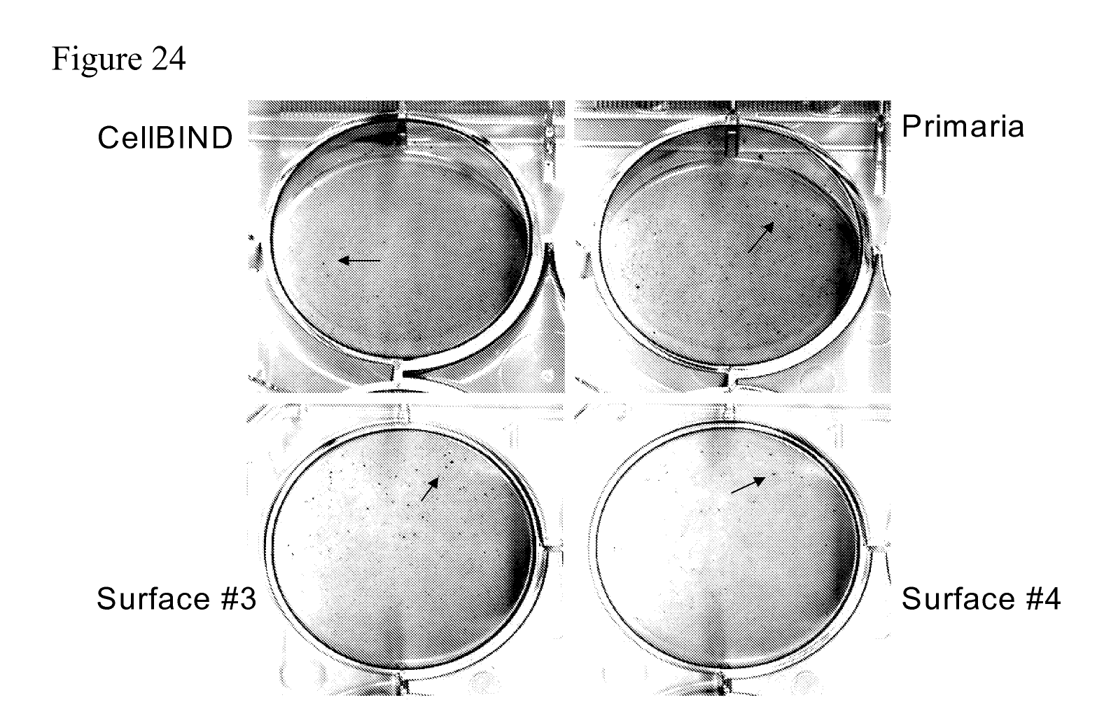

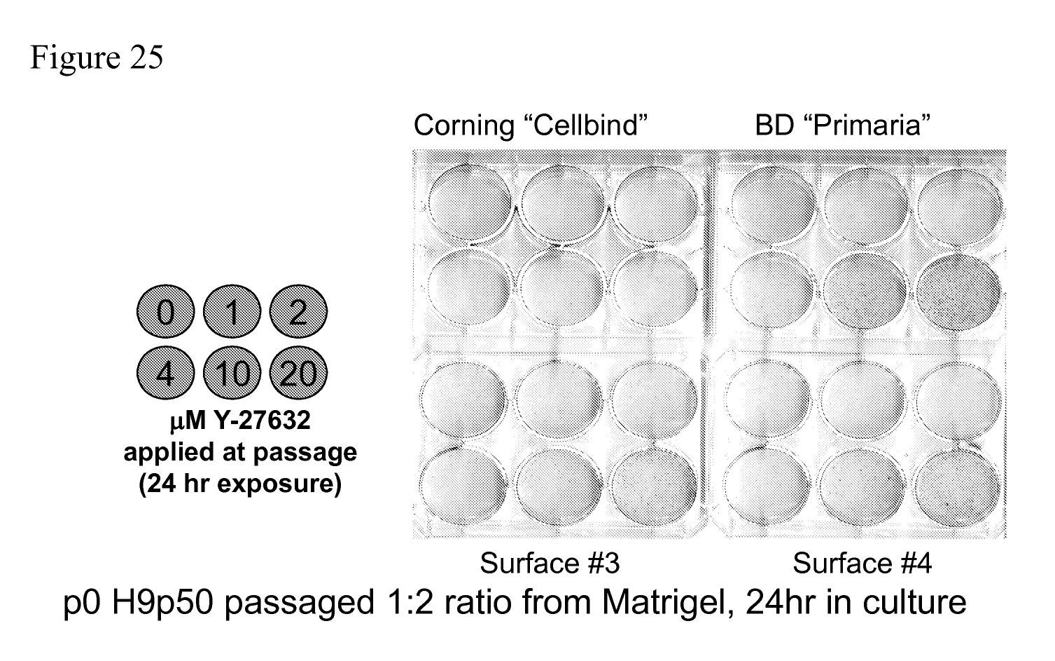

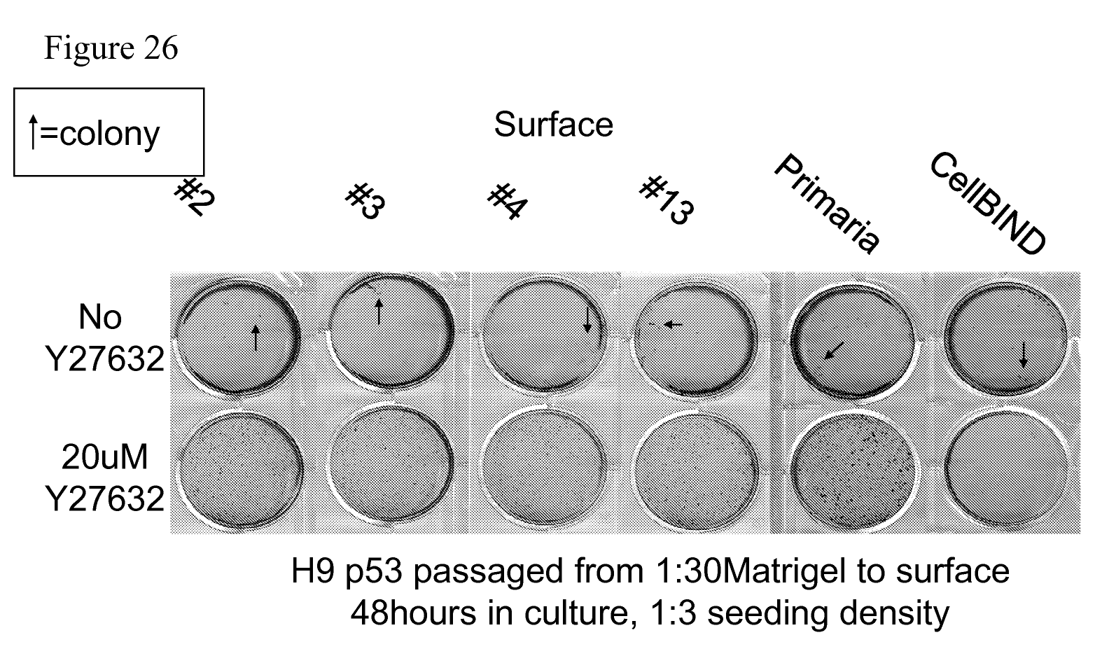

Passage 49 H9 human embryonic stem cells previously plated to 1:30 MATRIGEL® treated plasticware and grown in MEF conditioned media supplemented with 8 ng/ml of bFGF were LIBERASE treated and plated to surface modified plates in MEF conditioned media supplemented with 8 ng/ml of bFGF and not otherwise treated or supplemented with increasing concentrations of Y-27632. We observed that 24 and 48 hours after plating H9 human embryonic stem cells to surface modified plates small colonies could be observed on Surfaces 2-4 and 13, and CELLBIND™, and PRIMARIA™ (cat. no. 353846, Becton Dickinson, Franklin Lakes, N.J.) with crystal violet stain (FIGS. 24-26). Furthermore, the adherence of H9 human embryonic stem cell colonies was improved by the addition of Y-27632 and the effect was dose responsive (FIG. 25). Low concentrations of Y-27632 (1 to 2 micromolar) showed a minimal improvement in human ES cell attachment versus untreated human embryonic stem cells (FIG. 25) while higher concentrations of Y-27632 (4 to 20 micromolar) promoted adherence of human embryonic stem cells to surface modified plates as measured by crystal violet stain (FIGS. 25 and 26).

In addition to the dynamic regulation of human embryonic stem cell attachment by addition of Y-27632 to the cell culture media, we observed different rates of adhesion of human embryonic stem cells to various surface modified plastics in the presence of Y-27632. For example, cells were less adherent to CELLBIND™ plates and were more likely, over time, to detach from CELLBIND™ plates even in the presence of sustained Y-27632 treatment while cells were more adherent and less likely to detach from surface modified plates, 3, 4, or 13 or PRIMARIA™ when treated with the Rho kinase inhibitor, Y-27632 (FIGS. 25 and 26).

### Example 13

### Cells from the Human Embryonic Stem Cell Lines H1 and H9 Attach and Form Colonies at Different Rates on the Surface Modified Plates of the Present Invention in the Presence of Y-27632

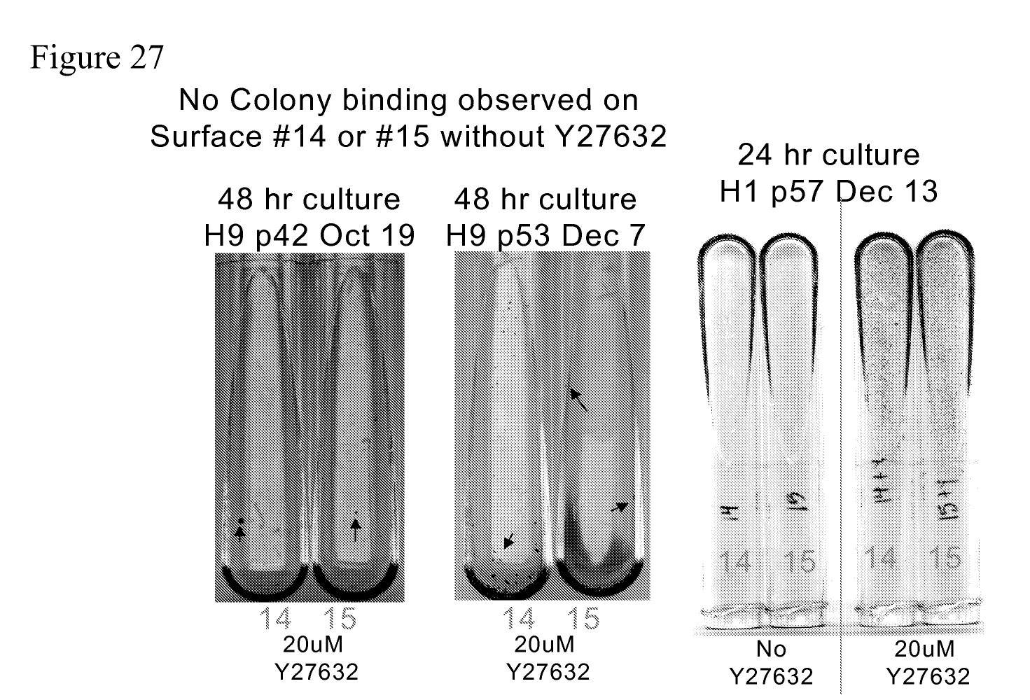

H1 and H9 human embryonic stem cells previously plated to 1:30 MATRIGEL®-treated plasticware and grown in MEF conditioned media supplemented with 8 ng/ml of bFGF were LIBERASE treated and plated to surface modified plates in MEF conditioned media supplemented with 8 ng/ml of bFGF and not otherwise treated or supplemented with 20 micromolar Y-27632. Forty-eight hours after plating H9 human embryonic stem cells to surface modified plates 14 and 15, small colonies were observed when the media was supplemented with 20 micromolar Y-27632 (attachment and colony formation was variable from experiment to experiment) (FIG. 27). H1 human embryonic stem cells also attached to and formed colonies on both Surface 14 and 15 in media supplemented with 20 micromolar Y-27632, and this was more prevalent than the binding observed with H9 human embryonic stem cells. These data indicate that there is human embryonic stem cell line-to-line variability in attachment to and colony formation on solid substrate surfaces.

### Example 14

### Human ES Cell Attachment to Surface Modified Plates Using Defined Media

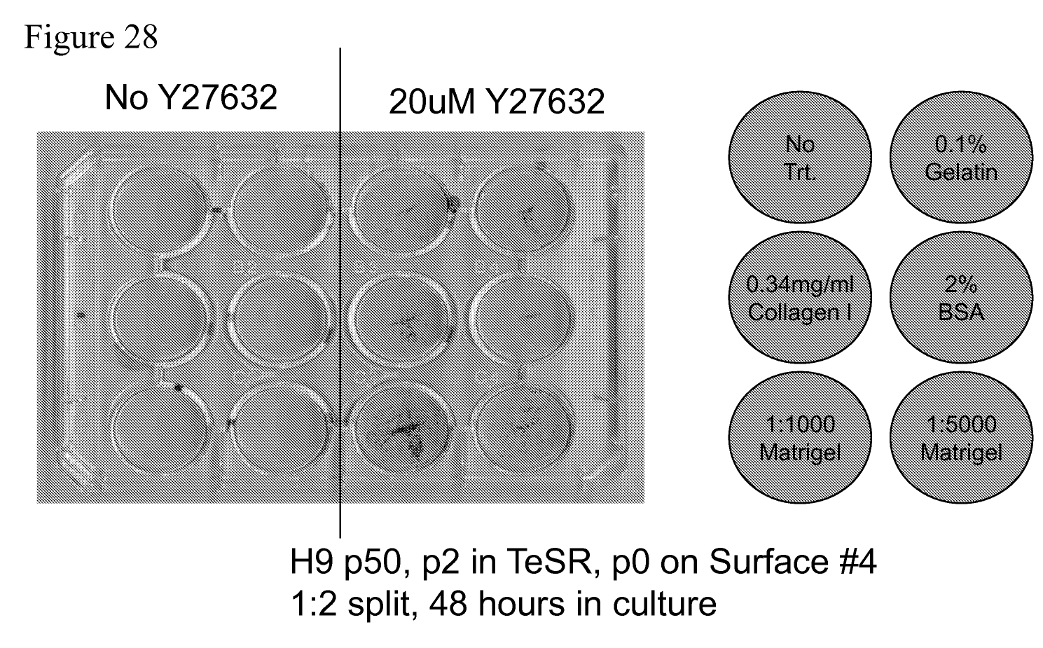

Passage 49 H9 human embryonic stem cells were passaged twice in the define media, mTeSR™, on MATRIGEL®-treated plasticware. The cells were then LIBERASE treated and plated onto the surface modified plate 4 in mTeSR™ media. Cells were either plated in media with or without 20 micromolar Y-27632. Wells were also treated with various proteins for 30 minutes prior to seeding cells (no treatment, 0.1% gelatin, 2% BSA, 0.34 mg/ml rat Collagen 1, 1:1000 MATRIGEL®, or 1:5000 MATRIGEL®) to determine if these proteins could promote human ES cell adhesion in defined media with or without Y-27632 (FIG. 28). We observed that in the absence of Y-27632, human embryonic stem cells plated onto a surface modified plate in defined media did not attach—even in the presence of extracellular matrix proteins such as Collagen I or 1:1000 MATRIGEL®. However, when 20 micromolar Y-27632 was added to define mTeSR™ media, human embryonic stem cells adhered to surface modified plate 4. Furthermore, this adherence was equivalent in untreated wells and wells treated with 0.1% gelatin, 2% BSA, and 0.34 mg/ml rat Collagen I. There was a modest increase in human embryonic stem cell attachment in wells with low concentrations of MATRIGEL® (1:1000 and 1:5000 dilutions), however these concentrations of MATRIGEL® were insufficient to promote adhesion in the absence of Y-27632. These results demonstrate that in the presence of the ROCK Inhibitor, Y-27632, human embryonic stem cells can be cultured on modified plastic substrates in defined media and that low concentrations of MATRIGEL® of about 1:1000 or 1:5000 can improve this adhesion.

### Example 15

### Surface Modified Plates in a Flask Format can Promote Human Embryonic Stem Cell Attachment and Differentiation to Definitive Endoderm and Pancreatic Endoderm

H1 and H9 human embryonic stem cells previously plated to 1:30 MATRIGEL®-treated plasticware and grown in MEF conditioned media supplemented with 8 ng/ml of bFGF were LIBERASE treated and plated to T25, T75, T150, and T175 flasks at a 1:2 or 1:3 seeding density onto various size flasks with modified surfaces. The cells were seeded in MEF conditioned media supplemented with 8 ng/ml of bFGF and 20 micromolar Y-27632. Human embryonic stem cell colonies were then allowed to grow, with daily media changes of MEF conditioned media supplemented with 8 ng/ml of bFGF and 20 micromolar Y-27632, until the plates were approximately 50% confluent. At this time the media was changed to DMEM/F12 media containing 2% BSA, 100 ng/ml Activin A, 20 ng/ml Wnt3a, and 20 micromolar Y-27632 and the cells were maintained in this media for 2 days with daily media changes. On day 3 and 4 the media was changed to DMEM/F12 media containing 2% BSA, 100 ng/ml Activin A, and 20 micromolar Y-27632. Cells were then released from the surface with TrypLE and assays by flow cytometry for expression of the definitive endoderm (DE) surface marker, CXCR4. We observed that under these conditions, human embryonic stem cells differentiated to a highly CXCR4 positive population, that was as high as almost 90% CXCR4+, indicating that the cells were mostly differentiated to definitive endoderm (Table 5). Furthermore, the attachment of the cells to the culture surface during growth or during differentiation was dependent on maintaining ROCK inhibition, since withdrawal of Y-27632 from the culture media resulted in cell detachment from the plastic.

We wished to determine if pancreatic endoderm could be formed from the definitive endoderm derived on surface modified plates in flask format. To do so, we incubated the cells for an additional four days with Y-27632 (20 micromolar), FGF7 (50 ng/ml), KAAD cyclopamine (2.5 micromolar), and 1% B27 (Invitrogen) in DMEM-F12 and then an additional four days in this media supplemented with Retinoic Acid (1 micromolar; Sigma-Aldrich) to differentiate the cells to a pancreatic endoderm stage. RNA samples were then taken and analyzed by real-time PCR for the pancreatic marker PDX1. Treated samples were normalized to untreated controls set to a fold change of 1. We observed that samples had increased levels of PDX1 versus undifferentiated human embryonic stem cells; with mRNA levels at least 256 fold higher in the differentiated cells than that observed in undifferentiated human embryonic stem cells.

### Example 16

### Surface Treatment and Surface Modified Plates

Surface modified plates were prepared by treating injection molded items using a corona plasma treatment or a microwave plasma treatment (Table 6). The polymer materials used in injection molding were polystyrene, polycarbonate, a blend of polycarbonate and polystyrene, and cyclic olefin copolymer. The surface modified plates were individually packed in plastic bags, then sterilized by gamma irradiation (25 kGy), and finally stored at room temperature until used in cell culture or surface characterization experiments. Surface modified plates 18, 30 and 31-32 were molded using the same polymer materials as surface modified plates 19, 33 and 34, respectively, but were not plasma treated. Surfaces 14 and 31 were not gamma irradiated.

Corona plasma treatment was carried out in a metal vacuum chamber with only one electrode inside the chamber and electrically isolated from the inside of chamber (C-Lab Plasma; Vetaphone A/S, Denmark). The metal walls served as counter electrode (ground). A self-tuning corona generator generated the electrical field giving sufficient energy to generate plasma in the entire chamber. An item to be treated was placed at the bottom of the chamber. The chamber was closed and evacuated to a pressure of 10⁻² mbar. At this pressure the valve to the vacuum pump was closed and the corona generator engaged. The generator was set to generate an output of 2000 W. The plasma was energized for 5 to 60 seconds. The gas inlet valve (air) was then opened, and the pressure in the chamber returned to atmospheric level.

The microwave plasma treatment was carried out in a quarts vacuum chambers (Model 300-E for surface modified plates 5-12 and Model 440 for surface modified plates 14 and 15; both from Technics Plasma GmbH, Germany). The energy to generate the plasma was supplied by a 2.43 GHz microwave generator outside the chamber. An item to be treated was placed on a glass plate inside the chamber. The chamber was closed and evacuated to a pressure between 0.3 and 0.5 mbar. The valve to the vacuum pump was kept open, and the pressure was maintained at the desired value by adjusting gas (air or oxygen) flow with the gas inlet valve. The microwave generator was then engaged. The generator was set to generate an output of 500 or 600 W. The pump valve was then closed, and the air inlet valve was opened, in order to bring the pressure in the chamber to atmospheric level.

Table 6 shows power, time, pressure, and gasses used in preparing surface modified plates by corona plasma or microwave plasma.

### Example 17

### Surface Characterization of the Surface Modified Plates of the Present Invention

### Water Contact Angles

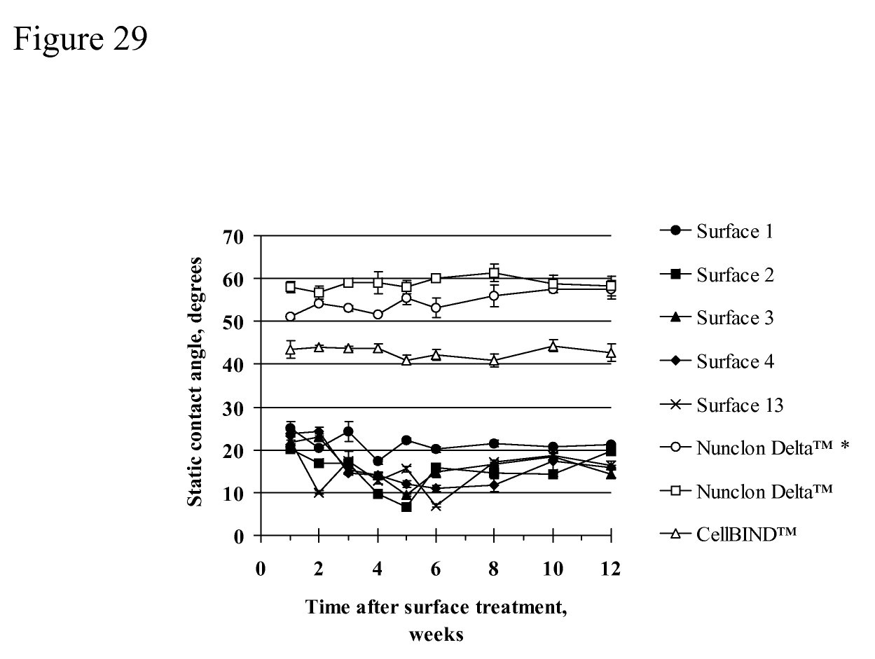

Surface modified plates 1-4 and 13 were individually packed in plastic bags, sterilized, and stored at room temperature throughout the test period. Contact angles were first measured one week after surface treatment and sterilization, and then again at the time points given in FIG. 29. All contact angle measurements were done using the static sessile drop method and a PG-X measuring Head from FIBRO Systems AB, Sweden [goniometer consisting of video camera and computer software (v. 3.1)]. The tangent leaning method was used for calculation of the contact angles. Drops of 4.0 μL MilliQ water was applied using automatic drop application in static mode, according to the manufactures instructions. The contact angle of each drop was measured once (7 drops were applied to each sample per time point). For each time point, a new sample was used in order to avoid any influence from earlier measurements. Measurements on NUNCLON DELTA™ and CELLBIND™ surfaces was performed under the same experimental conditions as measurements on Surface 1-4 and 13, but the surface treatment and sterilization was done more than 12 weeks before the first measurement (NUNCLON DELTA™* was sterilized one week before the first measurement). FIG. 29 shows that surface modified plates 1-4 and 13 were of similar hydrophilicity and more hydrophilic (lower water contact angles) than NUNCLON DELTA™ and CELLBIND™ surfaces. The hydrophilicity of surface modified plates 1-4 and 13 was stable for at least 12 weeks after surface treatment and sterilization.

CELLBIND™ has previously been described as having a contact angle of 13.4 degrees (standard deviation of 4 degrees) [Corning Technical Report (2005), Corning® CELLBIND® Surface: An Improved Surface for Enhanced Cell Attachment (CLS-AN-057 REV1) on http://catalog2.corning.com/Lifesciences/media/pdft_CellBIND_Improved_Surface_CLS_AN_{—}057.pdf].

### Negative Charge Density

The density of negative charges on surface modified plates 1-4 and 13, NUNCLON DELTA™ surface, CELLBIND™ surface, PRIMARIA™ surface, FALCON™ surface, and a non-treated (but sterilized) polystyrene surface (all in 3-cm dish format) was determined. Three ml of aqueous crystal violet solution (0.015% w/v) was dispensed in each dish, and dishes were incubated for 60 minutes at room temperature under gentle shaking (50 rpm). In order to remove crystal violet not bound to the surfaces, the dishes were washed three times with 3 ml MilliQ water, and then dried over night at 60° C. The crystal violet bound to the surface was desorbed by addition of 1.5 ml of 0.1 M HCl in EtOH solution (99%) and incubating the dishes for 2 minutes at room temperature under gentle shaking (50 rpm). Absorbance of the HCl:EtOH solution with desorbed crystal violet was measured at 590 nm using an EnVision 2100 microplate reader (Perkin Elmer; Waltham, Mass., USA). Absorbance values were corrected for background absorbance of HCl:EtOH solution. The negative charge density was measured on three dishes per surface, and absorbance measurement was performed in triplicate for each dish.

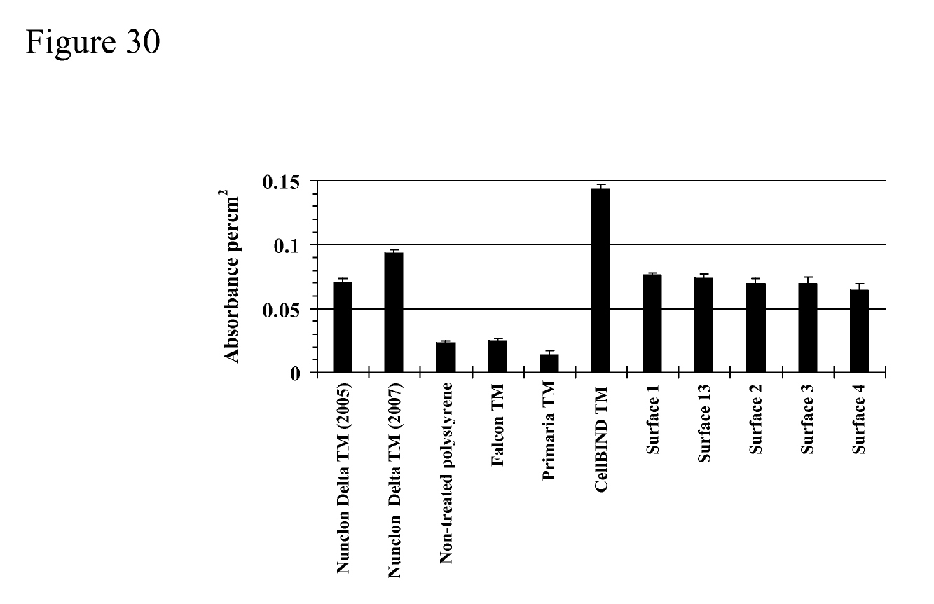

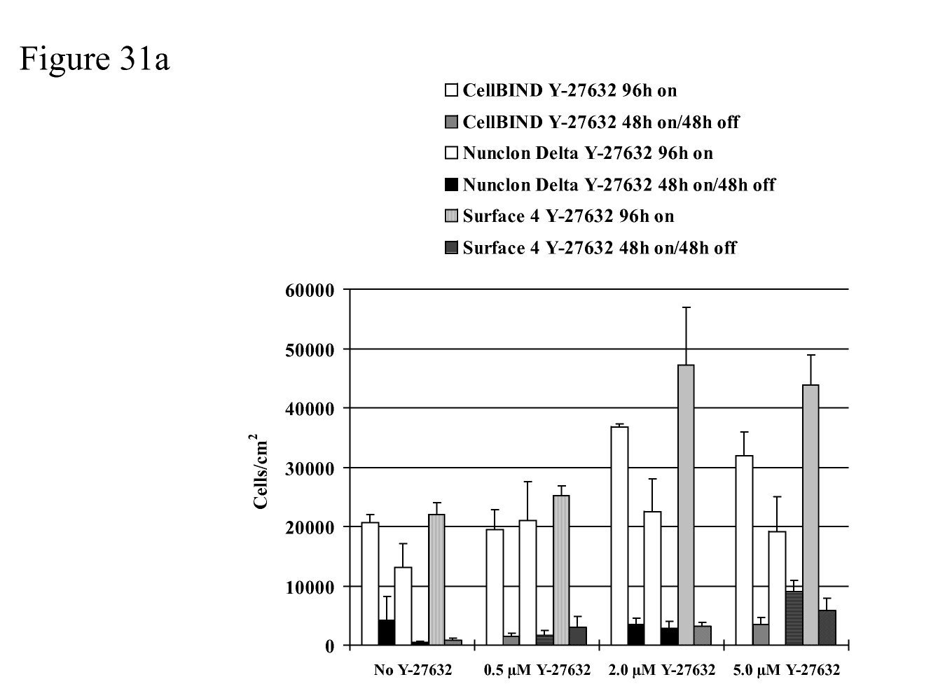

The negative charge density for surface modified plates is shown in FIG. 30. The negative charge densities of Surfaces 1-4 and 13 were similar, but longer surface treatment time in the interval of 5-60 seconds tended to result in a lower surface negative charge density. Surfaces 1-4 and 13 had significantly lower negative charge densities than CELLBIND™ surface and the NUNCLON DELTA™ surface treated in 2007. Surfaces 1-4 and 13 had negative charge densities at the same level as the NUNCLON DELTA™ surface treated in 2005, and significantly higher negative charge densities than the PRIMARIA™ surface, the FALCON™ surface, and a non-treated (but sterilized) polystyrene surface. The lower negative charge density of the NUNCLON DELTA™ surface treated in 2005 than that of the NUNCLON DELTA™ surface treated in 2007, suggest that surface-treated polystyrene becomes slightly less negatively charged over time. The high level of negative charge density of CELLBIND™ is not because of higher surface roughness and thus surface area (See AFM analysis in this Example).

### X-Ray Photoelectron Spectroscopy (XPS)

Surface modified plates 1-4 and 13-15, and plates with NUNCLON DELTA™, COSTAR™, FALCON™, CELLBIND™ and PRIMARIA™ surfaces were analyzed using XPS. Sample was presented to the x-ray source by cutting sections from the plates and mounting them with spring clips onto a stainless steel sample holder. Samples were irradiated with A1 kα radiation (1486 eV). The analysis was performed with an angle of 45° between the sample and analyzer. The spectra were curve fit using the software package provided by the instrument's vendor, Physical Electronics. The software utilized commercial MATLAB™ routines for data processing. The instrument used for the analysis was a Physical Electronics Model 5400 X-Ray Photoelectron Spectrometer. The outermost two to five nanometers in depth in a region of about one millimeter in diameter from the surface treated part of the plates was analyzed in each of two plates per surface.

Surface elemental composition in units of atomic percent is shown in Table 7. All surface modified plates contained carbon, oxygen and nitrogen (hydrogen is not detected in XPS) in the surface. Surfaces 1-4, Surface 13 and CELLBIND™ surface contained more oxygen than the other surfaces analyzed. Surfaces 1-4 and Surfaces 13-15 contained less nitrogen than PRIMARIA™, but more nitrogen than the surfaces of NUNCLON DELTA™, COSTAR™, FALCON™, and CELLBIND™ plates. Oxygen and nitrogen levels correlated positively with longer surface treatment time (Surfaces 1-4 and 13), and the highest levels of both of these elements were obtained using 30 or 60 seconds of corona plasma treatment (Surface 3 and Surface 4, respectively). Surfaces 3 and 4 were similar in elemental composition. Surfaces 2 and 13 were similar in elemental composition, and more like Surfaces 3 and 4 than Surface 1 in elemental composition.

C1s spectra peaks were curve fit (best chi-squared fit), in order to identify and quantify the bonding environments for carbon in the surfaces, by using peak widths and energy locations for species as found in the literature (Table 8). The concentrations are reported in units of atomic percent, which were obtained by multiplying the area percent by the atomic concentration. Surfaces 2-4 and 13 were similar in terms of the carbon bonding environments. The proportion of carbon in C*—C—O—C—C* bonding environment was lower in Surfaces 2-4 and 13 than in the other surfaces analyzed. The proportion of carbon in O—[C═O]—O bonding environment was higher in Surfaces 2-4 and 13 than in the other surfaces analyzed. Similarities between Surfaces 2-4 and 13 and Surface 1, CELLBIND™ surface, and/or PRIMARIA™ surface were also identified. The proportion of carbon in C—O—C or C—NH₃⁺ bonding environment (same energy location in spectra) was higher in Surfaces 1-4 and 13 than in the other surfaces analyzed. The proportion of carbon in C—O—C*═O bonding environment was higher in Surfaces 2-4, Surface 13, and PRIMARIA™ surface than in the other surfaces analyzed. The proportion of carbon in CO₃— bonding environment was higher in Surfaces 2-4, Surface 13, and CELLBIND™ surface than in the other surfaces analyzed. The proportion of carbon in C═O bonding environment was higher in Surfaces 1-4, Surface 13, and CELLBIND™ surface than in the other surfaces analyzed. The proportion of carbon in C—[O]—C bonding environment was higher in Surfaces 1-4, Surface 13, CELLBIND™ surface, and PRIMARIA™ surface than in the other surfaces analyzed. The energy loss peak resulted from an aromatic Π→Π* transition, and is an indicator of surface aromaticity.

The O1s spectra peaks were almost Gaussian and could not be curve fit. N1s spectra peaks were curve fit (best chi-squared fit), in order to identify and quantify the bonding environments for nitrogen in the surfaces, by using peak widths and energy locations for species as found in the literature (Table 9). The concentrations are reported in units of atomic percent, which were obtained by multiplying the area percent by the atomic concentration. The N1s signals from NUNCLON DELTA™, CELLBIND™, COSTAR™, and FALCON™ surfaces were weak, and it was, therefore, not possible to do identification of the bonding environments for nitrogen in these surfaces. N1s spectra were indistinguishable for surface modified plates 1-4 and 13, and data resulting from curve fitting of two representative Nls spectra is shown. The proportion of nitrogen in —NH₃⁺ bonding environment was higher in Surfaces 1-4 and 13 than in Surfaces 14 and 15 and PRIMARIA™ surface. Nitrogen in —NH₂ bonding environment was detected only in Surfaces 14 and 15 and PRIMARIA™ surface. Nitrogen in —NO₂ bonding environment was detected only in Surfaces 1-4 and 13, and in a single sample of Surface 15. Nitrogen in NO₃ bonding environment was detected only in Surface 15 and PRIMARIA™ surface.

CELLBIND™ has previously been described as having an elemental composition of 70.4% carbon, 29.0% oxygen, 0.6% nitrogen, and <0.01% other elements, and a relatively high concentration of C—[O]—C, C═O, and COOH/R groups, as analyzed by ESCA [Corning Technical Report (2005), Corning® CellBIND® Surface: An Improved Surface for Enhanced Cell Attachment (CLS-AN-057 REV1) on http://catalog2.corning.com/Lifesciences/media/pdf/t_CellBIND_Improved_Surface_CLS_AN_{—}057.pdf].

PRIMARIA™ has previously been described as having an elemental composition of 74.6% carbon, 14.1% oxygen, 11.1% nitrogen, and 0.2% other elements, with mainly nitrile (C≡N) and urea [HN(C═O)NH]carbon-to-nitrogen bonding environments, as analyzed by ESCA.

### Atomic Force Microscopy (AFM)

Surface modified plates 1-4 and 13, and plates with NUNCLON DELTA™, or CELLBIND™ surfaces were analyzed using AFM. Samples were analyzed using a Digital Instruments Multimode Atomic Force Microscope in tapping mode. The tip used was a tapping mode tip, type TESP7. Samples were attached to the sample disks with double sticky tape. Regions of 10 μm×10 μm and 500 nm×500 nm of the surface-treated part of the plates were analyzed. Surface mean roughness (Ra) and maximum height (Rmax) in units of nanometers are shown in Table 10. Like the plates with NUNCLON DELTA™ and CELLBIND™ surfaces, surface modified plates 1-4 and 13 were relatively smooth, and Ra and Rmax did not correlate with surface treatment time in either of the two scans. Analysis of non-treated polystyrene and oxidized polystyrene surfaces intended for cell culture, and PRIMARIA™ surface has been described by Shen and Horbett (J. Biomed. Mater. Res. 57:336-345, 2001): surface roughness approximately 4 nm for all three surfaces.

### Example 18

### Surface Elemental Composition and Contact Angle in Relation to Human ES Cell Attachment and Colony Formation

A summary of the results of the XPS analysis of surface elemental composition, the surface contact angle measurements, and human embryonic stem cell attachment and colony formation experiments is given in Table 11.

Human embryonic stem cell attachment to and colony formation (at least 15 colonies per 10 cm² surface) on a solid substrate surface in the absence of a compound capable of inhibiting Rho or Rho kinase was observed on only surface modified plates 2, 3, 4, 13, CELLBIND™ plates, and PRIMARIA™ plates (cells were presented to the surfaces as a suspension of clusters of cells). Surface modified plates 2-4 and 13 supported cell attachment, colony formation and passaging. After about three passages, the growth rate of human embryonic stem cells on surface modified plates 2-4 and 13 declined spontaneously (only in the absence of Rho inhibition and Rho kinase inhibition), although cell morphology indicated that the cells were not differentiating. Furthermore, pluripotency marker expression was maintained in cells passaged four times on Surface 3. CELLBIND™ plates supported human embryonic stem cell attachment and colony formation, but differentiation of the cells was observed prior to the first passage. Based upon cell morphology observations, PRIMARIA™ plates supported human embryonic stem cell attachment and colony formation, without signs of differentiation (passaging was not tested). Both oxygen (for example, Surface 2 versus Surface 14) and nitrogen (for example, PRIMARIA™versus COSTAR™; and Surfaces 2 and 13 versus CELLBIND™) content of surfaces had an effect on the ability of the surfaces to support human embryonic stem cell attachment and colony formation in the absence of Rho inhibition and Rho kinase inhibition. Surfaces with a nitrogen content of at least about 0.9%, a sum of nitrogen and oxygen content of at least about 22.3%, and a water contact angle of at least about 13.9 degrees supported human embryonic stem cell attachment and colony formation in the absence of Rho inhibition or Rho kinase inhibition.

Human embryonic stem cell attachment and colony formation (at least 15 colonies per 10 cm² surface) on a solid substrate surface in the presence of a compound capable of inhibiting Rho or Rho kinase was observed on surface modified plates 1-15, surface modified plate 19, surface modified plate 33, surface modified plate 34, CELLBIND™, and PRIMARIA™ (cells were presented to the surfaces as a suspension of clusters of cells). We noted that surfaces 2-4 and 13 and PRIMARIA™were better than surfaces 1, 19, 33, 34 and CELLBIND™, which again were better than surfaces 5-12, 14 and 15, at promoting human embryonic stem cell attachment and colony formation. On surface modified plates 3 and 4, and in the presence of a Rho kinase inhibitor, human embryonic stem cells attached and formed colonies that expanded and could be passaged at least 10 times, giving rise to pluripotent cells with normal karyotype (karyotype tested only in cells grown on Surface 4). Both oxygen (for example, CELLBIND™ versus NUNCLON DELTA™) and nitrogen (for example, PRIMARIA™ versus COSTAR™; and Surfaces 2 and 13 versus CELLBIND™) content of surfaces had an effect on the ability of the surfaces to support human embryonic stem cell attachment and colony formation in the presence of Rho kinase inhibition. Surfaces with a nitrogen content of at least about 0.5%, a sum of nitrogen and oxygen content of at least about 17.2%, and a water contact angle of at least about 13.9 degrees supported human embryonic stem cell attachment and colony formation in the presence of Rho kinase inhibition. Surfaces with a nitrogen content of at least about 0.5%, a sum of nitrogen and oxygen content of at least about 17.3% but less than 19.9%, and a water contact angle of at least about 9.4 degrees supported human embryonic stem cell attachment and colony formation in the presence of Rho kinase inhibition in some cases (surface 14), but not in others (surfaces 22-24).

We noted that removal of Rho kinase inhibitor from human embryonic stem cell cultures cultured on surface modified plate 4 resulted in detachment of the human embryonic stem cells from the surface of the solid substrate. The cells could then be reattached to the surface by re-treatment with a Rho kinase inhibitor. Given that enzymatic passage of human embryonic stem cells is a potential stressor and may cause karyotypic instability, using temporary removal of Rho kinase inhibitor to passage human embryonic stem cells could eliminate the stresses of enzymatic passage.

Human embryonic stem cell attachment and colony formation was also demonstrated using animal-component-free medium, Rho kinase inhibition and surface modified plate 4. Pre-treatment of surface modified plate 4 with extracellular matrix proteins resulted in more colonies, but only in the presence of Rho kinase inhibition.

In addition to passaging human embryonic stem cells with enzymatic methods that maintain colony style culture conditions by passaging cells as clusters, human embryonic stem cells could also be passaged as single cells using enzymes like TrypLE™ or ACCUTASE™. In the presence or in the absence of Rho kinase inhibitor, human embryonic stem cell colonies dissociated into a suspension of single cells using TrypLET™ attached to surface modified plates 3 and 4, and formed colonies that could be passaged at least five times and give rise to cells with pluripotency markers.

Removal of Rho kinase inhibitor from the human embryonic stem cell cultures prepared by passaging the cells as a suspension of single cells did not result in detachment of the human embryonic stem cells from the surface of the solid substrate, but resulted in colonies that grew faster than if the Rho kinase inhibitor was not removed.

### Example 19

### Treatment with Y-27632 Enhance HEK293 Cell Attachment to the Surface Modified Plates of the Present Incention

Human embryonic kidney cells 293 (HEK293, ECACC no. 85120602) were maintained in Eagle's Minimum Essential Medium (EMEM; Lonza, Verviers, Belgium) containing 10% fetal bovine serum (FBS; Lonza). The cells were adapted to Pro293a-CDM medium (Lonza), a chemically defined, serum-free medium optimized for cultivation of adherent HEK293, by gradually and over several passages using the sequential ratios of 3:1, 1:1, 1:3, 1:7, and finally 0:1 of serum-supplemented EMEM and Pro293a-CDM medium. For maintenance and adaptation, HEK293 cells were seeded at 2.0×10⁴ cells/cm² in 75-cm² flasks with NUNCLON DELTA™ surface (Thermo Fisher Scientific, Roskilde, Denmark) and passaged at 70-80% confluence using Trypsin/EDTA for dissociation.

Pro293a-CDM medium (100 μl) supplemented with Y-27632 (Sigma Chemical Co., St. Louis, Mo.) in concentrations of 1.0, 4.0 or 10 μM was dispensed in flat-bottomed, 96-well plates with either Surface 4, NUNCLON DELTA™, or CELLBIND™ surfaces. Another 100 μl of Pro293a-CDM medium with HEK293 cells was added to the wells (4.0×10⁴ cells/cm²). The cultures were then incubated at 37° C. in a humidified atmosphere of 5% CO₂ in air for: (i) 96 hours; or (ii) 48 hours, followed by washing cultures once with 200 μl Dulbecco's Phosphate Buffered Saline (DPBS; Lonza), then adding 200 μl of Pro293a-CDM medium without Y-27632, and finally incubating cultures for another 48 hours.

The number of viable cells in the wells was then determined using a lactate dehydrogenase (LDH) activity kit from Roche, Switzerland. Briefly, wells were washed with Pro293a-CDM medium, and adherent cells were lysed in 100 μl DPBS with 2% (v/v) Triton X-100 (Sigma Chemical Co.) during a 30-min incubation at 37° C. Lysate and 100-μl catalyst and dye reagent mixture were mixed and incubated in the dark at 25° C. for 30 min. The reaction was stopped by adding 50 μl of 1.0 M HCl, and the absorbance at 490 nm was measured in a microplate reader (Genios Pro; Tecan, Austria). The number of cells was calculated using the A490 values from these samples and from standards containing LDH from a known number of cells.

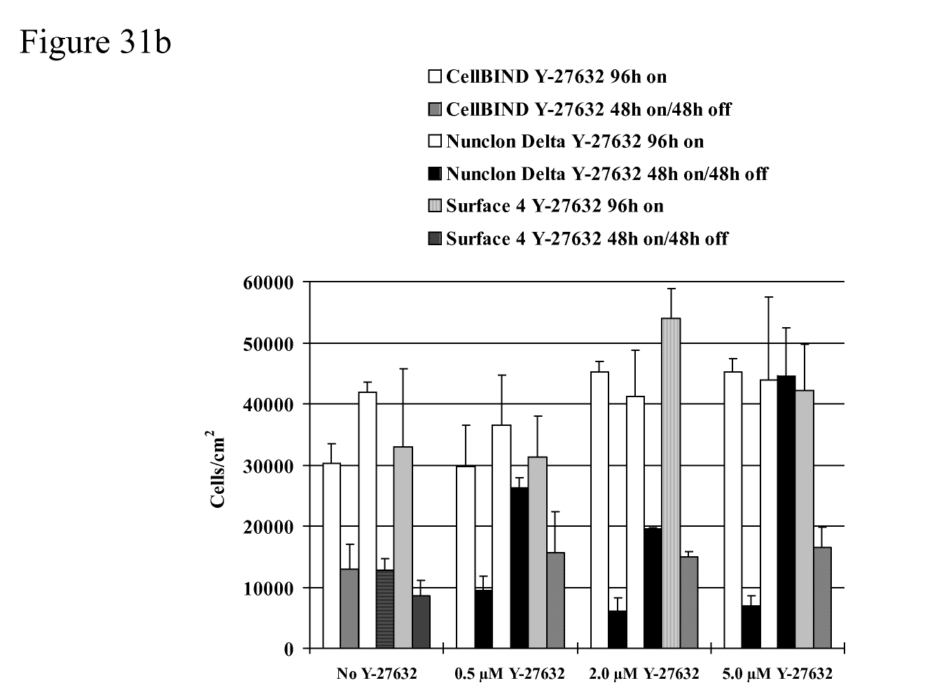

The effect of the solid substrate surfaces and Y-27632 on attachment and growth of HEK293 cells in Pro293a-CDM medium is shown in FIG. 31a, where the 96-hour continuous exposure to Y-27632 is labeled “Y-27632 96 h on” and the 48-hour continuous exposure to Y-27632 followed by a change of medium and 48 hours of incubation in the absence of Y-27632 is labeled “Y-27632 48 h on/48 h off'. In the absence of Y-27632, HEK293 cells attached to all three surfaces. A change of medium after 48 hours of incubation resulted in significantly fewer cells in the cultures, measured after 96 hours of incubation. Y-27632 enhanced attachment of HEK293 cells on Surface 4 and CELLBIND™ surface when applied at concentrations of 2.0 and 5.0 μM. Removing Y-27632 after 48 hours of incubation resulted in significant detachment of cells from all three surfaces.

A similar experiment, but using 2.0×10⁴ non-adapted HEK293 cells per cm² and EMEM supplemented with 10% FBS throughout, was performed. The effect of the solid substrate surfaces and Y-27632 on attachment and growth of HEK293 cells in EMEM supplemented with 10% FBS is shown in FIG. 31b, where the 96-hour continuous exposure to Y-27632 is labeled “Y-27632 96h on” and the 48-hour continuous exposure to Y-27632 followed by a change of medium and 48 hours of incubation in the absence of Y-27632 is labeled “Y-27632 48 h on/48 h off”. In the absence of Y-27632, HEK293 cells attached to all three surfaces. A change of medium after 48 hours of incubation resulted in significantly fewer cells in the cultures, measured after 96 hours of incubation. Y-27632 enhanced attachment of HEK293 cells on Surface 4 and CELLLBIND™ surface when applied at concentrations of 2.0 and 5.0 μM. Removing Y-27632 after 48 hours of incubation resulted in significant detachment of cells from Surface 4 and CELLBIND™.

### Example 20

### Treatment with Y-27632 and H-1152 Enhance HEK293 Cell Growth on Surface Modified Plates

HEK293 cells were maintained in EMEM (Lonza) containing 10% FBS (Lonza). Cells were passaged at 70-80% confluence using Trypsin/EDTA for dissociation, and seeded at 2.0×10⁴ cells/cm² in 75-cm² flasks with NUNCLON DELTA™ surface (Thermo Fisher Scientific, Roskilde, Denmark).

EMEM (500 μl) supplemented with 10% FBS containing 1.0, 5.0, 10, 15 or 20 μM Y-27632 (Sigma Chemical Co.), or 0.4, 1.2, 1.6, 2.4 or 2.8 μM H-1152 (Calbiochem, EMD Chemicals Inc., Darmstadt, Germany) was dispensed in Multidish 24-well plates with either Surface 4 or a non-treated (but gamma irradiated; 25 kGy) polystyrene surface. Another 500 μl of EMEM supplemented with 10% FBS and containing HEK293 cells were added to the wells (2.0×10⁴ cells/cm²). The cultures were placed in an IncuCyte™ Plus (Essen Instruments, Michigan, USA) and incubated at 37° C. in a humidified atmosphere of 5% CO₂ in air. The IncuCyte™ Plus is an automated imaging platform, configured to fit inside a CO₂ incubator, and designed to provide kinetic, non-invasive live cell imaging by acquiring phase contrast images of the cells at user-defined times and locations within the cultures.

The primary metric of the instrument is culture confluence, that is, the fraction of the surface that is covered by cells. The HEK293 cells were incubated for 72 hours without manipulations, and images were collected every two hours at 9 positions in triplicate cultures. Culture confluence was determined using the IncuCyte™ Plus software (v. 3.4.1.25966).

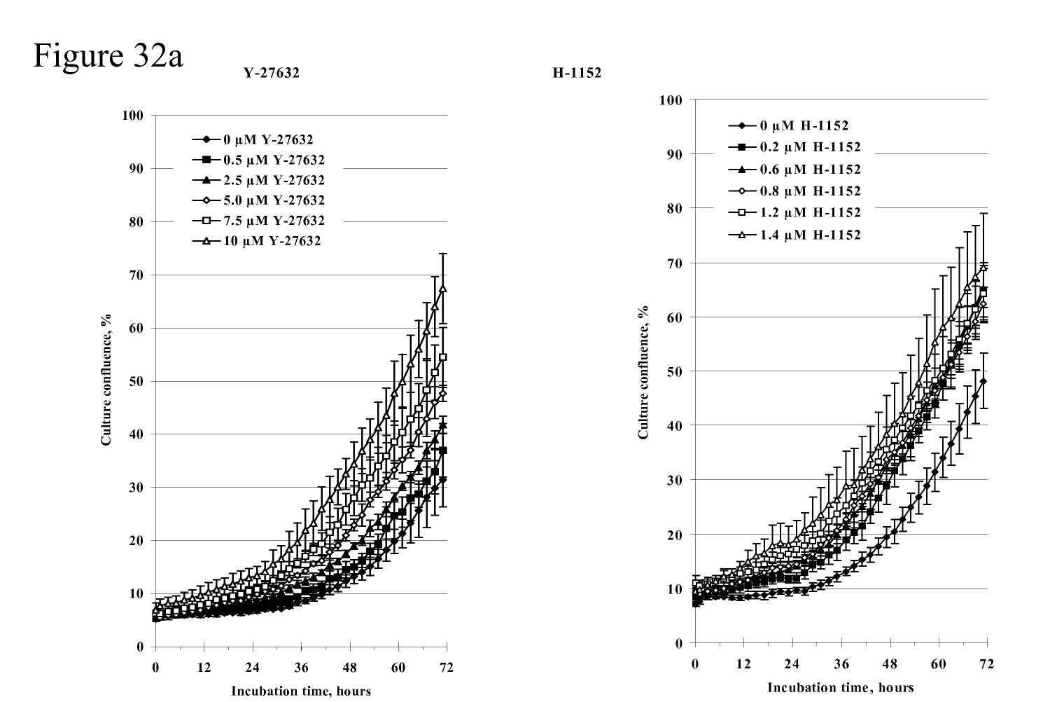

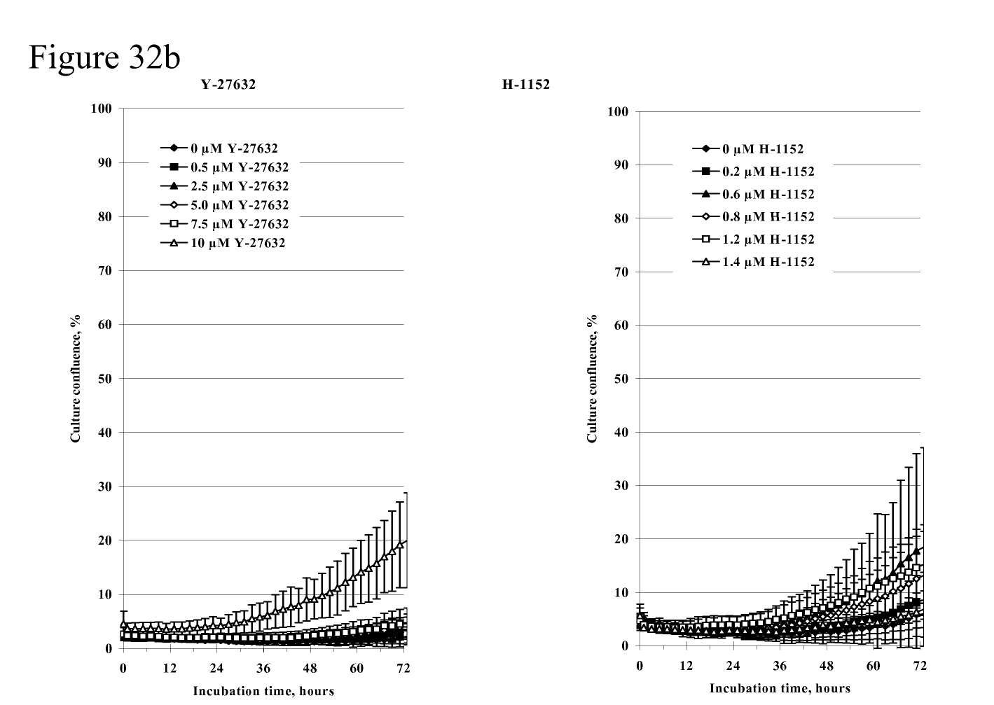

Increasing concentration of Y-27632 and H-1152 enhances attachment and growth of HEK293 cells on Surface 4 (FIG. 32a). The effect of a non-treated cell culture surface and Y-27632 or H-1152 on attachment and growth of HEK293 is shown in (FIG. 32b). Growth and attachment of HEK293 cells was slightly enhanced in the presence of 10 μM Y-27632 and 0.6-1.2 μM H-1152. However, the enhancement of growth and attachment of HEK293 cells on a non-treated cell culture surface is insignificant in comparison to Surface 4.

### Example 21

### Treatment with H-1152 Enhances HEK293 Cell Growth and Attachment to Surface Modified Plates

HEK293 cells were maintained in EMEM (Lonza) containing 10% FBS (Lonza). Cells were passaged at 70-80% confluence using Trypsin/EDTA for dissociation, and seeded at c 2.0×10⁴ cells/cm² in 75-cm² flasks with NUNCLON DELTA™ surface (Thermo Fisher Scientific, Roskilde, Denmark).

EMEM (1.0 ml) supplemented with 10% FBS containing 0.4, 0.8, 1.2, 1.6, 2.0, 2.4 or 2.8 μM H-1152 was dispensed in Multidish 12-well plates with Surface 4. Another 1.0 ml of EMEM supplemented with 10% FBS and containing HEK293 cells were added to the wells (4.0×10⁴ cells/cm²). The cultures were placed in an IncuCyte™ Plus, and incubated at 37° C. in a humidified atmosphere of 5% CO₂ in air for 42 hours (images were collected every 6 hours). One ml of culture medium was then removed by pipetting, and 1.0 ml EMEM supplemented with 10% FBS containing 0.2, 0.4, 0.6, 0.8, 1.0, 1.2 and 1.4 μM H-1152 was added. The cultures were placed in the IncuCyte™ Plus again, and images were collected every hour over the following 25 hours. Images were collected at 9 positions in triplicate cultures, and culture confluence was determined using the IncuCyte™ Plus software. Images from the IncuCyte™ Plus collected at specific positions in HEK293 cell cultures grown in the absence or presence of H-1152 (0.6 μM) was retrieved and presented as phase-contrast micrographs for the comparison of HEK293 culture morphology at the following time points: start of incubation (0 hours), just before medium change (42 hours), 1 hour after the medium change (43 hours), and, finally, after 52 hours of incubation.

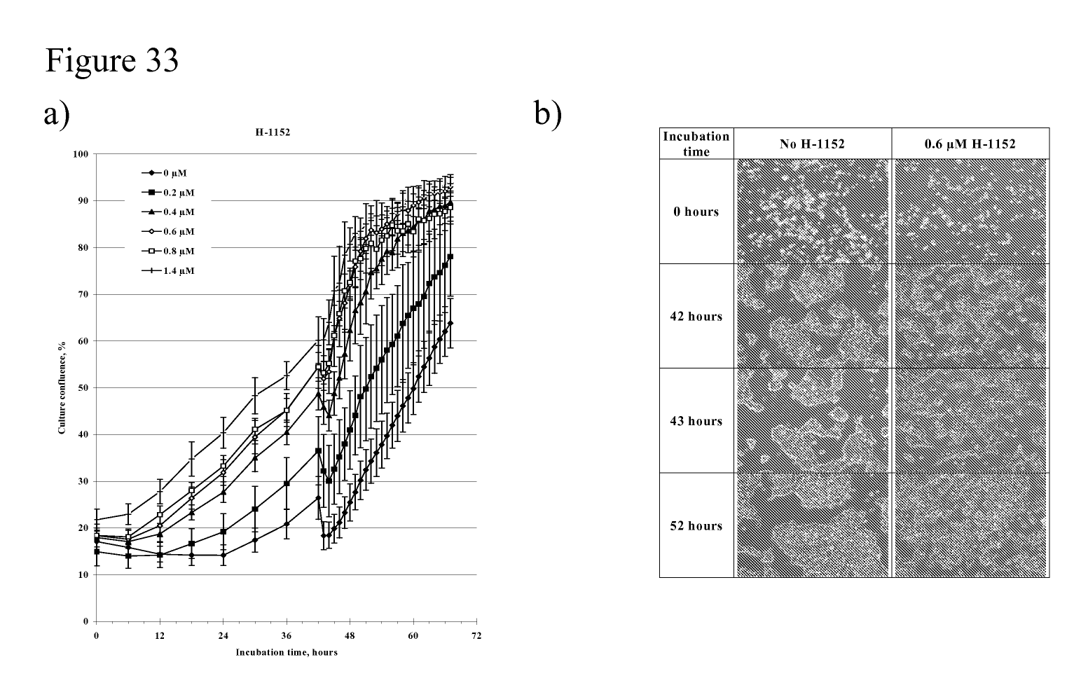

In the absence of H-1152 and in the presence of 0.2 μM or 0.4 μM H-1152, the change of 50% of the medium after 42 hours of incubation resulted in a significant reduction in culture confluence (FIG. 33a). In the presence of 0.6 μM, 0.8 μM or 1.4 μM H-1152, the effect of changing the medium was minimal HEK293 cells grown on Surface 4 in the presence of H-1152 covered the solid substrate surfaces more evenly than HEK293 cells grown on Surface 4 in the absence of H-1152 (FIG. 33b). In the absence of H-1152, HEK293 cells formed large clusters, whereas, HEK293 cells in the presence of H-1152 formed smaller clusters with lower cell density.

### Example 22

### Treatment with Y-27632 Enhances HEK293 Cell Growth Over Three Passages on the Surface Modified Plates of the Present Invention

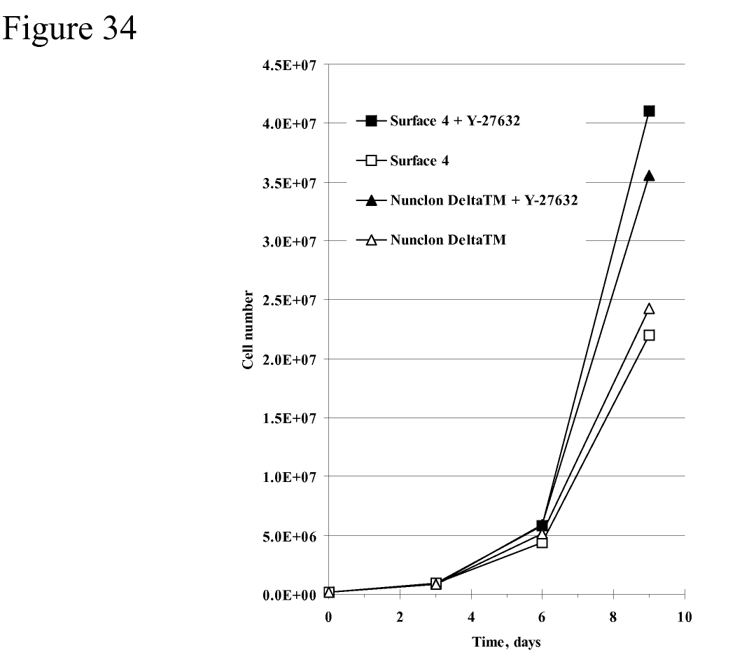

EMEM (500 μl) supplemented with 10% FBS containing 5.0 μM Y-27632 was dispensed in wells of Multidish 24-well plates with either Surface 4 or NUNCLON DELTA™ surface. Another 500 μl of EMEM supplemented with 10% FBS and containing HEK293 cells was added to the wells (2.0×10⁴ cells/cm²), and the cultures were incubated at 37° C. in a humidified atmosphere of 5% CO₂ in air for 3 days. Cells were passaged by treatment with Trypsin/EDTA (Lonza, Verviers, Belgium) for two minutes at 37° C., and the total cell number was determined using a NucleoCount Cell Counter (Chemometec A/S, Allerød, Denmark). For successive passages, HEK293 cells were seeded at 2.0×10⁴ cells/cm². The growth of HEK293 cells on Surface 4 and NUNCLON DELTA™ surface was enhanced by the presence of 2.5 μM Y-27632 (FIG. 34).

### Example 23

### Attachment, Cultivation and Maintenance of Human Embryonic Stem Cells Using Surface Modified Plates 4, 18, and 19 that Lack Extracellular Matrix Protein/Components and Feeder Cells

Passage 42 H1 human embryonic stem cells maintained on 1:30 MATRIGEL® coated plasticware in MEF conditioned media supplemented with 8 ng/ml of bFGF were lifted by LIBERASE™ enzymatic treatment and plated to surface modified 96 well format plates at a 1 to 2 dilution in MEF conditioned media supplemented with 8 ng/ml of bFGF. The cells were plated to modified surfaces 4, 18, or 19, or PRIMARIAT™. In order to determine the effect of Rho Kinase inhibition on binding to the modified surface we treated the cells with either 10 μM of the Rho Kinase inhibitor Y-27632, or 3 or 10 μM of the Rho Kinase inhibitor H1152-glycyl. Untreated cells served as controls. After 24 hours in culture the wells were aspirated, the cells were dried, and the wells were stained with Crystal violet.

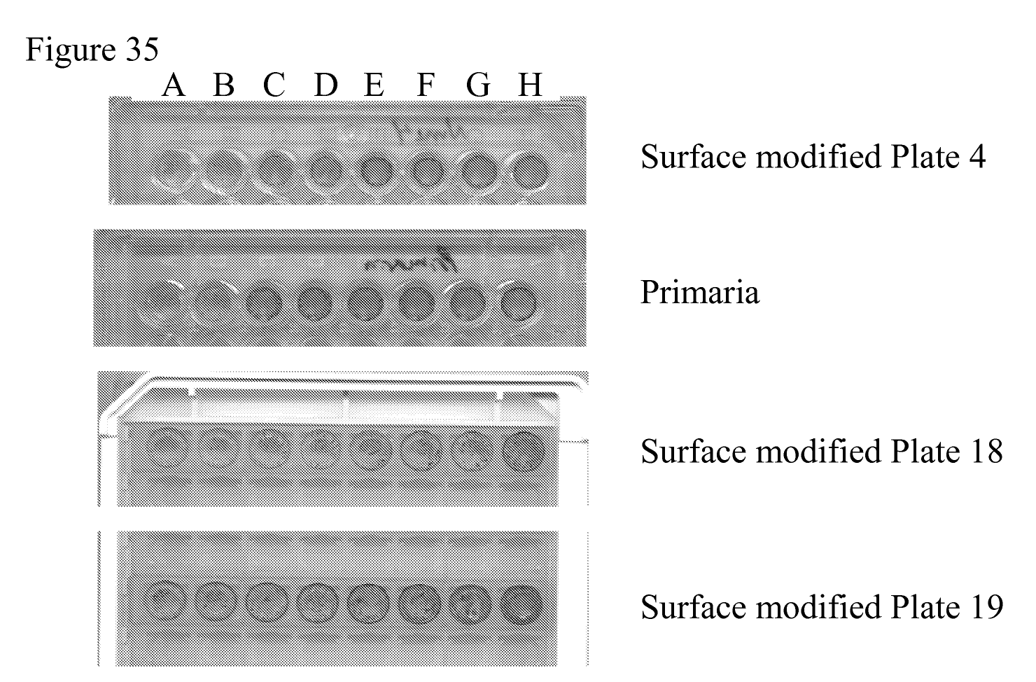

We observed that after 24 hours in culture, embryonic stem cell colonies had attached and spread when treated with Rho Kinase inhibitors on surface modified plates 4 and 19 and the PRIMARIAT™ plate, however the same effect was not observed on surface modified plate 18 (FIG. 35).

### Example 24

### Attachment, Cultivation and Maintenance of Human Embryonic Stem Cells Using Surface Modified Plates 30, 31, 32, 33, and 34 that Lack a Feeder-Cell Layer and an Adlayer

Passage 47 H1 human embryonic stem cells maintained on 1:30 MATRIGEL® coated plasticware in MEF conditioned media supplemented with 8 ng/ml of bFGF were lifted by TrypLE™ enzymatic treatment and plated to surface modified 96 well format plates at a 1 to 3 dilution in MEF conditioned media supplemented with 8 ng/ml of bFGF. The cells were plated to either surface modified plates 30, 31, 32, 33, or 34. In order to determine the effect of Rho Kinase inhibition on binding to the modified surface we treated the cells with 3 μM of the Rho Kinase inhibitor H1152-glycyl. Untreated cells served as controls. Additionally, cells were seeded to wells in the surface modified plate that were pre-treated with MATRIGEL®. 24 hours after plating the media was changed with fresh MEF conditioned media supplemented with 8 ng/ml of bFGF, and for cells seeded in the presence of the Rho Kinase inhibitor the media was supplemented with 3 μM H1152-glycyl. After 48 hours in culture the wells were aspirated, the cells were dried, and the wells stained with Crystal violet.

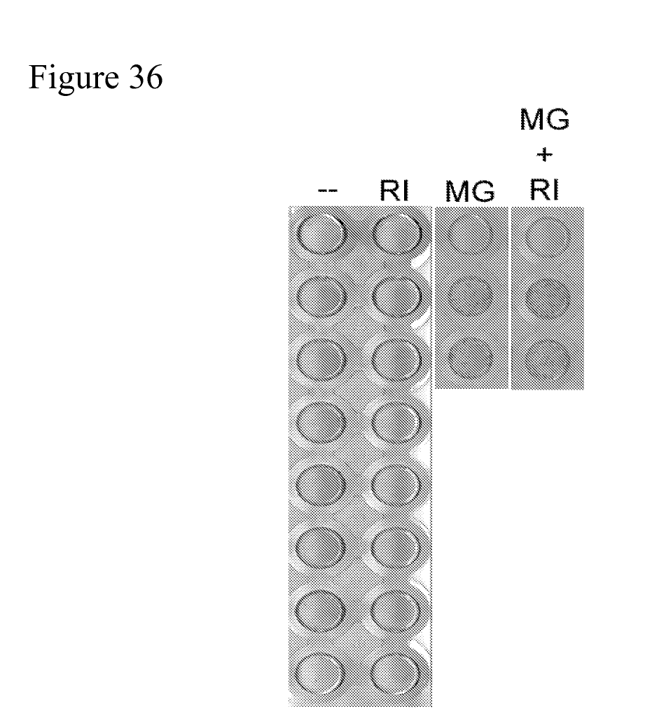

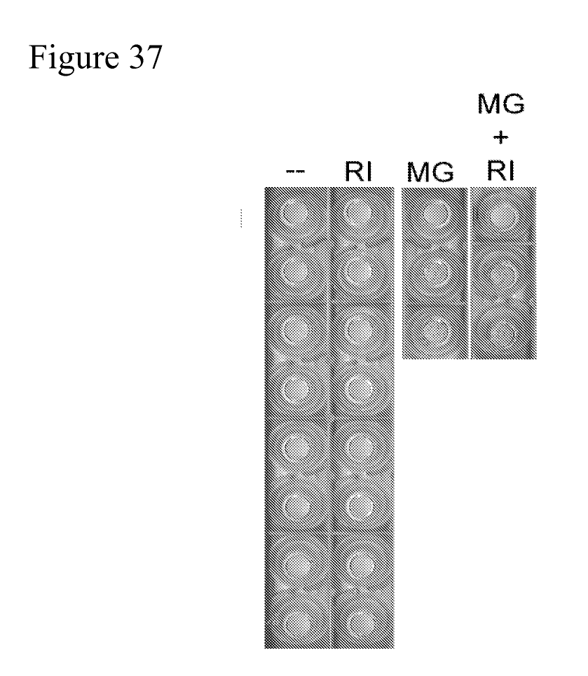

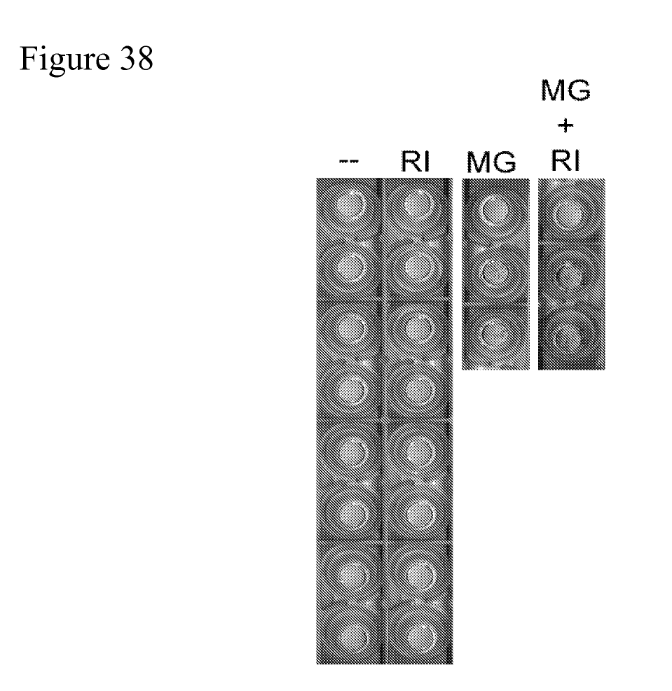

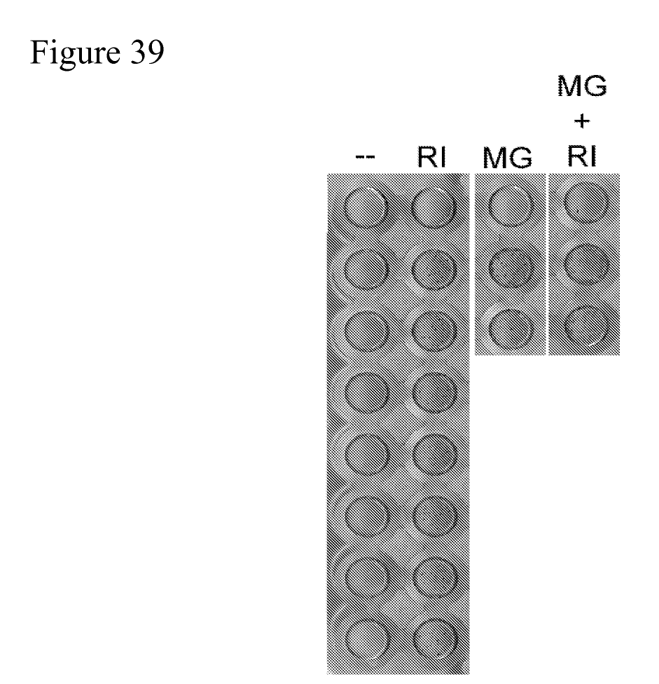

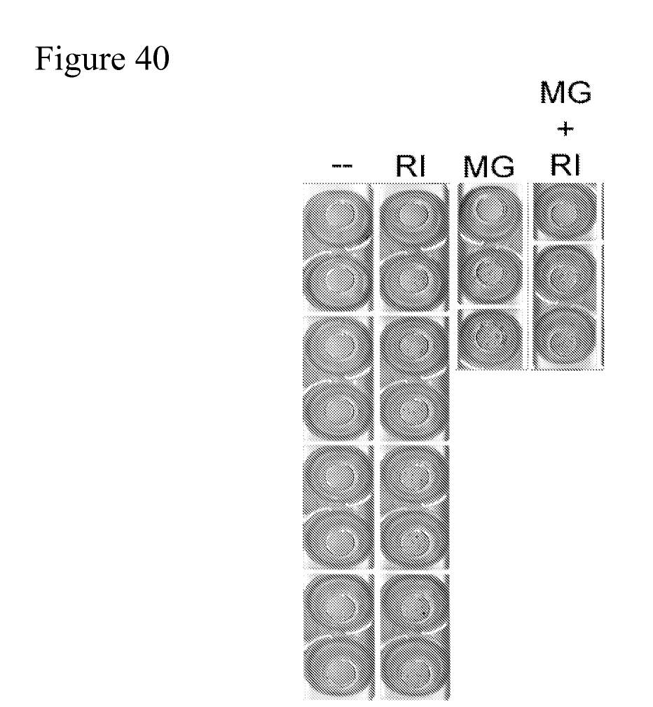

We observed that after 48 hours in culture, embryonic stem cell colonies had attached and spread when treated with Rho Kinase inhibitors on surface modified plates 33 and 34 (FIGS. 39 and 40 respectively), however the same effect was not observed on surface modified plates 30, 31 or 32 (FIGS. 36-40 respectively).

### Example 25

### Attachment, Cultivation and Maintenance of Human Embryonic Stem Cells Using Surface Modified Plates 22, 23, 24 or 29 that Lack a Feeder-Cell Layer and an Adlayer

Passage 46 H1 human embryonic stem cells maintained on 1:30 MATRIGEL® coated plasticware in MEF conditioned media supplemented with 8 ng/ml of bFGF were lifted by LIBERASE™ enzymatic treatment and plated to surface modified 60 mm dishes at a 1 to 3 dilution in MEF conditioned media supplemented with 8 ng/ml of bFGF. The cells were plated to surface modified plates 3, 4, 22, 23, 24 and 29. In order to determine the effect of Rho Kinase inhibition on binding to the modified surface we treated the cells with 3 μM of the Rho Kinase inhibitor H1152-glycyl to plate the cells. The media was changed with fresh MEF conditioned media supplemented with 8 ng/ml of bFGF and 1 μM of the Rho Kinase inhibitor H1152-glycyl 24 hours after plating the cells. Cells seeded to modified surface 3, 4 or MATRIGEL® coated plastic served as controls. The plates were observed by phase microscopy 24 and 48 hours after plating.

We observed that after 48 hours in culture, embryonic stem cell colonies had not attached to surface modified plates 22, 23, 24 or 29 plated with or without Rho Kinase inhibitor, while cells plated to surface modified plate 3 or 4 in the presence of Rho Kinase inhibitor did attach and spread.

### Example 26

### Further Surface Characterization of the Surface Modified Plates of the Present Invention

### Water Contact Angles

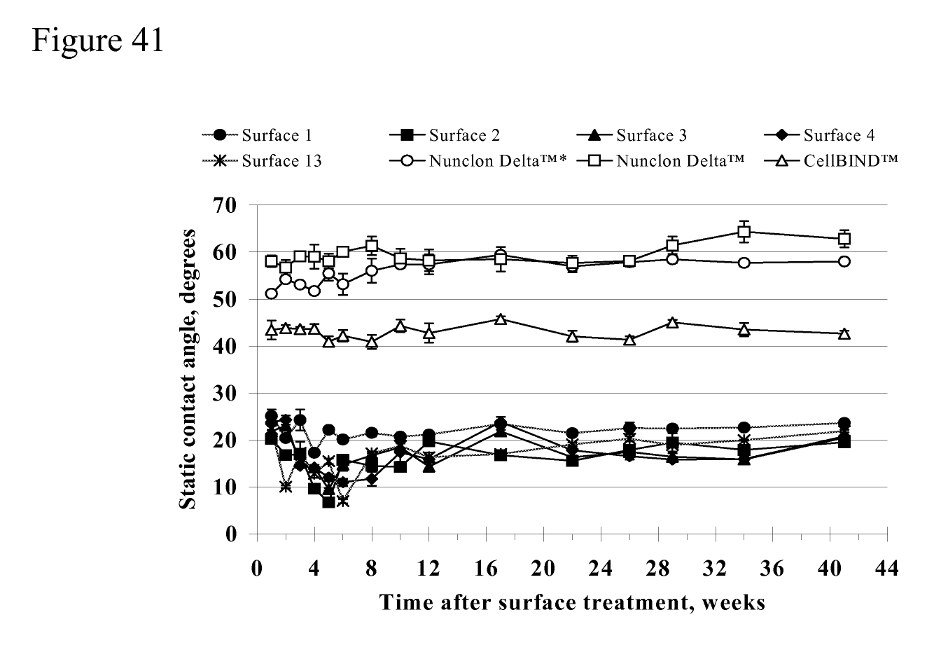

Surface modified plates 1-4 and 13 were individually packed in plastic bags, sterilized, and stored at room temperature throughout a 40-week test period. Contact angles were first measured one week after surface treatment and sterilization, and then again at the time points given in FIG. 41. All contact angle measurements were done as described in Example 17. Measurements on NUNCLON DELTA™ and CELLBIND™ surfaces was performed under the same experimental conditions as measurements on Surface 1-4 and 13, but the surface treatment and sterilization was done more than 12 weeks before the first measurement (NUNCLON DELTA™* was sterilized one week before the first measurement). FIG. 41 shows that surface modified plates 1-4 and 13 were of similar hydrophilicity and more hydrophilic (lower water contact angles) than NUNCLON DELTA™ and CELLBIND™ surfaces. The hydrophilicity of surface modified plates 1-4 and 13 was stable for at least 41 weeks after surface treatment and sterilization.

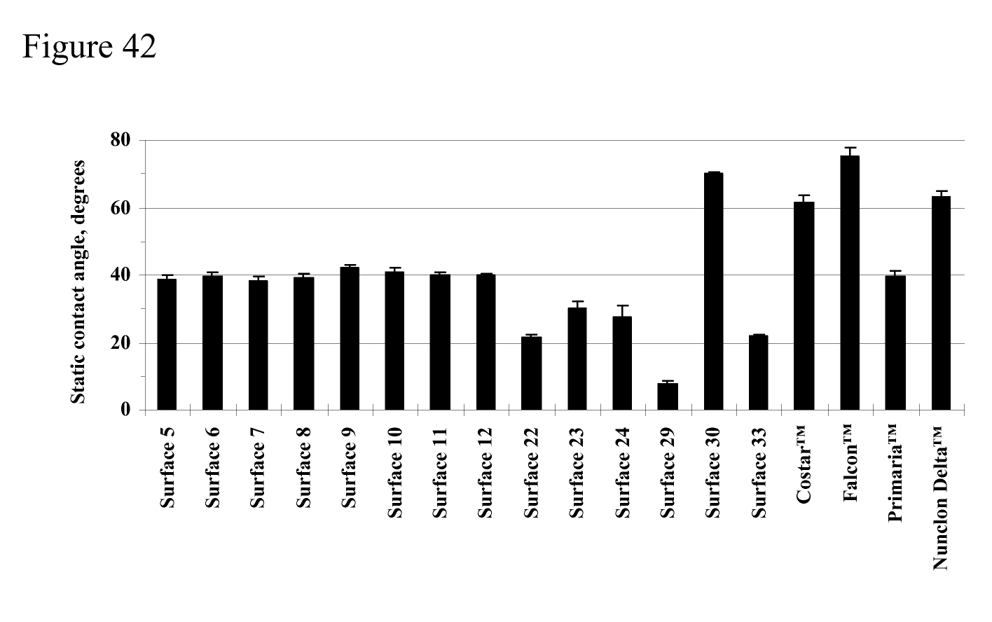

Contact angles were also measured on surface modified plates 5-12, 22-24, 29, 30 and 33, which were packed in plastic bags, sterilized as described in Example 16, and stored at room temperature for 9 weeks (except for surface modified plate 29 which was stored for 28 weeks). Surface modified plates 18, 19, 32 and 34 were in single-microwell format and could, therefore, not be used for measurements of contact angles. Surface modified plates 30 and 33 were in a microwell plate format, and contact angle measurements were performed on the backside of the plate and not inside wells. Contact angles were measured as described in Example 17 (for the highly hydrophilic surface modified plate 29, a smaller drop of 2.5 μl MilliQ water was applied), but triplicate samples were analyzed, with 7 drops being applied per sample. Measurements on plates with COSTAR™, FALCON™, PRIMARIA™ and NUNCLON DELTA™ surfaces was performed under the same experimental conditions, but the surface treatment and sterilization was done more than 12 weeks before the first measurement. FIG. 42 shows that surface modified plates 5-12 were more hydrophilic (lower water contact angles) than NUNCLON DELTA™, COSTAR™ and FALCON™ surfaces. The hydrophilicity of surfaces 5-12 was comparable to the hydrophilicity of the PRIMARIA™ surface, and higher than the hydrophilicity of surfaces 1-4 and 13 (shown in FIG. 41). The hydrophilicity of surface modified plates 22-24 and 33 was comparable to the hydrophilicity of surfaces 1-4 and 13 (shown in FIG. 41), whereas the hydrophilicity of surface 30 was comparable to the hydrophilicity of NUNCLON DELTA™, COSTAR™ and FALCON™ surfaces. Surface modified plate 29 was significantly more hydrophilic than the other surfaces analysed.

### Negative Charge Density

The density of negative charges on surface modified plates 5-12 (all 5-cm dish format), 18, 19, 30, 32, 33 and 34 (all microwell format), surface modified plates 22-24 and 29 (all 6-cm dish format), and CELLBIND™ surface (3-cm dish format), PRIMARIA™ surface (multidish-6 format) and NUNCLON DELTA™ surface (3-cm dish format) was determined Aqueous crystal violet solution (0.015% w/v) in excess was added to each format (0.34 ml/cm² for dish format and 0.13 ml/cm² for microwell format), and was incubated for 60 minutes at room temperature under gentle shaking (50 rpm). In order to remove crystal violet not bound to the surfaces, the dishes were washed three times with 3 ml MilliQ water for the dish formats and three times with 350 μl MilliQ water for microwell formats, and then dried overnight at 60° C. The crystal violet bound to the surface was desorbed by addition of 0.17 ml/cm² of 0.1 M HCl in EtOH solution (99%) and incubating the dishes for 2 minutes at room temperature under gentle shaking (50 rpm). Absorbance of the HCl:EtOH solution with desorbed crystal violet was measured at 590 nm using an EnVision 2100 microplate reader (Perkin Elmer; Waltham, Mass., USA). Absorbance values were corrected for background absorbance of HCl:EtOH solution. The negative charge density was measured on three dishes with surface modified plates 5-12, 22-24, 29, CELLBIND™, PRIMARIA™ and NUNCLON DELTA™, and absorbance measurements were performed in triplicate for each dish. For surface modified plates 18, 19, 30, 32, 33 and 34, one sample was tested with triplicate measurements.

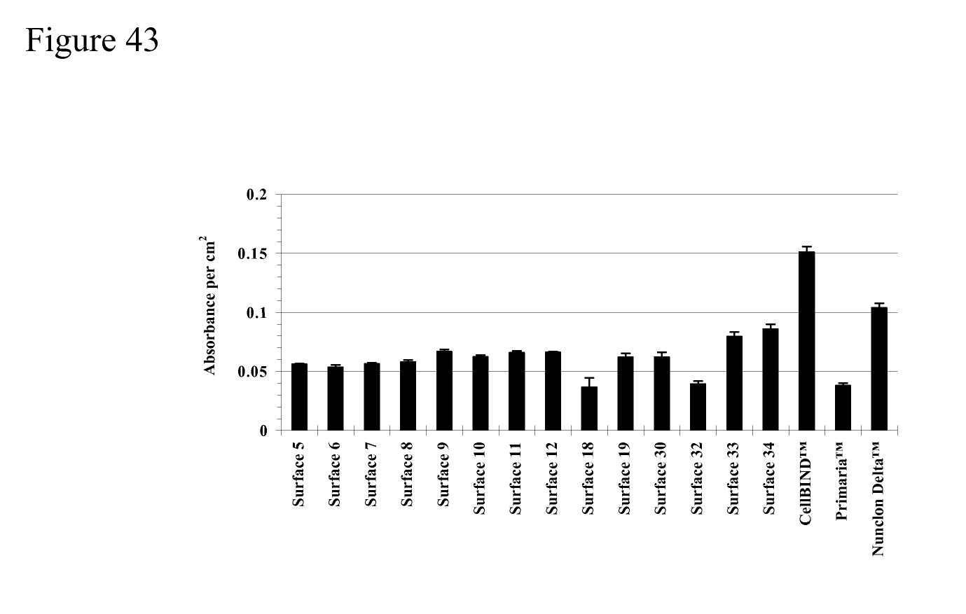

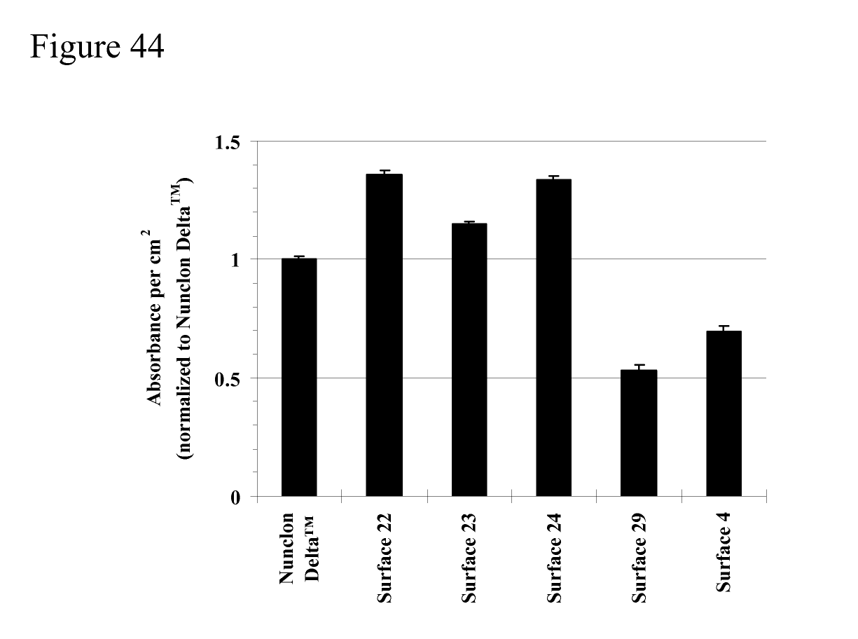

The negative charge densities of surface modified plates 5-12 were similar, and these surfaces had significantly lower negative charge densities than CELLBIND™ surface and NUNCLON DELTA™ surface, but significantly higher negative charge densities than the PRIMARIA™ surface (FIG. 43). The negative charge densities of surface modified plates 19, 33 and 34 were significantly higher than the negative charge densities of surface modified plates 18, 30 and 32, the latter being the respective non-treated surfaces in the same polymer material. The negative charge densities of surface modified plates 22-24 and 29 were normalized to the negative charge density of the NUNCLON DELTA™ surface, and FIG. 44 shows that surface modified plates 22-24 had higher negative charge densities than the NUNCLON DELTA™ surface, whereas the negative charge density for surface modified plate 29 was significantly lower than the negative charge density of the NUNCLON DELTA™ surface (and surface 4).

### X-Ray Photoelectron Spectroscopy (XPS)

Surface modified plates 5-12, 18, 19, 22-24, 29, 30, 31-34 were analyzed using XPS as described in Example 17. Surface elemental composition in units of atomic percent is shown in Table 12. All surfaces contained carbon, oxygen and nitrogen (hydrogen is not detected in XPS), except surface modified plates 31 and 32 (not plasma treated), which did not contain nitrogen. Surface modified plates 5-12 contained less oxygen than surface modified plates 1-4 and 13, but significantly more oxygen than COSTAR™, FALCON™ and NUNCLON DELTA™ surfaces (shown in Table 7). Surface modified plates 5-12, were prepared by microwave plasma treatment while surface modified plates 1-4 and 13 were produced by corona plasma treatment. Surface modified plates 19, 33 and 34, which were prepared by Corona plasma treatment, but injection molded from other polymers than polystyrene (which was used in the preparation of surface modified plates 1-4 and 13), contained oxygen levels comparable to those of surface modified plates 1-4 and 13. Surface modified plates 22-24 contained less oxygen than surface modified plates 1-4 and 13. Surface modified plate 29 contained oxygen at a level comparable to surface modified plates 1-4 and 13. Surface modified plates 5-12, 19, 33 and 34 contained less nitrogen than surface modified plates 1-4 and 13, but more nitrogen than COSTAR™, FALCON™ and NUNCLON DELTA™ surfaces (shown in Table 7). Surface modified plate 29 contained significantly more nitrogen than the other surfaces analysed, including the PRIMARIA™ surface.

C1s spectra peaks were curve fit (best chi-squared fit), in order to identify and quantify the bonding environments for carbon in the surface modified plates, by using peak widths and energy locations for species as found in the literature (Table 13). The concentrations are reported in units of atomic percent, which were obtained by multiplying the area percent by the atomic concentration. All plasma-treated ssurfaces, except surfaces 10, 22-24 and 29, were similar in terms of the carbon bonding environments. The proportion of carbon in C—[O]—C was significantly higher in Surfaces 19, 33 and 34 than in surfaces 5-12, 18, 30 and 32 and surfaces 1-4 and 13 (shown in Table 8). The proportion of carbon in O—[C═O]—O bonding environment was lower in surfaces 5-12, 19, 33 and 34 than in surfaces 1-4 and 13. The proportion of carbon in C*—C—O—C—C* was significantly higher in surfaces 5-9, 11, 12, 19, 33 and 34 than in surfaces 1-4 and 13, but comparable to NUNCLON DELTA™ and CELLBIND™ surfaces. The proportion of carbon in C—O—C or C—NH₃⁺ bonding environment (same energy location in spectra) was lower in surfaces 5-12, 19, 33 and 34 than in surfaces 1-4 and 13, but was higher than in COSTAR™, FALCON™, CELLBIND™ and PRIMARIA™ surfaces. The proportion of carbon in C—O—C*═O bonding environment was higher in surfaces 19, 33 and 34 than in surfaces 5-12, and comparable to the level in surfaces 1-4 and 13. The proportion of carbon in C═O bonding environment was higher in surfaces 5-12 than in surfaces 19, 33 and 34, but lower than in surfaces 1-4 and 13. The proportion of carbon in CO₃⁻ bonding environment was higher in surfaces 5-12 than in surfaces 19, 33 and 34, and comparable to the level in surfaces 1-4 and 13. Surfaces 22-24 were similar in terms of carbon bonding environments. The proportion of carbon in C—[O]—C, O—[C═O]—O, C—O—C or C—NH₃⁺, C—O—C*═O and C═O for surfaces 22-24 was significant lower than for surfaces 1-4 and 13. The proportion of carbon in CO₃⁻ and C*—C—O—C—C* bonding environments was higher for surfaces 22-24 than for surfaces 1-4 and 13. The carbon bonding environment of surface 29 was different from the carbon bonding environment of all other plasma-treated surfaces. The proportion of carbon in C—[O]—C was comparable to surfaces 1-4 and 13. The proportions of carbon in O—[C═O]—O, CO₃⁻ and C*—C—O—C—C* bonding environments were lower for surface 29 than for surfaces 1-4 and 13. The proportions of carbon in C—O—C or C—NH₃⁺, C—O—C*═O and C═O bonding environments were higher for surface 29 than for surfaces 1-4 and 13. The energy loss peak resulted from an aromatic Π→Π* transition, and is an indicator of surface aromaticity.

The O1s spectra peaks were almost Gaussian and could not be curve fit. Nls spectra peaks were curve fit (best chi-squared fit), in order to identify and quantify the bonding environments for nitrogen in the surfaces, by using peak widths and energy locations for species as found in the literature (Table 14). The concentrations are reported in units of atomic percent, which were obtained by multiplying the area percent by the atomic concentration. The proportion of nitrogen in —NH₃⁺ bonding environment in all surfaces, except surface 9, was lower than in surfaces 1-4 and 13. Nitrogen in —NH₂ bonding environment in surfaces 5-12, 19, 33 and 34 varied, but was higher than in surfaces 1-4 and 13. Nitrogen in —NO₂ bonding environment in surfaces 5-12, 19, 33 and 34 varied, but was lower than in surfaces 1-4 and 13. Nitrogen in —NO₃ bonding environment in surfaces 5-12, 19, 33 and 34 varied, but was higher than in surfaces 1-4 and 13. The nitrogen bonding environments of surfaces 22-24 and 29 were different from the other plasma-treated surfaces. The proportion of nitrogen in —NH₂ bonding environment in surfaces 22-24 and 29 varied, but was significantly higher than in surfaces 1-4 and 13. The proportion of nitrogen in —NO₂ bonding environment was lower in surfaces 22-24 and 29 than in surfaces 1-4 and 13. The proportion of nitrogen in O═C—N—C═O bonding environment was comparable in surfaces 22-24 and 29 and surfaces 1-4 and 13.

### Example 27

### Generation of Pluripotent Stem Cells from Amniotic Fluid-Derived Cells using the STEMGENT™ Human Transfection Factor Kit Without the use of Feeder Cells

100,000 amniotic fluid-derived cells isolated according to the methods disclosed in U.S. patent application Ser. No. 11/420,895 were plated in AMNIOMAX™ media in a 10 cm² well of a six well dish. The following day cells were transduced with lentivirus by changing the media to fresh AMNIOMAX™ media containing lentiviruses expressing SOX2, OCT4, LIN28, and NANOG, in a total volume of 2.5 ml. Lentivirus viral titer was determined by the manufacturer using p24 capsid antigen ELISA assay. Viral titers were as follows: SOX2-Lentivirus titer-68.80 ng/ml; OCT4-Lentivirus titer-6.64 ng/ml; LIN28-Lentivirus titer-68.80 ng/ml; and NANOG-Lentivirus titer-82.80 ng/ml.

On day 3, cells were split and plated to a surface coated with a 1:30 dilution of MATRIGEL® with MEFCM supplemented with 8 ng/ml of bFGF (MEFCM8) and 5 μM of the ROCK inhibitor Y27632 for passage zero (p0). Cells were plated and fed daily thereafter with fresh MEFCM8.

After one week in culture, cells were dispase passaged to fresh 1:30 MATRIGEL® coated plates (first passage=p1). 10 days later approximately 20 human embryonic stem cell like colonies were observed. The most dense “Embryonic stem cell like” colonies were manually selected and combined for passage. This culture was designated cell line AFD1. A single colony was manually selected for passage and designated AFD2.

### Example 28

### Culture of Pluripotent Stem Cells Derived from Somatic Cells on the Surface Modified Plates of the Present Invention

The pluripotent stem cell line derived from human Amniotic Fluid cells according to the methods outlined in the previous example was maintained in MEF conditioned media on NUNCLON DELTA™ plates treated with a 1:30 dilution of growth factor reduced MATRIGEL® prior to study. Cells were dissociated from the surface for passage by 1 mg/ml dispase dissociation. AFD1 cells were then seeded onto untreated wells of surface modified plate 4 (6-well format). In parallel, AFD1 cells were also plated onto NUNCLON DELTA™ plates treated with 1:30 dilution of growth factor reduced MATRIGEL® to provide as positive controls. In all treatments cells were maintained in MEF conditioned media.

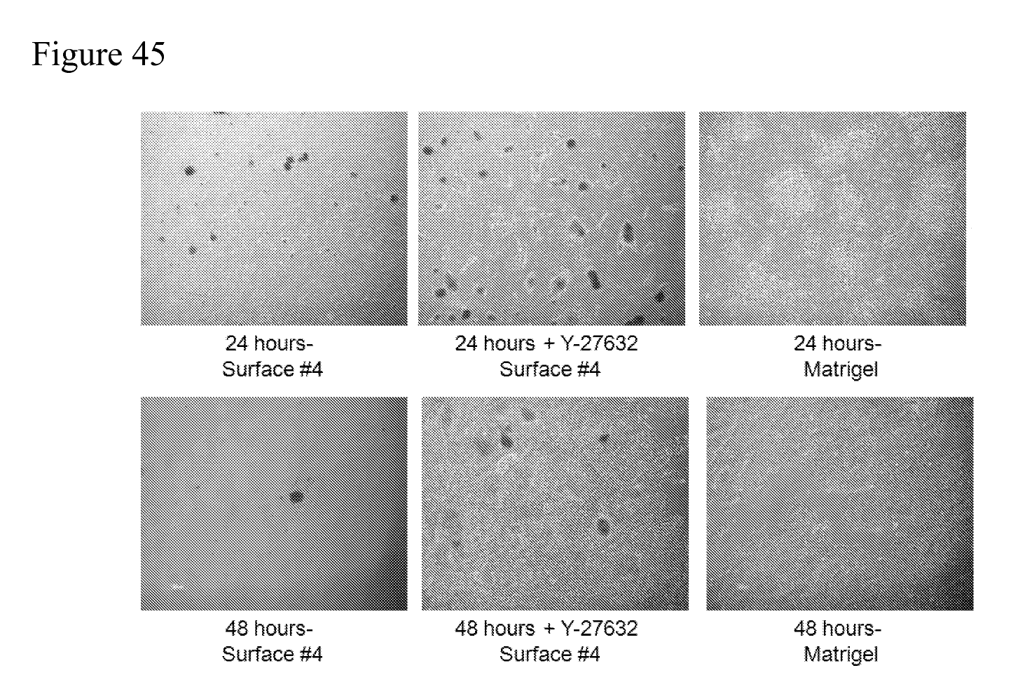

The pluripotent stem cells seeded onto surface modified plate 4 did attach, however the attachment efficiency was much lower than on control plates (FIG. 45). The attachment efficiency was greatly increased by the addition of a Rho Kinase inhibitor to the media. Adding either Y-27632 at a 10 μM concentration or H1152-glycyl at a 3 μM concentration for the first 24 hours in culture significantly increased plating efficiency of the cells to a rate similar observed with control coated plates (FIG. 45). Cells were thereafter maintained in a constant 10 μM Y-27632 concentration or a reduced 1 μM concentration of H1152-glycyl, respectively, with daily media change.

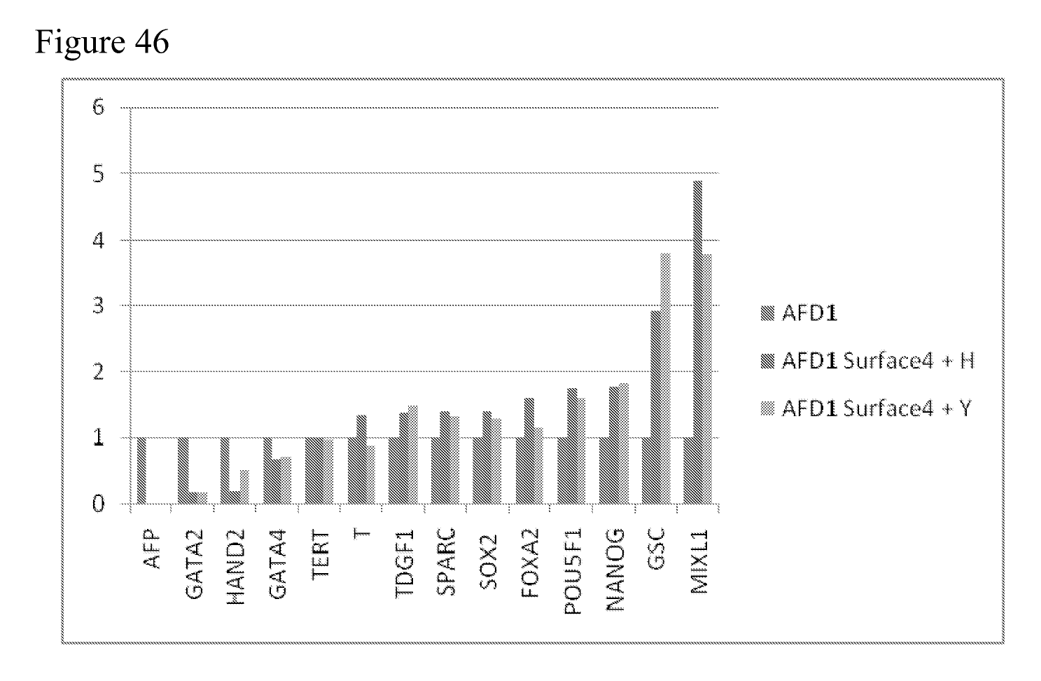

Cells were passaged twice on surface modified plate 4 in the presence of either Y-27632 or H1152-glycyl and were tested for the expression of genes associated with pluripotency by qRT-PCR (FIG. 46). Expression of genes associated with and required for pluripotency (OCT4, NANOG, SOX2, TERT, and CRIPTO/TDGF) was maintained in AFD1 cells grown on modified surface 4 versus cells grown on control plates. We also observed that the relative expression of several genes (AFP, HAND2, and GATA2) associated with spontaneous differentiation to extra-embryonic ectoderm or a trophoblast fate was decreased significantly. This may have been due to increased expression of NANOG, which inhibits differentiation by suppressing expression of extra-embryonic ectoderm or trophoblast associated genes. Additionally, this decrease in expression of extra-embryonic ectoderm or trophoblast associated genes could occur through differential adhesion and proliferation of cells more pluripotent and less differentiated, on modified surface 4 versus control plates.

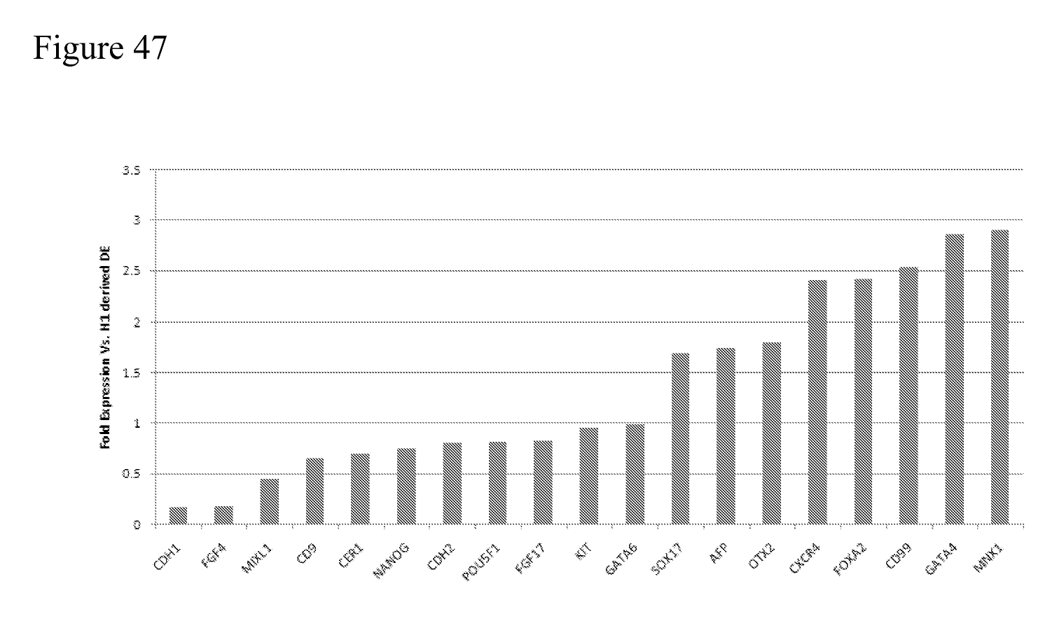

The pluripotency of cells grown on modified surface 4 was also confirmed by testing their capacity to differentiate to definitive endoderm. We observed that cells differentiated to DE after growing several passages on modified surface 4 in MEF Conditioned Culture Media supplemented with Rho Kinase Inhibitor showed robust expression of genes associated with DE differentiation, comparable to expression levels and patterns observed with H1 human embryonic stem cell differentiation to DE (FIG. 47).

### Example 29

### Human Embryonic Stem Cells Cultured in the Absence of a Feeder-Cell Layer or an Adlayer, with a Rho Kinase Inhibitor Retain Pluripotency After Being Stored in Liquid Nitrogen, Thawed, and Passaged

H1 human embryonic stem cells grown on modified surface 4 for 11 passages (described in FIG. 10) as were frozen, stored in liquid nitrogen, thawed, passaged twice, and then implanted in mice to test teratoma formation and retention of pluripotency. In brief, we froze H1 human embryonic stem cells after 11 passages on modified surface 4 in a solution containing 10% DMSO, 30% FBS, and %60 hESC culture media. The cells were thawed from liquid nitrogen storage onto a MEF feeder layer in hESC culture media, and then serially passaged twice onto NUNCLON DELTA™ plates coated with a 1:30 dilution of growth factor reduced MATRIGEL®. After two passages on MATRIGEL® the cells were lifted with TrypLE™ and eight million cells were implanted under the kidney capsule of three SCID beige mice. After eight weeks we observed tumors in all three mice.

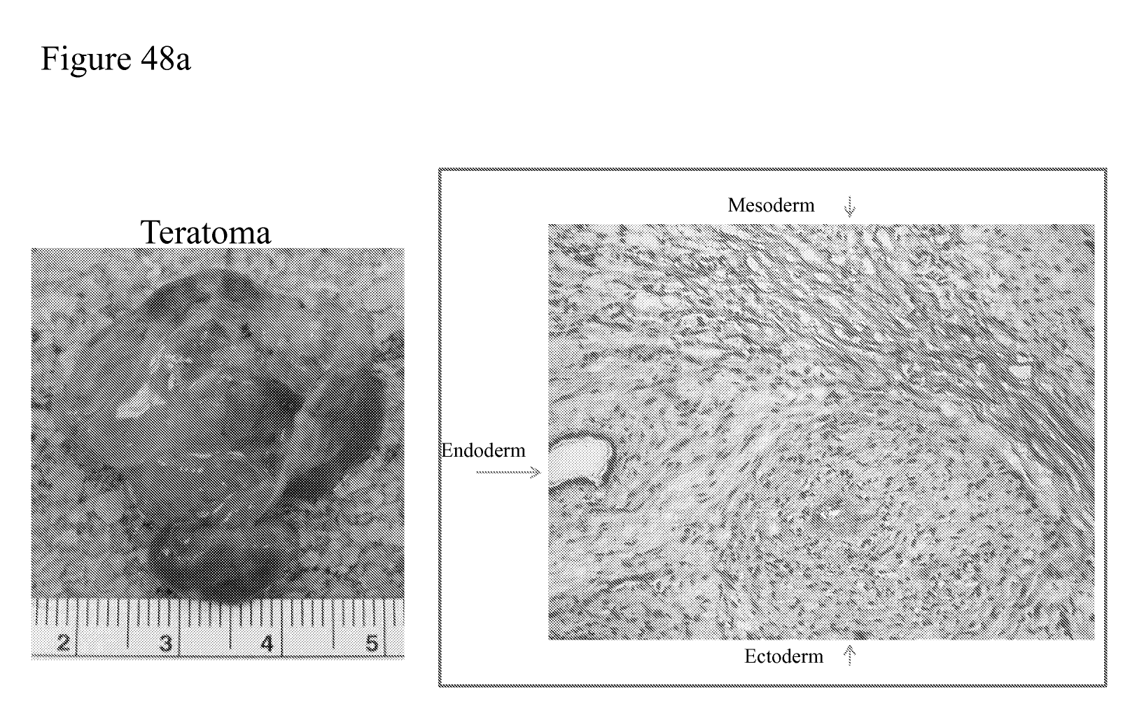

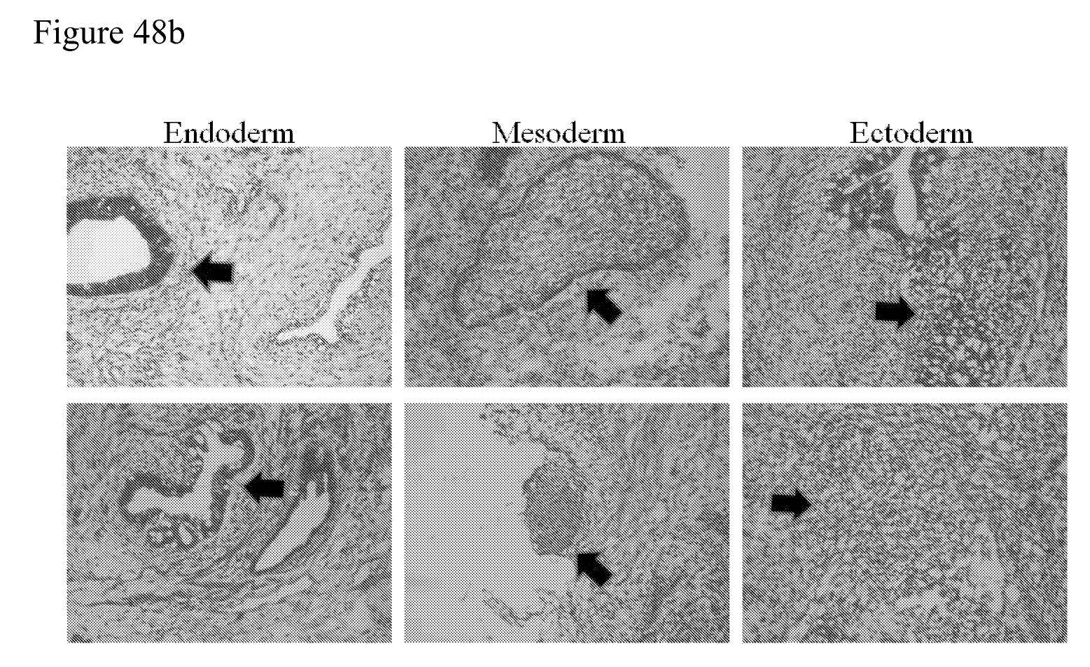

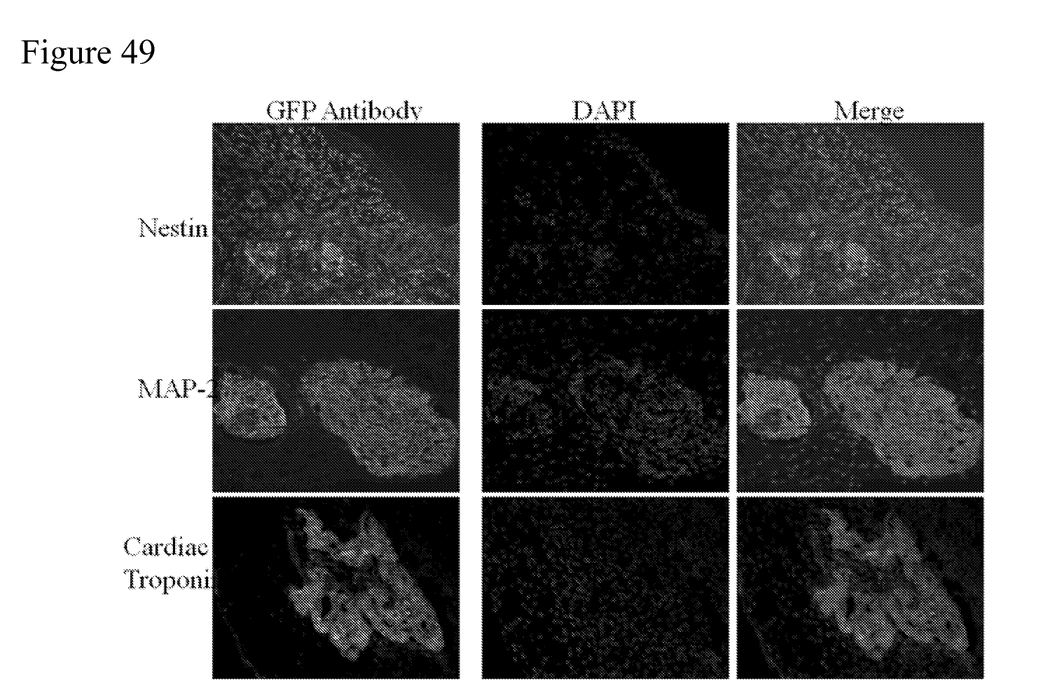

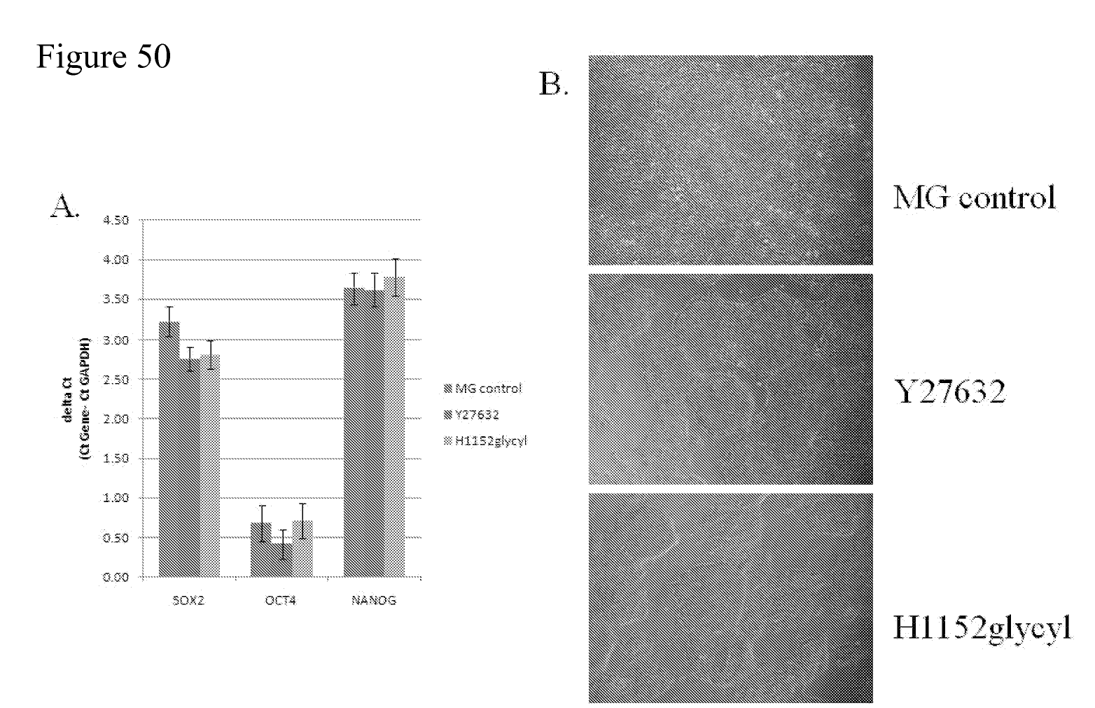

We euthanized the mice and removed the kidney and associated tumor for analysis. By H&E (FIGS. 48a and b) and immunofluorescent staining (FIG. 49) of sections we observed cells and tissues representing each of the three germ layers (endoderm, mesoderm, and ectoderm). The presence of epithelial tissues (endoderm) was relatively straightforward to detect by H&E, and we confirmed mesoderm and ectoderm formation by immunofluorescent staining. Using antibodies to Nestin and Microtubule-associated protein 2 (MAP2), proteins expressed by neural tissues (ectoderm), and an antibody to Cardiac Troponin I (mesoderm), we confirmed the presence of tissues representing all three germ layers. These results indicate that after 11 passages on modified surface 4, liquid nitrogen storage, thaw and passage, H1 human embryonic stem cells retained pluripotency and were competent to form tissues from all three germ layers.

### Example 30

### Human Embryonic Stem Cells Cultured 10 Passages in the Absence of a Feeder-Cell Layer or an Adlayer on surface modified plate 4 with the Rho Kinase Inhibitor Y27632 or H1152-glycyl Retain Pluripotency

H1 human embryonic stem cells grown on surface modified plate 4 for 10 passages with either Rho Kinase Inhibitor Y27632 or H1152-glycyl demonstrated nearly identical expression levels of the pluripotency genes SOX2, OCT4, and NANOG over 10 passages as measured by qRT-PCR and the cultures were morphologically indistinguishable from control cultures grown on MATRIGEL®. The expression of SOX, OCT4, and NANOG were measured by qRT-PCR at each passages over 10 passages on surface modified plate 4 for 10 passages with either Rho Kinase Inhibitor Y27632 or H1152-glycyl. The cycle of detection (Ct) was determined as was the Ct for a standard housekeeping gene. We subtracted the Ct GAPDH from the Ct for SOX2, OCT4, and NANOG and calculated the mean and standard error of the mean for expression levels of the genes over 10 passages.

We observed that gene expression was maintained constant over 10 passages with minimal variability (standard error of the mean) in H1 human embryonic stem cells maintained on surface modified plate 4 with either Y-27632 or H1152-glycyl. Furthermore H1 human embryonic stem cells passaged 10 times on modified surface 4 and plated on MATRIGEL® retained morphological features of cells passaged on MATRIGEL®, and were morphologically indistinguishable. We conclude that use of the either Rho Kinase Inhibitor Y27632 or H1152-glycyl maintains pluripotency in pluripotent stem cells cultured on modified surface 4.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

### Tables

TABLE 1
Expression of Pluripotency Markers in Cells of the Human Embryonic Stem Cell Line
H1 at Passage 50, Cultured on the Surface Modified Plates of the Present Invention

| Culture | Marker | | | | | | | | | |
|---|---|---|---|---|---|---|---|---|---|---|
| Condition | CFC1 | GATA2 | GJA7 | NANOG | OCT4 | SOX2 | SOX7 | TERT | TUBB3 | ZIC1 |
| Costar | 1.0 | 1.0 | 1.0 | 1.0 | 1.0 | 1.0 | 1.0 | 1.0 | 1.0 | 1.0 |
| 1:30 | | | | | | | | | | |
| MATRIGEL | | | | | | | | | | |
| Surface | 0.3 | 0.5 | 0.8 | 0.8 | 0.4 | 0.8 | 2.1 | 0.6 | 3.2 | 1.5 |
| Modified | | | | | | | | | | |
| Plate 2 | | | | | | | | | | |
| Gelatin | | | | | | | | | | |
| Surface | 1.0 | 0.4 | 0.9 | 0.9 | 0.7 | 1.1 | 1.9 | 0.7 | 2.8 | 1.5 |
| Modified | | | | | | | | | | |
| Plate 2 | | | | | | | | | | |
| No Coating | | | | | | | | | | |
| Surface | 0.3 | 0.5 | 0.7 | 0.7 | 0.3 | 0.6 | 0.8 | 1.0 | 3.1 | |
| Modified | | | | | | | | | | |
| Plate 2 | | | | | | | | | | |
| No Coating | | | | | | | | | | |
| Surface | 0.5 | 0.6 | 0.7 | 0.8 | 0.5 | 0.8 | 1.4 | 0.9 | 3.0 | 3.0 |
| Modified | | | | | | | | | | |
| Plate 3 | | | | | | | | | | |
| Gelatin | | | | | | | | | | |
| Surface | 0.3 | 0.5 | 0.7 | 0.8 | 0.5 | 0.6 | 1.9 | 1.0 | 3.9 | 0.9 |
| Modified | | | | | | | | | | |
| Plate 3 | | | | | | | | | | |
| No Coating | | | | | | | | | | |
| Surface | 0.3 | 0.7 | 0.8 | 1.3 | 0.5 | 0.8 | 1.4 | 1.2 | | 1.0 |
| Modified | | | | | | | | | | |
| Plate 3 | | | | | | | | | | |
| No Coating | | | | | | | | | | |
| Surface | 0.4 | 0.4 | 0.7 | 1.0 | 0.5 | 0.9 | 1.2 | 1.0 | 3.2 | 2.9 |
| Modified | | | | | | | | | | |
| Plate 4 | | | | | | | | | | |
| Gelatin | | | | | | | | | | |
| Surface | 0.5 | 0.6 | 1.1 | 1.2 | 0.9 | 1.4 | 2.3 | 1.3 | 4.1 | 1.5 |
| Modified | | | | | | | | | | |
| Plate 4 | | | | | | | | | | |
| No Coating | | | | | | | | | | |
| Surface | 1.0 | 0.3 | 1.8 | 1.0 | 0.8 | 1.3 | 0.8 | 0.9 | 2.1 | 1.7 |
| Modified | | | | | | | | | | |
| Plate 4 | | | | | | | | | | |
| No Coating | | | | | | | | | | |

TABLE 2
Expression of Pluripotency Markers in Cells of the Human Embryonic Stem Cell Line
H1 at Passage 53 Cultured on the Surface Modified Plates of the Present Invention

| Culture | MARKER | | | | | | | | | |
|---|---|---|---|---|---|---|---|---|---|---|
| Condition | CFC1 | GATA2 | GJA7 | MIXL1 | NANOG | OCT4 | SOX2 | SOX7 | TERT | TUBB3 |
| Costar | 1.0 | 1.0 | 1.0 | 1.0 | 1.0 | 1.0 | 1.0 | 1.0 | 1.0 | 1.0 |
| 1:30 | | | | | | | | | | |
| MATRIGEL | | | | | | | | | | |
| Surface | 6.22 | 33.25 | 1.42 | 3.98 | 0.13 | 0.46 | 0.42 | 2.81 | 0.50 | 3.69 |
| Modified | | | | | | | | | | |
| Plate 2 | | | | | | | | | | |
| Surface | 15.80 | 16.78 | 1.11 | 20.64 | 0.75 | 1.90 | 1.25 | 1.89 | 1.83 | 1.37 |
| Modified | | | | | | | | | | |
| Plate 3 | | | | | | | | | | |
| Surface | 11.41 | 17.58 | 8.61 | 14.06 | 4.46 | 20.22 | 6.39 | 1.91 | 14.42 | 31.53 |
| Modified | | | | | | | | | | |
| Plate 4 | | | | | | | | | | |

TABLE 3
Expression of Markers Characteristic of the Definitive Endoderm
Lineage in Cells of the Human Embryonic Stem Cell Line H1 at
Passage 51 (p2) and Passage 53 (p4) Cultured on the Surface Modified
Plates of the Present Invention, Treated with Activin A

| | p2 | p4 |
|---|---|---|
| Surface #4 | 45.5 | 74.8 |

% Expression of the surface marker CXCR4 following differentiation of H1 human ES cells to definitive endoderm

TABLE 4
Percent confluence (acquisition area occupied by objects) and total human H9
embryonic stem cell colonies greater than 50K sq. microns in the acquisition
area after one passage on the Surface Modified Plates of the Present Invention

| | | | | | PERCENT |
|---|---|---|---|---|---|
| | | | | | ACQUISITION |
| | | PERCENT | | | AREA OCCUPIED |
| | | ACQUISITION | ACQUISITION | TOTAL | BY hES |
| NUNC | | AREA OCCUPIED | AREA OCCUPIED | OBJECTS IN | COLONIES >50K |
| MODIFIED | RI | BY OBJECTS | BY OBJECTS | ACQUISITION | sq. microns |
| SURFACE | EXPOSURE | [%] | [sq. microns] | AREA | [%] |
| 2 | 0 | 4.4 | 6.83E+06 | 942 | 3.6 |
| 2 | 1 | 0.4 | 6.89E+05 | 348 | 0.0 |
| 2 | 4 | 9.9 | 1.55E+07 | 2,433 | 8.1 |
| 2 | 10 | 79.4 | 1.24E+08 | 1,146 | 79.2 |
| 3 | 0 | 2.2 | 3.39E+06 | 1,601 | 1.2 |
| 3 | 1 | 4.1 | 6.45E+06 | 1,347 | 3.0 |
| 3 | 4 | 37.5 | 5.85E+07 | 3,230 | 33.9 |
| 3 | 10 | 69.4 | 1.08E+08 | 1,587 | 67.8 |
| 4 | 0 | 15.8 | 2.47E+07 | 1,131 | 26.0 |
| 4 | 1 | 6.9 | 1.07E+07 | 1,507 | 5.7 |
| 4 | 4 | 39.1 | 6.10E+07 | 2,180 | 36.8 |
| 4 | 10 | 92.1 | 1.44E+08 | 449 | 91.8 |
| 13 | 0 | 1.4 | 2.11E+06 | 254 | 0.9 |
| 13 | 1 | 0.2 | 2.97E+05 | 301 | 0.0 |
| 13 | 4 | 10.5 | 1.64E+07 | 6,200 | 5.2 |
| 13 | 10 | 69.4 | 1.08E+08 | 1,587 | 67.8 |

| | | ACQUISITION | | | |
|---|---|---|---|---|---|
| | | AREA OCCUPIED | TOTAL hES | | |
| | | BY hES | COLONIES >50K | | |
| NUNC | | COLONIES >50K | sq. microns IN | ACQUISITION | 10X FIELD |
| MODIFIED | RI | sq. microns | ACQUISITION | AREA | AREA |
| SURFACE | EXPOSURE | [sq. microns] | AREA | [sq. micron] | [sq micron] |
| 2 | 0 | 5.61E+06 | 22 | 1.56E+08 | 6.02E+05 |
| 2 | 1 | 5.33E+04 | 1 | 1.56E+08 | 6.02E+05 |
| 2 | 4 | 1.26E+07 | 47 | 1.56E+08 | 6.02E+05 |
| 2 | 10 | 1.24E+08 | 262 | 1.56E+08 | 6.02E+05 |
| 3 | 0 | 1.82E+06 | 10 | 1.56E+08 | 6.02E+05 |
| 3 | 1 | 4.74E+06 | 21 | 1.56E+08 | 6.02E+05 |
| 3 | 4 | 5.29E+07 | 151 | 1.56E+08 | 6.02E+05 |
| 3 | 10 | 1.06E+08 | 270 | 1.56E+08 | 6.02E+05 |
| 4 | 0 | 4.05E+07 | 96 | 1.56E+08 | 6.02E+05 |
| 4 | 1 | 8.96E+06 | 29 | 1.56E+08 | 6.02E+05 |
| 4 | 4 | 5.74E+07 | 149 | 1.56E+08 | 6.02E+05 |
| 4 | 10 | 1.43E+08 | 264 | 1.56E+08 | 6.02E+05 |
| 13 | 0 | 1.45E+06 | 5 | 1.56E+08 | 6.02E+05 |
| 13 | 1 | 0.00E+00 | 0 | 1.56E+08 | 6.02E+05 |
| 13 | 4 | 8.17E+06 | 30 | 1.56E+08 | 6.02E+05 |
| 13 | 10 | 1.06E+08 | 270 | 1.56E+08 | 6.02E+05 |

μM concentration of Y-27632 for 96 hour
RI exposure: culture

TABLE 5
Expression of Markers Characteristic of the Definitive Endoderm
Lineage in Cells of the Human Embryonic Stem Cell Line H1
at Passage 51 Cultured on the Surface Modified Plates of
the Present Invention, Treated with Activin A

| | p8 | p10-11 |
|---|---|---|
| Surface #4 (1s) | 62.7 | 55.5 |
| Surface #4 (3s) | 68.4 | 41.4 |
| Surface #3 (5s) | N/A | 55.6 |
| Surface #3 (7s) | 62.5 | 52 |

% Expression of the surface marker CXCR4 following differentiation of H1 human ES cells to definitive endoderm

TABLE 6
Preparation of the Surface Modified Plates of the Present Invention
Preparation of Surface Modified Plates

| | | | | | |
|---|---|---|---|---|---|
| | | Power | Time | P | |
| Surface | Polymer | (W) | (s) | (mbar) | Gas |

Corona Plasma

| | | | | | |
|---|---|---|---|---|---|
| 1 | Polystyrene | 2000 | 5 | 1E−02 | Air |
| 13 | Polystyrene | 2000 | 10 | 1E−02 | Air |
| 2 | Polystyrene | 2000 | 15 | 1E−02 | Air |
| 3 | Polystyrene | 2000 | 30 | 1E−02 | Air |
| 4 | Polystyrene | 2000 | 60 | 1E−02 | Air |
| 19 | Cyclic olefin copolymer | 2000 | 60 | 1E−02 | Air |
| 33 | Polycarbonate/polystyrene | 2000 | 60 | 1E−02 | Air |
| 34 | Polycarbonate | 2000 | 60 | 1E−02 | Air |

Microwave Plasma

| | | | | | |
|---|---|---|---|---|---|
| 5 | Polystyrene | 600 | 6 | 0.3-0.4 | Air |
| 6 | Polystyrene | 600 | 12 | 0.3-0.4 | Air |
| 7 | Polystyrene | 600 | 18 | 0.3-0.4 | Air |
| 8 | Polystyrene | 600 | 24 | 0.3-0.4 | Air |
| 9 | Polystyrene | 600 | 6 | 0.3-0.4 | Oxygen |
| 10 | Polystyrene | 600 | 12 | 0.3-0.4 | Oxygen |
| 11 | Polystyrene | 600 | 18 | 0.3-0.4 | Oxygen |
| 12 | Polystyrene | 600 | 24 | 0.3-0.4 | Oxygen |
| 14* | Polystyrene | 500 | 60 | 0.4-0.5 | Air |
| 15 | Polystyrene | 500 | 60 | 0.4-0.5 | Air |

*Not sterilized by gamma irradiation

TABLE 7
Surface Elemental Composition of Surface Modified Plates as Determined by XPS
Surface Elemental Composition of Surface Modified Plates as Determined by XPS

| | % Carbon | | | % Oxygen | | | % Nitrogen | | |
|---|---|---|---|---|---|---|---|---|---|
| Surface | 1 | 2 | Mean ± SD | 1 | 2 | Mean ± SD | 1 | 2 | Mean ± SD |
| Surface 1 | 74.2 | 76.1 | 75.1 ± 0.3 | 24.6 | 22.6 | 23.6 ± 1.4 | 1.3 | 1.3 | 1.3 ± 0.0 |
| Surface 2 | 69.0 | 72.0 | 70.5 ± 2.1 | 29.3 | 26.5 | 27.9 ± 2.0 | 1.8 | 1.6 | 1.7 ± 0.1 |
| Surface 3 | 68.5 | 70.2 | 69.4 ± 1.2 | 29.3 | 28.0 | 28.7 ± 0.9 | 2.2 | 1.8 | 2.0 ± 0.3 |
| Surface 4 | 69.2 | 70.5 | 69.9 ± 0.9 | 28.8 | 27.5 | 28.2 ± 0.9 | 2.1 | 2.0 | 2.1 ± 0.1 |
| Surface 13 * | 69.9 | 73.5 | 71.7 ± 2.5 | 27.9 | 24.9 | 26.4 ± 2.1 | 1.9 | 1.6 | 1.8 ± 0.2 |
| Surface 14 | 82.8 | 82.5 | 82.7 ± 0.2 | 15.6 | 15.6 | 15.6 ± 0.0 | 1.6 | 1.8 | 1.7 ± 0.1 |
| Surface 15 | 78.6 | 78.7 | 78.7 ± 0.1 | 20.0 | 20.0 | 20.0 ± 0.0 | 1.4 | 1.3 | 1.4 ± 0.1 |
| Nunclon | 84.8 | 84.6 | 84.7 ± 0.1 | 14.7 | 14.7 | 14.7 ± 0.0 | 0.5 | 0.6 | 0.6 ± 0.1 |
| Delta ™ | | | | | | | | | |
| CellBIND ™ | 72.3 | 72.2 | 72.2 ± 0.1 | 26.7 | 26.9 | 26.8 ± 0.1 | 1.0 | 0.9 | 1.0 ± 0.1 |
| Falcon ™ | 95.2 | 94.7 | 94.9 ± 0.4 | 4.7 | 5.1 | 4.9 ± 0.3 | 0.1 | 0.3 | 0.2 ± 0.1 |
| Costar ™ | 85.8 | 85.4 | 85.6 ± 0.3 | 13.8 | 14.4 | 14.1 ± 0.4 | 0.4 | 0.2 | 0.3 ± 0.1 |
| Primaria ™ | 77.7 | 76.5 | 77.0 ± 0.8 | 12.7 | 12.9 | 12.8 ± 0.1 | 9.7 | 10.6 | 10.2 ± 0.6 |

Measurements on two samples and mean ± standard deviation (SD) is given in units of atomic percent
* Other elements were detected at a concentration of 0.4%

TABLE 8
Carbon Bonding Environment by C1s Spectra Curve Fitting
Carbon Bonding Environment by C1s Spectra Curve Fitting

| | Functional groups and C1s binding energies in eV | | | | |
|---|---|---|---|---|---|
| | | | C—O—C, | | |
| | C—C | C*—C—O—C—C* | C—NH₃⁺ | C—[O]—C | C═O |
| Surface | (284.6 eV) | (285.2 eV) | (286.1 eV) | (287.0 eV) | (287.9 eV) |
| Surface 1 | 34.5 ± 0.6 | 11.4 ± 0.7 | 14.1 ± 0.7 | 4.2 ± 0.5 | 4.5 ± 0.1 |
| Surface 2 | 31.7 ± 1.3 | 7.9 ± 1.5 | 13.4 ± 0.8 | 5.4 ± 0.3 | 4.3 ± 0.6 |
| Surface 3 | 31.5 ± 1.5 | 7.4 ± 2.0 | 12.7 ± 2.1 | 4.8 ± 0.1 | 4.8 ± 0.1 |
| Surface 4 | 31.6 ± 0.5 | 6.5 ± 2.7 | 14.1 ± 1.3 | 4.1 ± 0.0 | 4.7 ± 0.5 |
| Surface 13 | 33.6 ± 0.6 | 6.7 ± 3.2 | 13.2 ± 0.5 | 4.9 ± 0.4 | 4.9 ± 0.3 |
| Surface 14 | 49.7 ± 2.6 | 17.2 ± 3.5 | 8.1 ± 1.7 | 3.2 ± 0.3 | 2.2 ± 0.6 |
| Surface 15 | 46.7 ± 0.1 | 20.0 ± 0.0 | 11.2 ± 4.0 | 4.0 ± 0.9 | 2.9* |
| Nunclon | 36.2 ± 1.3 | 28.2 ± 1.3 | 7.9 ± 0.5 | 3.9 ± 0.6 | 3.5 ± 0.1 |
| Delta ™ | | | | | |
| CellBIND ™ | 27.6 ± 1.1 | 19.2 ± 0.6 | 5.7 ± 0.0 | 6.5 ± 2.0 | 5.7 ± 0.0 |
| Falcon ™ | 76.2 ± 3.2 | 12.0 ± 3.5 | 5.7 ± 1.0 | 0.1 ± 0.2 | 0.0 ± 0.0 |
| Costar ™ | 60.8 ± 6.0 | 12.5 ± 5.4 | 7.0 ± 2.0 | 2.1 ± 0.4 | 0.5 ± 0.3 |
| Primaria ™ | 50.1 ± 1.8 | 11.1 ± 0.2 | 1.9 ± 2.3 | 4.8 ± 0.2 | 3.1 ± 0.1 |

| | Functional groups and C1s binding energies in eV | | | |
|---|---|---|---|---|
| | | | | Energy |
| | C—O—C*═O | CO₃⁻ | O—[C═O]—O | loss peak |
| Surface | (288.9 eV) | (289.8 eV) | (291.0 eV) | (292 eV) |
| Surface 1 | 1.7 ± 0.1 | 1.7 ± 0.1 | 1.9 ± 0.1 | 0.55 ± 0.1 |
| Surface 2 | 2.2 ± 0.1 | 2.7 ± 0.4 | 2.5 ± 0.4 | 0.45 ± 0.1 |
| Surface 3 | 2.0 ± 0.1 | 2.8 ± 0.2 | 2.7 ± 0.0 | 0.35 ± 0.2 |
| Surface 4 | 3.0 ± 0.5 | 2.6 ± 0.1 | 2.8 ± 0.3 | 0.40 ± 0.3 |
| Surface 13 | 2.2 ± 0.2 | 3.0 ± 0.1 | 2.7 ± 0.4 | 0.45 ± 0.2 |
| Surface 14 | 1.0 ± 0.1 | 0.8 ± 0.1 | 0.2 ± 0.0 | 0.15 ± 0.1 |
| Surface 15 | 1.9 ± 0.7 | 1.5 ± 1.0 | 0.3* | 0.10* |
| Nunclon | 0.8 ± 0.0 | 1.0 ± 0.0 | 1.9 ± 0.1 | 1.1 ± 0.1 |
| Delta ™ | | | | |
| CellBIND ™ | 1.3 ± 1.0 | 3.0 ± 0.1 | 1.6 ± 2.0 | 0.60 ± 0.0 |
| Falcon ™ | 0.0 ± 0.0 | 0.0 ± 0.1 | 0.6 ± 0.1 | 0.30 ± 0.4 |
| Costar ™ | 0.2 ± 0.0 | 1.0 ± 0.2 | 0.8 ± 0.0 | 0.60 ± 0.1 |
| Primaria ™ | 3.0 ± 0.1 | 0.5 ± 0.1 | 0.5 ± 0.0 | 0.30 ± 0.0 |

Atomic percent of each functional group is given as mean ± standard deviation (n = 2)
*The functional group was identified in only one sample.

TABLE 9
Nitrogen Bonding Environment by N1s Spectra Curve Fitting
Nitrogen Bonding Environment by N1s Spectra Curve Fitting

| | Functional groups and N1s binding energies in eV | | | | |
|---|---|---|---|---|---|
| | —NH₂ | O═C—N—C═O | —NH₃⁺ | —NO₂ | —NO₃ |
| Surface | (398.8 eV) | (400.8 eV) | (401.8 eV) | (406.5 eV) | (407.0 eV) |
| Surface 1-4 | 0.0 | 46.9 ± 2.2 | 43.0 ± 0.4 | 10.2 ± 1.8 | 0.0 |
| and 13* | | | | | |
| Surface 14 | 4.0 ± 1.4 | 75.0 ± 1.4 | 21.0 ± 2.8 | 0.0 | 0.0 |
| Surface 15 | 6.0 ± 1.4 | 76.5 ± 3.5 | 14.0 ± 7.1 | 3.0** | 2.0 ± 0.0 |
| Primaria ™ | 11.0 ± 0.0 | 81.0 ± 0.0 | 4.0 ± 0.0 | 0.0 | 4.0 ± 0.0 |

Atomic percent of each functional group is given as mean ± standard deviation (n = 2)
*N1s spectra were indistinguishable for Surface modified plates 1-4 and 13, and data resulting from curve fit of two representative N1s spectra is given.
**The functional group was identified in only one sample.

TABLE 10
Surface Roughness of the Surface Modified Plates
of the Present Invention as Determined by AFM
Surface Roughness of the Surface Modified Plates
of the Present Invention as Determined by AFM

| | 10 μm × 10 μm scan | | 500 nm × 500 nm scan | |
|---|---|---|---|---|
| Surface | Rₐ (nm) | Rₘₐₓ (nm) | Rₐ (nm) | Rₘₐₓ (nm) |
| Surface | 2.40 | 20.97 | 0.13 | 2.35 |
| modified plate 1 | | | | |
| Surface | 2.27 | 17.38 | 0.42 | 4.40 |
| modified plate 2 | | | | |
| Surface | 2.49 | 22.44 | 0.17 | 2.00 |
| modified plate 3 | | | | |
| Surface | 1.77 | 13.83 | 0.32 | 5.20 |
| modified plate 4 | | | | |
| Surface | 2.14 | 17.99 | 0.18 | 2.30 |
| modified plate 13 | | | | |
| Nunclon Delta ™ | 1.75 | 15.22 | 0.17 | 1.67 |
| Cellbind ™ | 1.63 | 13.04 | 0.17 | 2.10 |

TABLE 11
Summary of the Results of the XPS Analysis of Surface Elemental Composition
and Human Embryonic Stem Cell Attachment and Colony Formation Experiments
on the Surface Modified Plates of the Present Invention

| | | | | | | | hESC Attachment and | |
|---|---|---|---|---|---|---|---|---|
| | | | | hESC Attachment and | | | Colony Formation | |
| | | | | Colony Formation | | | (automated microscopy, | |
| | | Polymer | Surface | (visual inspection) | | | % confluence) | |
| | | Material | Treatment | No RI | Y-27632 | H-1152 | Y-27632 | H-1152 |
| Surface | 1 | PS | CP | − | ++ | ND | 31.4 | ND |
| | 13 | PS | CP | + * | +++ | +++ | 82.7 | ND |
| | 2 | PS | CP | + * | +++ | +++ | 48.6 | ND |
| | 3 | PS | CP | + * | +++ | +++ | 67.5 | ND |
| | 4 | PS | CP | + * | +++ | +++ | 75.3 | ND |
| | 5 | PS | MP | − | + | ND | 2.9 | ND |
| | 6 | PS | MP | − | + | ND | 6.6 | ND |
| | 7 | PS | MP | − | + | ND | 4.3 | ND |
| | 8 | PS | MP | − | + | ND | 5.7 | ND |
| | 9 | PS | MP | − | + | ND | 18.3 | ND |
| | 10 | PS | MP | − | + | ND | 11.5 | ND |
| | 11 | PS | MP | − | + | ND | 10.7 | ND |
| | 12 | PS | MP | − | + | ND | 19.3 | ND |
| | 14 | PS | MP | − | + | ND | ND | ND |
| | 15 | PS | MP | − | + | ND | ND | ND |
| | 18 | COC | None | − | − | − | ND | ND |
| | 19 | COC | CP | − | ++ | ++ | ND | ND |
| | 22 | PS | | − | ND | − | ND | ND |
| | 23 | PS | | − | ND | − | ND | ND |
| | 24 | PS | | − | ND | − | ND | ND |
| | 29 | PS | | − | ND | − | ND | ND |
| | 30 | PS/PC | None | − | ND | − | ND | ND |
| | 33 | PS/PC | CP | − | ND | ++ | ND | ND |
| | 31 | PC | None | − | ND | − | ND | ND |
| | 32 | PC | None | − | ND | − | ND | ND |
| | 34 | PC | CP | − | ND | ++ | ND | ND |
| | CellBIND ™ | PS | | + ** | ++ | ++ | ND | ND |
| | Nunclon Delta ™ | PS | | − | − | − | ND | ND |
| | Falcon ™ | PS | | − | − | − | ND | ND |
| | Costar ™ | PS | | − | − | − | ND | ND |
| | Primaria ™ | PS | | + *** | +++ | +++ | ND | ND |

| | | | | | | | Water contact | |
|---|---|---|---|---|---|---|---|---|
| | | % Nitrogen | | % Oxygen | | Sum of % Nitrogen | angle (degrees) | |
| | | Mean | SD | Mean | SD | and % Oxygen | Mean | SD |
| Surface | 1 | 1.3 | 0.0 | 23.6 | 1.4 | 24.9 | 20.7 | 0.3 |
| | 13 | 1.8 | 0.2 | 26.4 | 2.1 | 28.2 | 18.8 | 0.5 |
| | 2 | 1.7 | 0.1 | 27.9 | 2.0 | 29.6 | 14.3 | 0.4 |
| | 3 | 2.0 | 0.3 | 28.7 | 0.9 | 30.7 | 18.4 | 0.8 |
| | 4 | 2.1 | 0.1 | 28.2 | 0.9 | 30.2 | 17.4 | 2.0 |
| | 5 | 1.3 | 0.2 | 19.6 | 0.2 | 20.9 | 38.7 | 1.1 |
| | 6 | 1.3 | 0.2 | 19.1 | 0.5 | 20.4 | 39.4 | 1.5 |
| | 7 | 1.3 | 0.1 | 20.1 | 1.9 | 21.4 | 38.1 | 1.3 |
| | 8 | 1.5 | 0.1 | 20.0 | 1.0 | 21.5 | 39.0 | 1.5 |
| | 9 | 1.0 | 0.1 | 19.3 | 0.8 | 20.3 | 42.0 | 0.8 |
| | 10 | 0.8 | 0.2 | 19.1 | 0.2 | 19.9 | 41.0 | 1.1 |
| | 11 | 0.9 | 0.1 | 20.3 | 0.1 | 21.2 | 40.0 | 0.7 |
| | 12 | 0.9 | 0.1 | 21.2 | 0.7 | 22.1 | 40.0 | 0.6 |
| | 14 | 1.7 | 0.1 | 15.6 | 0.0 | 17.3 | ND | |
| | 15 | 1.4 | 0.1 | 20.0 | 0.0 | 21.4 | ND | |
| | 18 | 0.0 | 0.0 | 2.3 | 0.1 | 2.3 | ND | |
| | 19 | 1.3 | 0.1 | 25.4 | 1.0 | 26.7 | ND | |
| | 22 | 1.6 | **** | 15.9 | **** | 17.5 | 21.4 | 0.7 |
| | 23 | 1.1 | 0.1 | 17.1 | 1.2 | 18.2 | 30.0 | 2.1 |
| | 24 | 1.3 | 0.4 | 16.7 | 2.7 | 18.0 | 27.7 | 3.3 |
| | 29 | 17.9 | 2.1 | 28.7 | 0.8 | 46.6 | 7.9 | 0.6 |
| | 30 | 0.2 | 0.1 | 3.9 | 0.6 | 4.1 | 70.2 | 0.5 |
| | 33 | 1.0 | 0.1 | 25.0 | 0.2 | 26.0 | 21.8 | 0.5 |
| | 31 | 0.0 | 0.0 | 16.7 | 0.4 | 16.7 | ND | |
| | 32 | 0.0 | 0.0 | 17.0 | 0.4 | 17.0 | ND | |
| | 34 | 1.1 | 0.1 | 23.9 | 0.6 | 25.0 | ND | |
| | CellBIND ™ | 1.0 | 0.1 | 26.8 | 0.1 | 27.8 | 44.3 | 1.3 |
| | Nunclon Delta ™ | 0.6 | 0.1 | 14.7 | 0.0 | 15.3 | 63.1 | 2.0 |
| | Falcon ™ | 0.2 | 0.1 | 4.9 | 0.3 | 5.1 | 75.1 | 2.9 |
| | Costar ™ | 0.3 | 0.1 | 14.1 | 0.4 | 14.4 | 61.4 | 2.4 |
| | Primaria ™ | 10.2 | 0.6 | 12.8 | 0.1 | 23.0 | 39.5 | 2.0 |

“−” means formation of less than 15 colonies per 10 cm²
“+”, “++”, and “+++” means some (15 or more colonies per 10 cm²), more, and most human ES cell attachment and colony formation, respectively
“RI” means Rho kinase inhibitor; “ND” means experiment not done
“PS” means polystyrene; “PC” means polycarbonate; “PS/PC” means blend of polystyrene and polycarbonate; “COC” means cyclic olefin copolymer; “CP” means corona plasma; “MP” means microwave plasma
* Human ES cells attach and grow into colonies that can be passaged about 3 times (then growth rate declines spontaneously)
** Human ES cells attach and grow into colonies that spontaneously differentiate before the first passaging
*** Human ES cells attach and grow into colonies (passaging not tested)
**** Only one sample available for analysis

TABLE 12
Surface Elemental Composition as Determined by XPS
Surface Elemental Composition as Determined by XPS

| | % Carbon | | | % Oxygen | | | % Nitrogen | | |
|---|---|---|---|---|---|---|---|---|---|
| Surface | 1 | 2 | Mean ± SD | 1 | 2 | Mean ± SD | 1 | 2 | Mean ± SD |
| Surface 5 | 79.1 | 79.1 | 79.1 ± 0.0 | 19.5 | 19.8 | 19.6 ± 0.2 | 1.4 | 1.1 | 1.3 ± 0.2 |
| Surface 6 | 79.4 | 79.2 | 79.3 ± 0.1 | 19.5 | 18.8 | 19.1 ± 0.5 | 1.1 | 1.4 | 1.3 ± 0.2 |
| Surface 7 | 76.2 | 80.0 | 78.1 ± 2.7 | 21.5 | 18.8 | 20.1 ± 1.9 | 1.3 | 1.2 | 1.3 ± 0.1 |
| Surface 8 | 77.8 | 78.7 | 78.2 ± 0.6 | 20.8 | 19.3 | 20.0 ± 1.0 | 1.4 | 1.5 | 1.5 ± 0.1 |
| Surface 9 | 79.2 | 80.3 | 79.7 ± 0.8 | 19.9 | 18.7 | 19.3 ± 0.8 | 0.9 | 1.0 | 1.0 ± 0.1 |
| Surface 10 | 80.4 | 79.2 | 79.8 ± 0.8 | 19.0 | 19.3 | 19.1 ± 0.2 | 0.6 | 0.9 | 0.8 ± 0.2 |
| Surface 11 | 78.7 | 78.6 | 78.6 ± 0.1 | 20.4 | 20.3 | 20.3 ± 0.1 | 0.9 | 0.8 | 0.9 ± 0.1 |
| Surface 12 | 77.2 | 78.5 | 77.8 ± 0.9 | 21.8 | 20.7 | 21.2 ± 0.7 | 1.0 | 0.8 | 0.9 ± 0.1 |
| Surface 18* | 97.6 | 97.8 | 97.7 ± 0.1 | 2.4 | 2.2 | 2.3 ± 0.1 | 0.0 | 0.0 | 0.0 ± 0.0 |
| Surface 19 | 74.1 | 72.5 | 73.3 ± 1.1 | 24.7 | 26.2 | 25.4 ± 1.0 | 1.2 | 1.3 | 1.3 ± 0.1 |
| Surface 22 | 82.5 | | | 15.9 | | | 1.6 | | |
| Surface 23** | 82.2 | 79.1 | 80.7 ± 2.2 | 16.2 | 17.9 | 17.1 ± 1.2 | 1.0 | 1.1 | 1.1 ± 0.1 |
| Surface 24** | 84.0 | 79.6 | 81.8 ± 3.1 | 14.8 | 18.6 | 16.7 ± 2.7 | 1.0 | 1.6 | 1.3 ± 0.4 |
| Surface 29 | 52.5 | 54.5 | 53.5 ± 1.4 | 28.1 | 29.2 | 28.7 ± 0.8 | 19.4 | 16.4 | 17.9 ± 2.1 |
| Surface 30* | 96.3 | 95.5 | 95.9 ± 0.6 | 3.5 | 4.4 | 3.9 ± 0.6 | 0.2 | 0.1 | 0.2 ± 0.1 |
| Surface 31*,** | 83.0 | 81.3 | 82.2 ± 1.2 | 16.4 | 17.0 | 16.7 ± 0.4 | 0.0 | 0.0 | 0.0 ± 0.0 |
| Surface 32* | 83.3 | 82.7 | 83.0 ± 0.4 | 16.7 | 17.3 | 17.0 ± 0.4 | 0.0 | 0.0 | 0.0 ± 0.0 |
| Surface 33 | 74.2 | 73.7 | 73.9 ± 0.3 | 24.9 | 25.2 | 25.0 ± 0.2 | 0.9 | 1.0 | 1.0 ± 0.1 |
| Surface 34 | 74.7 | 75.5 | 75.1 ± 0.6 | 24.3 | 23.5 | 23.9 ± 0.6 | 1.1 | 1.0 | 1.1 ± 0.1 |

Measurements on two samples (except Surface 22) and mean ± standard deviation (SD) is given in units of atomic percent
*Not plasma treated.
**Other elements were detected at a concentration of 0.2-2.0%.

TABLE 13
Carbon Bonding Environment by C1s Spectra Curve Fitting
Carbon Bonding Environment by C1s Spectra Curve Fitting

| | Functional groups and C1s binding energies (eV) | | | | | | | | |
|---|---|---|---|---|---|---|---|---|---|
| | | | C—O—C, | | | | | | Energy |
| | C—C | C*—C—O—C—C* | C—NH₃⁺ | C—[O]—C | C═O | C—O—C*═O | CO₃⁻ | O—[C═O]—O | loss peak |
| Surface | (284.6 eV) | (285.2 eV) | (286.1 eV) | (287.0 eV) | (287.9 eV) | (288.9 eV) | (289.8 eV) | (291.0 eV) | (292 eV) |
| Surface 5 | 38.0 | 22.1 | 8.2 | 4.6 | 0.6 | 1.4 | 2.8 | 0.8 | 0.0 |
| Surface 6 | 34.3 | 26.0 | 10.6 | 5.2 | 1.1 | 1.3 | 1.0 | 0.1 | 0.0 |
| Surface 7 | 31.2 | 23.7 | 9.6 | 5.7 | 0.8 | 1.4 | 2.7 | 0.5 | 0.5 |
| Surface 8 | 31.7 | 25.2 | 9.6 | 5.0 | 0.7 | 1.6 | 2.8 | 0.7 | 0.5 |
| Surface 9 | 37.3 | 22.6 | 8.4 | 4.5 | 0.6 | 1.2 | 3.2 | 0.8 | 0.6 |
| Surface 10 | 51.5 | 9.6 | 11.3 | 4.0 | 0.0 | 0.8 | 1.6 | 0.0 | 1.6 |
| Surface 11 | 34.7 | 23.0 | 8.5 | 4.7 | 2.3 | 0.4 | 1.1 | 0.6 | 0.6 |
| Surface 12 | 36.3 | 21.0 | 8.1 | 4.8 | 1.2 | 1.2 | 3.4 | 0.7 | 0.5 |
| Surface 18* | 86.4 ± 1.9 | 0.0 | 9.8 ± 1.4 | 0.0 | 0.0 | 1.5 ± 0.7 | 0.0 | 0.0 | 0.0 |
| Surface 19 | 31.5 ± 1.5 | 18.7 ± 0.3 | 11 ± 0.8 | 10.3 ± 0.1 | 0.0 | 1.8 ± 0.5 | 0.0 | 0.0 | 0.0 |
| Surface 22 | 61.5 | 10.4 | 4.9 | 0.0 | 1.2 | 0.9 | 2.3 | 1.5 | 0.0 |
| Surface 23 | 62.5 ± 4.1 | 9.0 ± 0.7 | 4.2 ± 0.3 | 0.0 | 1.5 ± 0.6 | 0.8 ± 0.1 | 1.9 ± 0.0 | 0.9 ± 0.5 | 0.0 |
| Surface 24 | 66.2 ± 6.1 | 8.8 ± 0.4 | 2.4 ± 0.9 | 0.0 | 1.0 ± 0.6 | 1.2 ± 0.6 | 1.3 ± 0.6 | 1.1 ± 0.1 | 0.0 |
| Surface 29 | 17.8 ± 4.9 | 0.0 | 18.9 ± 1.3 | 5.4 ± 0.9 | 7.4 ± 5.0 | 4.2 ± 0.6 | 0.0 | 0.0 | 0.0 |
| Surface 30* | 84.3 ± 0.5 | 0.0 | 5.7 ± 0.1 | 1.9 ± 0.0 | 0.0 | 0.0 | 0.0 | 0.0 | 3.9 ± 0.1 |
| Surface 31* | 55.3 ± 0.6 | 9.3 ± 0.3 | 9.0 ± 0.1 | 3.5 ± 0.2 | 0.0 | 0.0 | 0.0 | 0.0 | 2.4 ± 0.1 |
| Surface 32* | 51.5 ± 8.5 | 11.2 ± 2.8 | 10.7 ± 3.5 | 3.3 ± 1.1 | 0.0 | 0.0 | 0.0 | 3.3 ± 0.0 | 2.9 ± 0.6 |
| Surface 33 | 27.7 ± 1.7 | 19.9 ± 0.9 | 11.1 ± 0.0 | 11.1 ± 0.0 | 0.0 | 4.1 ± 0.4 | 0.0 | 0.0 | 0.0 |
| Surface 34 | 29.2 ± 0.2 | 21.0 ± 0.1 | 10.9 ± 0.6 | 10.1 ± 0.5 | 0.0 | 3.7 ± 0.1 | 0.0 | 0.0 | 0.0 |

Atomic percent of each functional group is given based on one measurement on Surfaces 5-12 and 22. and as mean ± standard deviation for the other surfaces (n = 2)
*Not plasma treated.

TABLE 14
Nitrogen Bonding Environment by N1s Spectra Curve Fitting
Nitrogen Bonding Environment by N1s Spectra Curve Fitting

| | Functional groups and N1s binding energies in eV | | | | |
|---|---|---|---|---|---|
| | —NH₂ | O═C—N—C═O | —NH₃⁺ | —NO₂ | —NO₃ |
| Surface | (398.8 eV) | (400.8 eV) | (401.8 eV) | (406.5 eV) | (407.0 eV) |
| Surface 5 | 3.0 | 50.0 | 36.0 | 2.0 | 9.0 |
| Surface 6 | 46.0 | 26.0 | 11.0 | 5.0 | 12.0 |
| Surface 7 | 25.0 | 47.0 | 22.0 | 2.0 | 4.0 |
| Surface 8 | 13.0 | 56.0 | 26.0 | 1.0 | 4.0 |
| Surface 9 | 2.0 | 44.0 | 45.0 | 4.0 | 5.0 |
| Surface 10 | 8.0 | 71.0 | 17.0 | 2.0 | 2.0 |
| Surface 11 | 13.0 | 37.0 | 35.0 | 4.0 | 11.0 |
| Surface 12 | 6.0 | 52.0 | 31.0 | 2.0 | 9.0 |
| Surface 18* | ND** | ND | ND | ND | ND |
| Surface 19 | 19.0 ± 8.4 | 51.5 ± 6.3 | 24.0 ± 2.8 | 1.5 ± 0.7 | 4.0 ± 0.0 |
| Surface 22 | 22.0 | 24.0 | 54.0 | 0.0 | 0.0 |
| Surface 23 | 26.0 ± 1.4 | 51.0 ± 4.2 | 23.0 ± 5.7 | 0.0 | 0.0 |
| Surface 24 | 23.0 ± 17.0 | 47.0 ± 5.7 | 30.0 ± 11.3 | 0.0 | 0.0 |
| Surface 29 | 52.0 ± 9.9 | 35.0 ± 15.6 | 6.0 ± 2.8 | 3.5 ± 2.1 | 3.0 ± 1.4 |
| Surface 30* | ND | ND | ND | ND | ND |
| Surface 31* | ND | ND | ND | ND | ND |
| Surface 32* | ND | ND | ND | ND | ND |
| Surface 33 | 7.5 ± 0.7 | 53.5 ± 2.1 | 29.5 ± 0.7 | 5.0 ± 0.0 | 4.5 ± 2.1 |
| Surface 34 | 11.5 ± 2.1 | 55.5 ± 6.4 | 28.0 ± 7.1 | 4.0 ± 1.4 | 1.0 ± 0.0 |

Atomic percent of each functional group is given based on one measurement on Surfaces 5-12 and 22, and as mean ± standard deviation for the other surfaces (n = 2)
*Not plasma treated.
**ND: analyzed, but not detected.

What is claimed is:

1. A method to enhance the attachment of cells to a surface containing from at least about 0.5% N, a sum of O and N of greater than or equal to 17.2% and a contact angle of at least about 13.9 degrees, lacking a feeder cell layer and lacking an adlayer, comprising the steps of:
a. Obtaining a suspension of the cells,
b. Treating the suspension of cells with at least one compound selected from the group consisting of: a compound capable of inhibiting Rho kinase activity, and a compound capable of inhibiting Rho activity, and
c. Adding the suspension of cells to the surface and allowing the cells to attach.

2. The method of claim 1, wherein the sum of O and N is greater than or equal to 19.5%.

3. The method of claim 1, wherein the surface has an adlayer.

4. The method of claim 1, wherein the cells are maintained in culture after the cells attach to the surface.

5. The method of claim 4, wherein at least one compound is removed after the cells attach to the surface.

6. The method of claim 1, wherein the cells are detached from the surface by removing at least one compound.

7. The method of claim 1, wherein the suspension of cells is a suspension of clusters of cells.

8. The method of claim 1, wherein the suspension of cells is a suspension of single cells.

9. The method of claim 1, wherein the surface is part of a vessel or matrix.

10. A method to enhance the attachment of cells to a surface containing from at least about 0.9% N, a sum of O and N of greater than or equal to 22.3% and a contact angle of at least about 13.9 degrees, lacking a feeder cell layer and lacking an adlayer, comprising the steps of:
a. Obtaining a suspension of the cells, and
b. Adding the suspension of cells to the surface and allowing the cells to attach.

11. The method of claim 10, wherein the surface has an adlayer.

12. The method of claim 10, wherein the cells are maintained in culture after the cells attach to the surface.

13. The method of claim 10, wherein the suspension of cells is a suspension of clusters of cells.

14. The method of claim 10, wherein the suspension of cells is a suspension of single cells.

15. The method of claim 10, wherein the surface is part of a vessel or matrix.

16. A surface that is part of a vessel or matrix intended for use in cell culture or analysis, containing from at least about 0.9% N, a sum of O and N of greater than or equal to 22.3% and a contact angle of at least about 13.9 degrees, lacking a feeder cell layer and lacking an adlayer, wherein the surface allows the attachment and cultivation of cells.

17. The method of claim 16, wherein the surface has an adlayer.

18. The surface of claim 16, wherein the cells are maintained in culture after the cells attach to the surface.

19. A composition comprising:
a. A surface that is part of a vessel or matrix intended for use in cell culture or analysis, containing from at least about 0.5% N, a sum of O and N of greater than or equal to 17.2% and a contact angle of at least about 13.9 degrees, lacking a feeder cell layer and lacking an adlayer, and
b. At least one compound selected from the group consisting of: a compound capable of inhibiting Rho kinase activity, and a compound capable of inhibiting Rho activity.

20. The composition of claim 19, wherein the sum of O and N is greater than or equal to 19.5%.

21. The method of claim 19, wherein the surface has an adlayer.
